SEQUENCE LISTING (361 sequences)

SEQ ID NO: 1 - protein, 5 aa, synthetic construct
GYYMX

SEQ ID NO: 2 - protein, 17 aa, synthetic construct
EIXPXXGXAXYNLKFKA

SEQ ID NO: 3 - protein, 6 aa, synthetic construct
LXXLDY

SEQ ID NO: 4 - protein, 16 aa, synthetic construct
RSSQSLVXXXXXTYLH

SEQ ID NO: 5 - protein, 7 aa, synthetic construct
XVSNXXS

SEQ ID NO: 6 - protein, 9 aa, synthetic construct
SQSTXXPXT

SEQ ID NO: 7 - protein, 115 aa, synthetic construct
QVQLVQSGAEVKKPGASVKVSCKASGYSFTGYYMFWVRQAPGQGLEWIGEINPSTGGATYNLKFKARVTLTVDKSTSTAYMELSSLRSEDTAVYYCARLDALDYWGQGTLVTVSS

SEQ ID NO: 8 - protein, 115 aa, synthetic construct
QVQLVQSGAEVKKPGASVKVSCKASGYSFTGYYMNWVRQAPGQGLEWIGEINPSTGGADYNLKFKARVTLTVDKSTSTAYMELSSLRSEDTAVYYCARLSDLDYWGQGTLVTVSS

SEQ ID NO: 9 - protein, 115 aa, synthetic construct
QVQLVQSGAEVKKPGASVKVSCKASGYSFTGYYMGWVRQAPGQGLEWIGEINPSTGGATYNLKFKARVTLTVDKSTSTAYMELSSLRSEDTAVYYCARLSDLDYWGQGTLVTVSS

SEQ ID NO: 10 - protein, 115 aa, synthetic construct
QVQLVQSGAEVKKPGASVKVSCKASGYSFTGYYMSWVRQAPGQGLEWIGEINPSTGGADYNLKFKARVTLTVDKSTSTAYMELSSLRSEDTAVYYCARLADLDYWGQGTLVTVSS

SEQ ID NO: 11 - protein, 115 aa, synthetic construct
QVQLVQSGAEVKKPGASVKVSCKASGYSFTGYYMSWVRQAPGQGLEWIGEINPSTGNADYNLKFKARVTLTVDKSTSTAYMELSSLRSEDTAVYYCARLTDLDYWGQGTLVTVSS

SEQ ID NO: 12 - protein, 115 aa, synthetic construct
QVQLVQSGAEVKKPGASVKVSCKASGYSFTGYYMNWVRQAPGQGLEWIGEINPSTGGATYNLKFKARVTLTVDKSTSTAYMELSSLRSEDTAVYYCARLDALDYWGQGTLVTVSS

SEQ ID NO: 13 - protein, 115 aa, synthetic construct
QVQLVQSGAEVKKPGASVKVSCKASGYSFTGYYMTWVRQAPGQGLEWIGEINPSTGGAHYNLKFKARVTLTVDKSTSTAYMELSSLRSEDTAVYYCARLQDLDYWGQGTLVTVSS

SEQ ID NO: 14 - protein, 115 aa, synthetic construct
QVQLVQSGAEVKKPGASVKVSCKASGYSFTGYYMNWVRQAPGQGLEWIGEINPSTGGAEYNLKFKARVTLTVDKSTSTAYMELSSLRSEDTAVYYCARLDALDYWGQGTLVTVSS

SEQ ID NO: 15 - protein, 115 aa, synthetic construct
QVQLVQSGAEVKKPGASVKVSCKASGYSFTGYYMNWVRQAPGQGLEWIGEINPDTGGATYNLKFKARVTLTVDKSTSTAYMELSSLRSEDTAVYYCARLDALDYWGQGTLVTVSS

SEQ ID NO: 16 - protein, 115 aa, synthetic construct
QVQLVQSGAEVKKPGASVKVSCKASGYSFTGYYMNWVRQAPGQGLEWIGEINPYTGGASYNLKFKARVTLTVDKSTSTAYMELSSLRSEDTAVYYCARLDALDYWGQGTLVTVSS

SEQ ID NO: 17 - protein, 115 aa, synthetic construct
QVQLVQSGAEVKKPGASVKVSCKASGYSFTGYYMNWVRQAPGQGLEWIGEIDPSTGGATYNLKFKARVTLTVDKSTSTAYMELSSLRSEDTAVYYCARLDALDYWGQGTLVTVSS

SEQ ID NO: 18 - protein, 115 aa, synthetic construct
QVQLVQSGAEVKKPGASVKVSCKASGYSFTGYYMSWVRQAPGQGLEWIGEINPSTGEATYNLKFKARVTLTVDKSTSTAYMELSSLRSEDTAVYYCARLTDLDYWGQGTLVTVSS

SEQ ID NO: 19 - protein, 115 aa, synthetic construct
QVQLVQSGAEVKKPGASVKVSCKASGYSFTGYYMSWVRQAPGQGLEWIGEINPGTGGADYNLKFKARVTLTVDKSTSTAYMELSSLRSEDTAVYYCARLSDLDYWGQGTLVTVSS

SEQ ID NO: 20 - protein, 115 aa, synthetic construct
QVQLVQSGAEVKKPGASVKVSCKASGYSFTGYYMYWVRQAPGQGLEWIGEINPSQGGAYYNLKFKARVTLTVDKSTSTAYMELSSLRSEDTAVYYCARLDALDYWGQGTLVTVSS

SEQ ID NO: 21 - protein, 115 aa, synthetic construct
QVQLVQSGAEVKKPGASVKVSCKASGYSFTGYYMSWVRQAPGQGLEWIGEINPGTGGATYNLKFKARVTLTVDKSTSTAYMELSSLRSEDTAVYYCARLSDLDYWGQGTLVTVSS

SEQ ID NO: 22 - protein, 115 aa, synthetic construct
QVQLVQSGAEVKKPGASVKVSCKASGYSFTGYYMSWVRQAPGQGLEWIGEIYPSTGGATYNLKFKARVTLTVDKSTSTAYMELSSLRSEDTAVYYCARLSDLDYWGQGTLVTVSS

SEQ ID NO: 23 - protein, 115 aa, synthetic construct
EVQLQQSGPVLVKPGASVKISCKASGYSFTGYYMFWVKQSPEKSLEWIGEINPSTGGATYNLKFKAKATLTVDKSSNTAYMQLKSLTSEDSAVYYCARLDALDYWGQGTSVTVSS

SEQ ID NO: 24 - protein, 115 aa, synthetic construct
EVQLQQSGPVLVKPGASVKISCKASGYSFTGYYMNWVKQSPEKSLEWIGEINPSTGGATYNLKFKAKATLTVDKSSNTAYMQLKSLTSEDSAVYYCARLDALDYWGQGTSVTVSS

SEQ ID NO: 25 - protein, 115 aa, synthetic construct
QVQLVQSGAEVKKPGASVKVSCKASGYSFTGYYMNWVRQAPGQGLEWIGEINPSTGGADYNLKFKARVTLTVDKSTSTAYMELSSLRSEDTAVYYCARLADLDYWGQGTLVTVSS

SEQ ID NO: 26 - protein, 115 aa, synthetic construct
QVQLVQSGAEVKKPGASVKVSCKASGYSFTGYYMNWVRQAPGQGLEWIGEINPSTGGADYNLKFKARVTLTVDKSTSTAYMELSSLRSEDTAVYYCARLAALDYWGQGTLVTVSS

SEQ ID NO: 27 - protein, 115 aa, synthetic construct
QVQLVQSGAEVKKPGASVKVSCKASGYSFTGYYMNWVRQAPGQGLEWIGEINPSTGGADYNLKFKARVTLTVDKSTSTAYMELSSLRSEDTAVYYCARLDALDYWGQGTLVTVSS

SEQ ID NO: 28 - protein, 115 aa, synthetic construct
QVQLVQSGAEVKKPGASVKVSCKASGYSFTGYYMNWVRQAPGQGLEWIGEINPSTGGADYNLKFKARVTLTVDKSTSTAYMELSSLRSEDTAVYYCARLDDLDYWGQGTLVTVSS

SEQ ID NO: 29 - protein, 115 aa, synthetic construct
QVQLVQSGAEVKKPGASVKVSCKASGYSFTGYYMNWVRQAPGQGLEWIGEINPSTGGATYNLKFKARVTLTVDKSTSTAYMELSSLRSEDTAVYYCARLAALDYWGQGTLVTVSS

SEQ ID NO: 30 - protein, 115 aa, synthetic construct
QVQLVQSGAEVKKPGASVKVSCKASGYSFTGYYMNWVRQAPGQGLEWIGEINPSTGGATYNLKFKARVTLTVDKSTSTAYMELSSLRSEDTAVYYCARLDDLDYWGQGTLVTVSS

SEQ ID NO: 31 - protein, 115 aa, synthetic construct
QVQLVQSGAEVKKPGASVKVSCKASGYSFTGYYMNWVRQAPGQGLEWIGEINPSTGGATYNLKFKARVTLTVDKSTSTAYMELSSLRSEDTAVYYCARLADLDYWGQGTLVTVSS

SEQ ID NO: 32 - protein, 115 aa, synthetic construct
QVQLVQSGAEVKKPGASVKVSCKASGYSFTGYYMNWVRQAPGQGLEWIGEIDPSTGGAEYNLKFKARVTLTVDKSTSTAYMELSSLRSEDTAVYYCARLDALDYWGQGTLVTVSS

SEQ ID NO: 33 - protein, 115 aa, synthetic construct
QVQLVQSGAEVKKPGASVKVSCKASGYSFTGYYMNWVRQAPGQGLEWIGEIDPSTGGADYNLKFKARVTLTVDKSTSTAYMELSSLRSEDTAVYYCARLDALDYWGQGTLVTVSS

SEQ ID NO: 34 - protein, 112 aa, synthetic construct
DVVMTQSPLSLPVTLGQPASISCRSSQSLVHSNGNTYLHWYQQRPGQSPRLLIYRVSNRFSGVPDRFSGSRSGTDFTLKISRVEAEDVGVYFCSQSTHVPFTFGQGTKLEIK

SEQ ID NO: 35 - protein, 112 aa, synthetic construct
DVVMTQSPLSLPVTLGQPASISCRSSQSLVHSNGHTYLHWYQQRPGQSPRLLIYQVSNRFSGVPDRFSGSRSGTDFTLKISRVEAEDVGVYFCSQSTHEPFTFGQGTKLEIK

SEQ ID NO: 36 - protein, 112 aa, synthetic construct
DVVMTQSPLSLPVTLGQPASISCRSSQSLVASNGNTYLHWYQQRPGQSPRLLIYYVSNRFSGVPDRFSGSRSGTDFTLKISRVEAEDVGVYFCSQSTHVPFTFGQGTKLEIK

SEQ ID NO: 37 - protein, 112 aa, synthetic construct
DVVMTQSPLSLPVTLGQPASISCRSSQSLVASNGNTYLHWYQQRPGQSPRLLIYYVSNRFSGVPDRFSGSRSGTDFTLKISRVEAEDVGVYFCSQSTHVPSTFGQGTKLEIK

SEQ ID NO: 38 - protein, 112 aa, synthetic construct
DVVMTQSPLSLPVTLGQPASISCRSSQSLVHSNGNTYLHWYQQRPGQSPRLLIYYVSNRFSGVPDRFSGSRSGTDFTLKISRVEAEDVGVYFCSQSTHSPFTFGQGTKLEIK

SEQ ID NO: 39 - protein, 112 aa, synthetic construct
DVVMTQSPLSLPVTLGQPASISCRSSQSLVHQNGHTYLHWYQQRPGQSPRLLIYQVSNRFSGVPDRFSGSRSGTDFTLKISRVEAEDVGVYFCSQSTHDPFTFGQGTKLEIK

SEQ ID NO: 40 - protein, 112 aa, synthetic construct
DVVMTQSPLSLPVTLGQPASISCRSSQSLVHQNGNTYLHWYQQRPGQSPRLLIYYVSNRHSGVPDRFSGSRSGTDFTLKISRVEAEDVGVYFCSQSTHVPFTFGQGTKLEIK

SEQ ID NO: 41 - protein, 112 aa, synthetic construct
DVVMTQSPLSLPVTLGQPASISCRSSQSLVHSNGHTYLHWYQQRPGQSPRLLIYQVSNRFSGVPDRFSGSRSGTDFTLKISRVEAEDVGVYFCSQSTHSPFTFGQGTKLEIK

SEQ ID NO: 42 - protein, 112 aa, synthetic construct
DVVMTQSPLSLPVTLGQPASISCRSSQSLVHSNGNTYLHWYQQRPGQSPRLLIYQVSNRFSGVPDRFSGSRSGTDFTLKISRVEAEDVGVYFCSQSTADPTTFGQGTKLEIK

SEQ ID NO: 43 - protein, 112 aa, synthetic construct
DVVMTQSPLSLPVTLGQPASISCRSSQSLVESNGNTYLHWYQQRPGQSPRLLIYQVSNRFSGVPDRFSGSRSGTDFTLKISRVEAEDVGVYFCSQSTHEPFTFGQGTKLEIK

SEQ ID NO: 44 - protein, 112 aa, synthetic construct
DVVMTQSPLSLPVTLGQPASISCRSSQSLVHSNGRTYLHWYQQRPGQSPRLLIYQVSNRFSGVPDRFSGSRSGTDFTLKISRVEAEDVGVYFCSQSTHEPYTFGQGTKLEIK

SEQ ID NO: 45 - protein, 112 aa, synthetic construct
DVVMTQSPLSLPVTLGQPASISCRSSQSLVHSNGNTYLHWYQQRPGQSPRLLIYRVSNRFSGVPDRFSGSRSGTDFTLKISRVEAEDVGVYFCSQSTHEPYTFGQGTKLEIK

SEQ ID NO: 46 - protein, 112 aa, synthetic construct
DVVMTQSPLSLPVTLGQPASISCRSSQSLVHSNGNTYLHWYQQRPGQSPRLLIYQVSNRFSGVPDRFSGSRSGTDFTLKISRVEAEDVGVYFCSQSTHEPFTFGQGTKLEIK

SEQ ID NO: 47 - protein, 112 aa, synthetic construct
DVVMTQSPLSLPVTLGQPASISCRSSQSLVDSSGHTYLHWYQQRPGQSPRLLIYRVSNRFSGVPDRFSGSRSGTDFTLKISRVEAEDVGVYFCSQSTHVPFTFGQGTKLEIK

SEQ ID NO: 48 - protein, 112 aa, synthetic construct
DVVMTQSPLSLPVTLGQPASISCRSSQSLVHENGNTYLHWYQQRPGQSPRLLIYQVSNRFSGVPDRFSGSRSGTDFTLKISRVEAEDVGVYFCSQSTEEPFTFGQGTKLEIK

SEQ ID NO: 49 - protein, 112 aa, synthetic construct
DVVMTQSPLSLPVTLGQPASISCRSSQSLVHQRGNTYLHWYQQRPGQSPRLLIYQVSNRFSGVPDRFSGSRSGTDFTLKISRVEAEDVGVYFCSQSTHYPFTFGQGTKLEIK

SEQ ID NO: 50 - protein, 112 aa, synthetic construct
DVVMTQSPLSLPVTLGQPASISCRSSQSLVHSSGHTYLHWYQQRPGQSPRLLIYRVSNRFSGVPDRFSGSRSGTDFTLKISRVEAEDVGVYFCSQSTHVPFTFGQGTKLEIK

SEQ ID NO: 51 - protein, 112 aa, synthetic construct
DVVMTQSPLSLPVTLGQPASISCRSSQSLVHSRGHTYLHWYQQRPGQSPRLLIYRVSNQFSGVPDRFSGSRSGTDFTLKISRVEAEDVGVYFCSQSTHEPFTFGQGTKLEIK

SEQ ID NO: 52 - protein, 112 aa, synthetic construct
DVVMTQSPLSLPVTLGQPASISCRSSQSLVASSGHTYLHWYQQRPGQSPRLLIYRVSNYFSGVPDRFSGSRSGTDFTLKISRVEAEDVGVYFCSQSTHEPFTFGQGTKLEIK

SEQ ID NO: 53 - protein, 112 aa, synthetic construct
DVVMTQTPLSLPVSLGDQASISCRSSQSLVHSNGNTYLHWYLQKPGQSPKLLIYRVSNRFSGVPDRFSGSRSGTDFTLKISRVEAEDLGVYFCSQSTHVPFTFGSGTKLEIK

SEQ ID NO: 54 - protein, 112 aa, synthetic construct
DVVMTQSPLSLPVTLGQPASISCRSSQSLVASRGHTYLHWYQQRPGQSPRLLIYQVSNRFSGVPDRFSGSRSGTDFTLKISRVEAEDVGVYFCSQSTHEPFTFGQGTKLEIK

SEQ ID NO: 55 - protein, 112 aa, synthetic construct
DVVMTQSPLSLPVTLGQPASISCRSSQSLVASRGHTYLHWYQQRPGQSPRLLIYYVSNRFSGVPDRFSGSRSGTDFTLKISRVEAEDVGVYFCSQSTHEPFTFGQGTKLEIK

SEQ ID NO: 56 - protein, 112 aa, synthetic construct
DVVMTQSPLSLPVTLGQPASISCRSSQSLVASNAHTYLHWYQQRPGQSPRLLIYQVSNRFSGVPDRFSGSRSGTDFTLKISRVEAEDVGVYFCSQSTHEPFTFGQGTKLEIK

SEQ ID NO: 57 - protein, 112 aa, synthetic construct
DVVMTQSPLSLPVTLGQPASISCRSSQSLVASNAHTYLHWYQQRPGQSPRLLIYYVSNRFSGVPDRFSGSRSGTDFTLKISRVEAEDVGVYFCSQSTHEPFTFGQGTKLEIK

SEQ ID NO: 58 - protein, 112 aa, synthetic construct
DVVMTQSPLSLPVTLGQPASISCRSSQSLVHSRGHTYLHWYQQRPGQSPRLLIYQVSNRFSGVPDRFSGSRSGTDFTLKISRVEAEDVGVYFCSQSTHEPFTFGQGTKLEIK

SEQ ID NO: 59 - protein, 112 aa, synthetic construct
DVVMTQSPLSLPVTLGQPASISCRSSQSLVHSQGHTYLHWYQQRPGQSPRLLIYQVSNRFSGVPDRFSGSRSGTDFTLKISRVEAEDVGVYFCSQSTHEPFTFGQGTKLEIK

SEQ ID NO: 60 - protein, 112 aa, synthetic construct
DVVMTQSPLSLPVTLGQPASISCRSSQSLVHSHGHTYLHWYQQRPGQSPRLLIYQVSNRFSGVPDRFSGSRSGTDFTLKISRVEAEDVGVYFCSQSTHEPFTFGQGTKLEIK

SEQ ID NO: 61 - protein, 112 aa, synthetic construct
DVVMTQSPLSLPVTLGQPASISCRSSQSLVHSKGHTYLHWYQQRPGQSPRLLIYQVSNRFSGVPDRFSGSRSGTDFTLKISRVEAEDVGVYFCSQSTHEPFTFGQGTKLEIK

SEQ ID NO: 62 - protein, 112 aa, synthetic construct
DVVMTQSPLSLPVTLGQPASISCRSSQSLVASQGHTYLHWYQQRPGQSPRLLIYQVSNRFSGVPDRFSGSRSGTDFTLKISRVEAEDVGVYFCSQSTHEPFTFGQGTKLEIK

SEQ ID NO: 63 - protein, 112 aa, synthetic construct
DVVMTQSPLSLPVTLGQPASISCRSSQSLVASHGHTYLHWYQQRPGQSPRLLIYQVSNRFSGVPDRFSGSRSGTDFTLKISRVEAEDVGVYFCSQSTHEPFTFGQGTKLEIK

SEQ ID NO: 64 - protein, 112 aa, synthetic construct
DVVMTQSPLSLPVTLGQPASISCRSSQSLVASKGHTYLHWYQQRPGQSPRLLIYQVSNRFSGVPDRFSGSRSGTDFTLKISRVEAEDVGVYFCSQSTHEPFTFGQGTKLEIK

SEQ ID NO: 65 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTHEPFTFGQGTKLEIK

SEQ ID NO: 66 - protein, 112 aa, synthetic construct
DVVMTQSPLSLPVTLGQPASISCRSSQSLVHSNAHTYLHWYQQRPGQSPRLLIYYVSNRFSGVPDRFSGSRSGTDFTLKISRVEAEDVGVYFCSQSTHEPFTFGQGTKLEIK

SEQ ID NO: 67 - protein, 112 aa, synthetic construct
DVVMTQSPLSLPVTLGQPASISCRSSQSLVHSQAHTYLHWYQQRPGQSPRLLIYQVSNRFSGVPDRFSGSRSGTDFTLKISRVEAEDVGVYFCSQSTHEPFTFGQGTKLEIK

SEQ ID NO: 68 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTAEPFTFGQGTKLEIK

SEQ ID NO: 69 - protein, 112 aa, synthetic construct
DVVMTQSPLSLPVTLGQPASISCRSSQSLVHSQAHTYLHWYQQRPGQSPRLLIYQVSNRFSGVPDRFSGSRSGTDFTLKISRVEAEDVGVYFCSQSTAEPFTFGQGTKLEIK

SEQ ID NO: 70 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTADPTTFGQGTKLEIK

SEQ ID NO: 71 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTADPFTFGQGTKLEIK

SEQ ID NO: 72 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTHSPFTFGQGTKLEIK

SEQ ID NO: 73 - protein, 112 aa, synthetic construct
DVVMTQSPLSLPVTLGQPASISCRSSQSLVHSNARTYLHWYQQRPGQSPRLLIYQVSNRFSGVPDRFSGSRSGTDFTLKISRVEAEDVGVYFCSQSTHEPYTFGQGTKLEIK

SEQ ID NO: 74 - protein, 112 aa, synthetic construct
DVVMTQSPLSLPVTLGQPASISCRSSQSLVHSNARTYLHWYQQRPGQSPRLLIYQVSNRFSGVPDRFSGSRSGTDFTLKISRVEAEDVGVYFCSQSTHEPFTFGQGTKLEIK

SEQ ID NO: 75 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTHEPYTFGQGTKLEIK

SEQ ID NO: 76 - protein, 112 aa, synthetic construct
DVVMTQSPLSLPVTLGQPASISCRSSQSLVHSQAHTYLHWYQQRPGQSPRLLIYQVSNRFSGVPDRFSGSRSGTDFTLKISRVEAEDVGVYFCSQSTHEPYTFGQGTKLEIK

SEQ ID NO: 77 - protein, 112 aa, synthetic construct
DVVMTQSPLSLPVTLGQPASISCRSSQSLVHSQAHTYLHWYQQRPGQSPRLLIYQVSNRFSGVPDRFSGSRSGTDFTLKISRVEAEDVGVYFCSQSTHSPYTFGQGTKLEIK

SEQ ID NO: 78 - protein, 112 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTHSPYTFGQGTKLEIK

SEQ ID NO: 79 - protein, 5 aa, synthetic construct
GYYMF

SEQ ID NO: 80 - protein, 17 aa, synthetic construct
EINPSTGGATYNLKFKA

SEQ ID NO: 81 - protein, 6 aa, synthetic construct
LDALDY

SEQ ID NO: 82 - protein, 5 aa, synthetic construct
GYYMN

SEQ ID NO: 83 - protein, 17 aa, synthetic construct
EINPSTGGADYNLKFKA

SEQ ID NO: 84 - protein, 6 aa, synthetic construct
LSDLDY

SEQ ID NO: 85 - protein, 5 aa, synthetic construct
GYYMG

SEQ ID NO: 86 - protein, 5 aa, synthetic construct
GYYMS

SEQ ID NO: 87 - protein, 6 aa, synthetic construct
LADLDY

SEQ ID NO: 88 - protein, 6 aa, synthetic construct
LTDLDY

SEQ ID NO: 89 - protein, 5 aa, synthetic construct
GYYMT

SEQ ID NO: 90 - protein, 17 aa, synthetic construct
EINPSTGGAHYNLKFKA

SEQ ID NO: 91 - protein, 6 aa, synthetic construct
LQDLDY

SEQ ID NO: 92 - protein, 17 aa, synthetic construct
EINPSTGGAEYNLKFKA

SEQ ID NO: 93 - protein, 17 aa, synthetic construct
EINPYTGGASYNLKFKA

SEQ ID NO: 94 - protein, 17 aa, synthetic construct
EIDPSTGGATYNLKFKA

SEQ ID NO: 95 - protein, 17 aa, synthetic construct
EIDPSTGGAEYNLKFKA

SEQ ID NO: 96 - protein, 17 aa, synthetic construct
EIDPSTGGADYNLKFKA

SEQ ID NO: 97 - protein, 6 aa, synthetic construct
LDDLDY

SEQ ID NO: 98 - protein, 6 aa, synthetic construct
LAALDY

SEQ ID NO: 99 - protein, 5 aa, synthetic construct
GYYMY

SEQ ID NO: 100 - protein, 17 aa, synthetic construct
EINPSQGGAYYNLKFKA

SEQ ID NO: 101 - protein, 16 aa, synthetic construct
RSSQSLVHSNGNTYLH

SEQ ID NO: 102 - protein, 7 aa, synthetic construct
RVSNRFS

SEQ ID NO: 103 - protein, 9 aa, synthetic construct
SQSTHVPFT

SEQ ID NO: 104 - protein, 16 aa, synthetic construct
RSSQSLVHSNGHTYLH

SEQ ID NO: 105 - protein, 7 aa, synthetic construct
QVSNRFS

SEQ ID NO: 106 - protein, 9 aa, synthetic construct
SQSTHEPFT

SEQ ID NO: 107 - protein, 16 aa, synthetic construct
RSSQSLVASNGNTYLH

SEQ ID NO: 108 - protein, 7 aa, synthetic construct
YVSNRFS

SEQ ID NO: 109 - protein, 9 aa, synthetic construct
SQSTHVPST

SEQ ID NO: 110 - protein, 9 aa, synthetic construct
SQSTHSPFT

SEQ ID NO: 111 - protein, 16 aa, synthetic construct
RSSQSLVHQNGHTYLH

SEQ ID NO: 112 - protein, 9 aa, synthetic construct
SQSTHDPFT

SEQ ID NO: 113 - protein, 16 aa, synthetic construct
RSSQSLVHQNGNTYLH

SEQ ID NO: 114 - protein, 9 aa, synthetic construct
SQSTADPTT

SEQ ID NO: 115 - protein, 16 aa, synthetic construct
RSSQSLVESNGNTYLH

SEQ ID NO: 116 - protein, 16 aa, synthetic construct
RSSQSLVHSNGRTYLH

SEQ ID NO: 117 - protein, 9 aa, synthetic construct
SQSTHEPYT

SEQ ID NO: 118 - protein, 16 aa, synthetic construct
RSSQSLVDSSGHTYLH

SEQ ID NO: 119 - protein, 16 aa, synthetic construct
RSSQSLVHENGNTYLH

SEQ ID NO: 120 - protein, 16 aa, synthetic construct
RSSQSLVHQRGNTYLH

SEQ ID NO: 121 - protein, 16 aa, synthetic construct
RSSQSLVHSSGHTYLH

SEQ ID NO: 122 - protein, 16 aa, synthetic construct
RSSQSLVHSRGHTYLH

SEQ ID NO: 123 - protein, 16 aa, synthetic construct
RSSQSLVASSGHTYLH

SEQ ID NO: 124 - protein, 16 aa, synthetic construct
RSSQSLVASRGHTYLH

SEQ ID NO: 125 - protein, 16 aa, synthetic construct
RSSQSLVASNAHTYLH

SEQ ID NO: 126 - protein, 16 aa, synthetic construct
RSSQSLVHSQGHTYLH

SEQ ID NO: 127 - protein, 16 aa, synthetic construct
RSSQSLVHSHGHTYLH

SEQ ID NO: 128 - protein, 16 aa, synthetic construct
RSSQSLVHSKGHTYLH

SEQ ID NO: 129 - protein, 16 aa, synthetic construct
RSSQSLVASQGHTYLH

SEQ ID NO: 130 - protein, 16 aa, synthetic construct
RSSQSLVASHGHTYLH

SEQ ID NO: 131 - protein, 16 aa, synthetic construct
RSSQSLVASKGHTYLH

SEQ ID NO: 132 - protein, 16 aa, synthetic construct
RSSQSLVHSNAHTYLH

SEQ ID NO: 133 - protein, 16 aa, synthetic construct
RSSQSLVHSQAHTYLH

SEQ ID NO: 134 - protein, 30 aa, synthetic construct
EVQLQQSGPVLVKPGASVKISCKASGYSFT

SEQ ID NO: 135 - protein, 14 aa, synthetic construct
WVKQSPEKSLEWIG

SEQ ID NO: 136 - protein, 32 aa, synthetic construct
KATLTVDKSSNTAYMQLKSLTSEDSAVYYCAR

SEQ ID NO: 137 - protein, 11 aa, synthetic construct
WGQGTSVTVSS

SEQ ID NO: 138 - protein, 32 aa, synthetic construct
RVTLTVDKSISTAYMELSRLRSDDTVVYYCAR

SEQ ID NO: 139 - protein, 30 aa, synthetic construct
QVQLQQWGAGLLKPSETLSLTCAAYGYSFT

SEQ ID NO: 140 - protein, 14 aa, synthetic construct
WIRQPPGKGLEWIG

SEQ ID NO: 141 - protein, 32 aa, synthetic construct
RVTLSVDKSKNQFSLKLSSVTAADTAVYYCAR

SEQ ID NO: 142 - protein, 32 aa, synthetic construct
RVTLSVDKSKNQASLKLSSVTAADTAVYYCAR

SEQ ID NO: 143 - protein, 30 aa, synthetic construct
QVQLQESGPGLVKPSQTLSLTCAAYGYSFT

SEQ ID NO: 144 - protein, 23 aa, synthetic construct
DVVMTQSPLSLPVTLGQPASISC

SEQ ID NO: 145 - protein, 15 aa, synthetic construct
WYQQRPGQSPRLLIY

SEQ ID NO: 146 - protein, 32 aa, synthetic construct
GVPDRFSGSRSGTDFTLKISRVEAEDVGVYFC

SEQ ID NO: 147 - protein, 10 aa, synthetic construct
FGQGTKLEIK

SEQ ID NO: 148 - protein, 23 aa, synthetic construct
DVVMTQTPPSLPVNPGEPASISC

SEQ ID NO: 149 - protein, 15 aa, synthetic construct
WYLQKPGQSPQLLIY

SEQ ID NO: 150 - protein, 32 aa, synthetic construct
GVPDRFSGSRSGTDFTLKISWVEAEDVGVYFC

SEQ ID NO: 151 - protein, 115 aa, synthetic construct
QVQLVQSGAEVKKPGASVKVSCKASGYSFTGYYMFWVRQAPGQGLEWIGEINPSTGGATYNLKFKARVTLTVDKSISTAYMELSRLRSDDTVVYYCARLDALDYWGQGTLVTVSS

SEQ ID NO: 152 - protein, 17 aa, synthetic construct
EINPSTGEATYNLKFKA

SEQ ID NO: 153 - protein, 115 aa, synthetic construct
QVQLQQWGAGLLKPSETLSLTCAAYGYSFTGYYMFWIRQPPGKGLEWIGEINPSTGGATYNLKFKARVTLSVDKSKNQFSLKLSSVTAADTAVYYCARLDALDYWGQGTLVTVSS

SEQ ID NO: 154 - protein, 115 aa, synthetic construct
QVQLQQWGAGLLKPSETLSLTCAAYGYSFTGYYMFWIRQPPGKGLEWIGEINPSTGGATYNLKLKARVTLSVDKSKNQFSLKLSSVTAADTAVYYCARLDALDYWGQGTLVTVSS

SEQ ID NO: 155 - protein, 115 aa, synthetic construct
QVQLQQWGAGLLKPSETLSLTCAAYGYSFTGYYMFWIRQPPGKGLEWIGEINPSTGGATYNLKLKARVTLSVDKSKNQASLKLSSVTAADTAVYYCARLDALDYWGQGTLVTVSS

SEQ ID NO: 156 - protein, 115 aa, synthetic construct
QVQLQESGPGLVKPSQTLSLTCAAYGYSFTGYYMFWIRQPPGKGLEWIGEINPSTGGATYNLKLKARVTLSVDKSKNQFSLKLSSVTAADTAVYYCARLDALDYWGQGTLVTVSS

SEQ ID NO: 157 - protein, 115 aa, synthetic construct
QVQLQESGPGLVKPSQTLSLTCAAYGYSFTGYYMFWIRQPPGKGLEWIGEINPSTGGATYNLKLKARVTLSVDKSKNQASLKLSSVTAADTAVYYCARLDALDYWGQGTLVTVSS

SEQ ID NO: 158 - DNA, 57 bp, synthetic construct
  source 1..57: mol_type = other DNA
atgggctggtcctgcatcatcctgtttctggtggctaccgctaccggcgtgcactct

SEQ ID NO: 159 - protein, 112 aa, synthetic construct
DVVMTQTPPSLPVNPGEPASISCRSSQSLVHSNGNTYLHWYLQKPGQSPQLLIYRVSNRFSGVPDRFSGSRSGTDFTLKISWVEAEDVGVYFCSQSTHVPFTFGQGTKLEIK

SEQ ID NO: 160 - protein, 17 aa, synthetic construct
EINPSTGNADYNLKFKA

SEQ ID NO: 161 - protein, 17 aa, synthetic construct
EINPDTGGATYNLKFKA

SEQ ID NO: 162 - protein, 17 aa, synthetic construct
EINPGTGGADYNLKFKA

SEQ ID NO: 163 - protein, 30 aa, synthetic construct
QVQLVQSGAEVKKPGASVKVSCKASGYSFT

SEQ ID NO: 164 - protein, 14 aa, synthetic construct
WVRQAPGQGLEWIG

SEQ ID NO: 165 - protein, 32 aa, synthetic construct
RVTLTVDKSTSTAYMELSSLRSEDTAVYYCAR

SEQ ID NO: 166 - protein, 11 aa, synthetic construct
WGQGTLVTVSS

SEQ ID NO: 167 - protein, 17 aa, synthetic construct
EINPGTGGATYNLKFKA

SEQ ID NO: 168 - protein, 17 aa, synthetic construct
EIYPSTGGATYNLKFKA

SEQ ID NO: 169 - protein, 7 aa, synthetic construct
YVSNRHS

SEQ ID NO: 170 - protein, 9 aa, synthetic construct
SQSTEEPFT

SEQ ID NO: 171 - protein, 9 aa, synthetic construct
SQSTHYPFT

SEQ ID NO: 172 - protein, 7 aa, synthetic construct
RVSNQFS

SEQ ID NO: 173 - protein, 7 aa, synthetic construct
RVSNYFS

SEQ ID NO: 174 - protein, 9 aa, synthetic construct
SQSTAEPFT

SEQ ID NO: 175 - protein, 9 aa, synthetic construct
SQSTADPFT

SEQ ID NO: 176 - protein, 19 aa, synthetic construct
MGWSCIILFLVATATGVHS

SEQ ID NO: 177 - protein, 9 aa, synthetic construct
SQSTHSPYT

SEQ ID NO: 178 - protein, 23 aa, synthetic construct
DVVMTQTPLSLPVSLGDQASISC

SEQ ID NO: 179 - protein, 15 aa, synthetic construct
WYLQKPGQSPKLLIY

SEQ ID NO: 180 - protein, 32 aa, synthetic construct
GVPDRFSGSRSGTDFTLKISRVEAEDLGVYFC

SEQ ID NO: 181 - protein, 10 aa, synthetic construct
FGSGTKLEIK

SEQ ID NO: 182 - protein, 15 aa, synthetic construct
EGRAAQKRLETLLEE

SEQ ID NO: 183 - protein, 5 aa, synthetic construct
AQKRL

SEQ ID NO: 184 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttactggctactacatgttctgggtccgacaggctcctggacaaggcctggaatggatcggcgagatcaacccttctaccggcggcgccacctacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 185 - DNA, 1395 bp, synthetic construct
  source 1..1395: mol_type = other DNA
atgggctggtcctgcatcatcctgtttctggtggctaccgctaccggcgtgcactctgaggtgcagctacagcagtctgggccagttctggttaagccaggcgcctccgtgaagatctcctgcaaggcctctggctactcttttactggatactacatgttctgggtgaagcagtcccccgagaagtccctggagtggatcggcgagatcaacccttctaccggcggcgccacctacaacctgaagttcaaggccaaggccaccctgaccgtggacaagtcctccaacactgcctacatgcagctgaagtccctgacctccgaggactccgccgtgtactactgcgccagactggatgctctggactattggggccagggcacctctgtgaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 186 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcaacccttctaccggcggcgccacctacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 187 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcactggctactacatgggctgggtccgacaggctcctggacagggcctggaatggatcggcgagatcaacccttctaccggcggcgccacctacaacctgaagttcaaggccagagtgaccctgaccgtggacaagtctaccagcaccgcctacatggagctgtcctccctgagatctgaggacaccgccgtgtactactgcgccagactgtccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 188 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatcaacccttctaccggcggggctgactacaacctgaagttcaaggccagagtgaccctgaccgtggacaagtctaccagcaccgcctacatggagctgtcctccctgagatctgaggacaccgccgtgtactactgcgccagactggccgacctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 189 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatcaacccctctactggcaatgctgactacaacctgaagttcaaggccagagtgaccctgaccgtggacaagtctaccagcaccgcctacatggagctgtcctccctgagatctgaggacaccgccgtgtactactgtgctagactgaccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 190 - DNA, 1395 bp, synthetic construct; identical to SEQ ID NO: 186
  source 1..1395: mol_type = other DNA

SEQ ID NO: 191 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgacctgggtccgacaggctcctggacagggcctggaatggatcggcgagatcaacccttctaccggcggcgcccactacaacctgaagttcaaggccagagtgaccctgaccgtggacaagtctaccagcaccgcctacatggagctgtcctccctgagatctgaggacaccgccgtgtactactgcgccagactgcaggacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 192 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcaacccttctacaggcggagccgagtacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 193 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcaaccctgacacaggcggcgccacctacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 194 - DNA, 1395 bp, synthetic construct; identical to SEQ ID NO: 186
  source 1..1395: mol_type = other DNA

SEQ ID NO: 195 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcaacccctacacaggcggagcctcctacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 196 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcgacccctccaccggcggcgctacctacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 197 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatcaacccctctaccggcgaggccacctacaacctgaagttcaaggccagagtgaccctgaccgtggacaagtctaccagcaccgcctacatggagctgtcctccctgagatctgaggacaccgccgtgtactactgtgctagactgaccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 198 - DNA, 1395 bp, synthetic construct; identical to SEQ ID NO: 186
  source 1..1395: mol_type = other DNA

SEQ ID NO: 199 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatcaaccctggcaccgggggggctgactacaacctgaagttcaaggccagagtgaccctgaccgtggacaagtctaccagcaccgcctacatggagctgtcctccctgagatctgaggacaccgccgtgtactactgcgccagactgtccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 200 - DNA, 1395 bp, synthetic construct; identical to SEQ ID NO: 186
  source 1..1395: mol_type = other DNA

SEQ ID NO: 201 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtactgggtccgacaggctcctggacagggcctggaatggatcggcgagatcaacccttctcagggcggcgcctactacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 202 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcaacccttctaccggcggggctgactacaacctgaagttcaaggccagagtgaccctgaccgtggacaagtctaccagcaccgcctacatggagctgtcctccctgagatctgaggacaccgccgtgtactactgcgccagactgtccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 203 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatcaaccctggcaccggcggggccacctacaacctgaagttcaaggccagagtgaccctgaccgtggacaagtctaccagcaccgcctacatggagctgtcctccctgagatctgaggacaccgccgtgtactactgcgccagactgtccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 204 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactctttcaccggctactacatgtcctgggtccgacaggctcctggacagggcctggaatggatcggcgagatctacccctccaccggcggcgccacctacaacctgaagttcaaggccagagtgaccctgaccgtggacaagtctaccagcaccgcctacatggagctgtcctccctgagatctgaggacaccgccgtgtactactgcgccagactgtccgacctggactactggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 205 - DNA, 1395 bp, synthetic construct; identical to SEQ ID NO: 203
  source 1..1395: mol_type = other DNA

SEQ ID NO: 206 - DNA, 1395 bp, synthetic construct; identical to SEQ ID NO: 204
  source 1..1395: mol_type = other DNA

SEQ ID NO: 207 - DNA, 1395 bp, synthetic construct; identical to SEQ ID NO: 203
  source 1..1395: mol_type = other DNA

SEQ ID NO: 208 - DNA, 1395 bp, synthetic construct; identical to SEQ ID NO: 204
  source 1..1395: mol_type = other DNA

SEQ ID NO: 209 - DNA, 1395 bp, synthetic construct; identical to SEQ ID NO: 186
  source 1..1395: mol_type = other DNA

SEQ ID NO: 210 - DNA, 1395 bp, synthetic construct; identical to SEQ ID NO: 186
  source 1..1395: mol_type = other DNA

SEQ ID NO: 211 - DNA, 1395 bp, synthetic construct; identical to SEQ ID NO: 186
  source 1..1395: mol_type = other DNA

SEQ ID NO: 212 - DNA, 1395 bp, synthetic construct; identical to SEQ ID NO: 186
  source 1..1395: mol_type = other DNA

SEQ ID NO: 213 - DNA, 1395 bp, synthetic construct; identical to SEQ ID NO: 186
  source 1..1395: mol_type = other DNA

SEQ ID NO: 214 - DNA, 1395 bp, synthetic construct; identical to SEQ ID NO: 186
  source 1..1395: mol_type = other DNA

SEQ ID NO: 215 - DNA, 1395 bp, synthetic construct; identical to SEQ ID NO: 186
  source 1..1395: mol_type = other DNA

SEQ ID NO: 216 - DNA, 1395 bp, synthetic construct; identical to SEQ ID NO: 186
  source 1..1395: mol_type = other DNA

SEQ ID NO: 217 - DNA, 1395 bp, synthetic construct; identical to SEQ ID NO: 186
  source 1..1395: mol_type = other DNA

SEQ ID NO: 218 - DNA, 1395 bp, synthetic construct; identical to SEQ ID NO: 186
  source 1..1395: mol_type = other DNA

SEQ ID NO: 219 - DNA, 1395 bp, synthetic construct; identical to SEQ ID NO: 186
  source 1..1395: mol_type = other DNA

SEQ ID NO: 220 - DNA, 1395 bp, synthetic construct; identical to SEQ ID NO: 186
  source 1..1395: mol_type = other DNA

SEQ ID NO: 221 - DNA, 1395 bp, synthetic construct; identical to SEQ ID NO: 186
  source 1..1395: mol_type = other DNA

SEQ ID NO: 222 - DNA, 1395 bp, synthetic construct; identical to SEQ ID NO: 186
  source 1..1395: mol_type = other DNA

SEQ ID NO: 223 - DNA, 1395 bp, synthetic construct; identical to SEQ ID NO: 186
  source 1..1395: mol_type = other DNA

SEQ ID NO: 224 - DNA, 1395 bp, synthetic construct; identical to SEQ ID NO: 186
  source 1..1395: mol_type = other DNA

SEQ ID NO: 225 - DNA, 1395 bp, synthetic construct; identical to SEQ ID NO: 186
  source 1..1395: mol_type = other DNA

SEQ ID NO: 226 - DNA, 1395 bp, synthetic construct; identical to SEQ ID NO: 186
  source 1..1395: mol_type = other DNA

SEQ ID NO: 227 - DNA, 1395 bp, synthetic construct; identical to SEQ ID NO: 186
  source 1..1395: mol_type = other DNA

SEQ ID NO: 228 - DNA, 1395 bp, synthetic construct; identical to SEQ ID NO: 186
  source 1..1395: mol_type = other DNA

SEQ ID NO: 229 - DNA, 1395 bp, synthetic construct; identical to SEQ ID NO: 186
  source 1..1395: mol_type = other DNA

SEQ ID NO: 230 - DNA, 1395 bp, synthetic construct; identical to SEQ ID NO: 186
  source 1..1395: mol_type = other DNA

SEQ ID NO: 231 - DNA, 1395 bp, synthetic construct; identical to SEQ ID NO: 186
  source 1..1395: mol_type = other DNA

SEQ ID NO: 232 - DNA, 1395 bp, synthetic construct; identical to SEQ ID NO: 186
  source 1..1395: mol_type = other DNA

SEQ ID NO: 233 - DNA, 1395 bp, synthetic construct; identical to SEQ ID NO: 186
  source 1..1395: mol_type = other DNA

SEQ ID NO: 234 - DNA, 1395 bp, synthetic construct; identical to SEQ ID NO: 186
  source 1..1395: mol_type = other DNA

SEQ ID NO: 235 - DNA, 1395 bp, synthetic construct; identical to SEQ ID NO: 186
  source 1..1395: mol_type = other DNA

SEQ ID NO: 236 - DNA, 1395 bp, synthetic construct; identical to SEQ ID NO: 186
  source 1..1395: mol_type = other DNA

SEQ ID NO: 237 - DNA, 1395 bp, synthetic construct; identical to SEQ ID NO: 186
  source 1..1395: mol_type = other DNA

SEQ ID NO: 238 - DNA, 1395 bp, synthetic construct; identical to SEQ ID NO: 186
  source 1..1395: mol_type = other DNA

SEQ ID NO: 239 - DNA, 1395 bp, synthetic construct; identical to SEQ ID NO: 186
  source 1..1395: mol_type = other DNA

SEQ ID NO: 240 - DNA, 1395 bp, synthetic construct; identical to SEQ ID NO: 186
  source 1..1395: mol_type = other DNA

SEQ ID NO: 241 - DNA, 1395 bp, synthetic construct; identical to SEQ ID NO: 186
  source 1..1395: mol_type = other DNA

SEQ ID NO: 242 - DNA, 1395 bp, synthetic construct; identical to SEQ ID NO: 186
  source 1..1395: mol_type = other DNA

SEQ ID NO: 243 - DNA, 1395 bp, synthetic construct; identical to SEQ ID NO: 186
  source 1..1395: mol_type = other DNA

SEQ ID NO: 244 - DNA, 1395 bp, synthetic construct; identical to SEQ ID NO: 186
  source 1..1395: mol_type = other DNA

SEQ ID NO: 245 - DNA, 1395 bp, synthetic construct; identical to SEQ ID NO: 186
  source 1..1395: mol_type = other DNA

SEQ ID NO: 246 - DNA, 1395 bp, synthetic construct; identical to SEQ ID NO: 186
  source 1..1395: mol_type = other DNA

SEQ ID NO: 247 - DNA, 1395 bp, synthetic construct; identical to SEQ ID NO: 192
  source 1..1395: mol_type = other DNA

SEQ ID NO: 248 - DNA, 1395 bp, synthetic construct; identical to SEQ ID NO: 192
  source 1..1395: mol_type = other DNA

SEQ ID NO: 249 - DNA, 1395 bp, synthetic construct; identical to SEQ ID NO: 186
  source 1..1395: mol_type = other DNA

SEQ ID NO: 250 - DNA, 1395 bp, synthetic construct; identical to SEQ ID NO: 196
  source 1..1395: mol_type = other DNA

SEQ ID NO: 251 - DNA, 1395 bp, synthetic construct; identical to SEQ ID NO: 196
  source 1..1395: mol_type = other DNA

SEQ ID NO: 252 - DNA, 1395 bp, synthetic construct; identical to SEQ ID NO: 196
  source 1..1395: mol_type = other DNA

SEQ ID NO: 253 - DNA, 1395 bp, synthetic construct; identical to SEQ ID NO: 196
  source 1..1395: mol_type = other DNA

SEQ ID NO: 254 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcgacccctctactggcggcgccgagtacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 255 - DNA, 1395 bp, synthetic construct
  source 1..1395: mol_type = other DNA
atgggctggtcctgcatcatcctgtttctggtggctaccgctaccggcgtgcactctcaggttcagttggttcagtctggcgccgaggtgaagaagcctggcgcctctgtgaaggtgtcctgcaaggcctctggctactcttttaccggctactacatgaactgggtccgacaggctcctggacaaggcctggaatggatcggcgagatcgacccctctactggcggagctgactacaacctgaagttcaaggccagagtgaccctgaccgtggacaagtctaccagcaccgcctacatggagctgtcctccctgagatctgaggacaccgccgtgtactactgcgccagactggatgctctggactattggggccagggcaccctggtcaccgtgtcctctgccagtaccaagggaccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccaagctgacagtggacaagtccagatggcagcagggcaacgtgttctcctgctccgtgatgcacgaggccctgcacaaccactacacccagaagtccctgtctctgagccctggcaagtga

SEQ ID NO: 256 - DNA, 1395 bp, synthetic construct; identical to SEQ ID NO: 254
  source 1..1395: mol_type = other DNA

SEQ ID NO: 257 - DNA, 1395 bp, synthetic construct; identical to SEQ ID NO: 255
  source 1..1395: mol_type = other DNA

SEQ ID NO: 258 - DNA, 1395 bp, synthetic construct; identical to SEQ ID NO: 254
  source 1..1395: mol_type = other DNA

SEQ ID NO: 259 - DNA, 1395 bp, synthetic construct; identical to SEQ ID NO: 255
  source 1..1395: mol_type = other DNA

SEQ ID NO: 260 - DNA, 1395 bp, synthetic construct; identical to SEQ ID NO: 254
  source 1..1395: mol_type = other DNA

SEQ ID NO: 261 - DNA, 1395 bp, synthetic construct; identical to SEQ ID NO: 255
  source 1..1395: mol_type = other DNA

SEQ ID NO: 262 - DNA, 1395 bp, synthetic construct; identical to SEQ ID NO: 196
  source 1..1395: mol_type = other DNA

SEQ ID NO: 263 - DNA, 1395 bp, synthetic construct; identical to SEQ ID NO: 192
  source 1..1395: mol_type = other DNA

SEQ ID NO: 264 - DNA, 1395 bp, synthetic construct; identical to SEQ ID NO: 186
  source 1..1395: mol_type = other DNA

SEQ ID NO: 265 - DNA, 1395 bp, synthetic construct; identical to SEQ ID NO: 186
  source 1..1395: mol_type = other DNA

SEQ ID NO: 266 - DNA, 1395 bp, synthetic construct; identical to SEQ ID NO: 192
  source 1..1395: mol_type = other DNA

SEQ ID NO: 267 - DNA, 1395 bp, synthetic construct; identical to SEQ ID NO: 196
  source 1..1395: mol_type = other DNA

SEQ ID NO: 268 - DNA, 1395 bp, synthetic construct; identical to SEQ ID NO: 196
  source 1..1395: mol_type = other DNA

SEQ ID NO: 269 - DNA, 1395 bp, synthetic construct; identical to SEQ ID NO: 254
  source 1..1395: mol_type = other DNA

SEQ ID NO: 270 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaacggcaacacctacctgcactggtatcagcagagacctggccagtctcctcggctgctgatctaccgggtgtcaaacagattctcaggcgtgcccgacagattctccggctctagatctggcaccgacttcaccctgaagatctccagagtggaagccgaggatgtgggcgtgtacttctgctcccagagcacacatgtgcctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 271 - DNA, 717 bp, synthetic construct
  source 1..717: mol_type = other DNA
atgggctggtcctgcatcatcctgtttctggtggctaccgctaccggcgtgcactctgatgtggtgatgacccagacacctctgagcctgcctgtgtctctgggcgaccaggcctccatctcctgcagatcttctcagtctctggtccactccaacggcaacacctacctgcactggtacctgcagaagcctggccagagccctaagctgctgatctaccgtgtgtcaaaccgtttctcaggcgtgcccgacagattctccggctctagatctggcaccgacttcaccctgaagatctccagagtggaagccgaggacctgggcgtgtacttttgctcccagtctacacatgtgcctttcacctttggctccggcaccaagcttgaaatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 272 - DNA, 717 bp, synthetic construct; identical to SEQ ID NO: 271
  source 1..717: mol_type = other DNA

SEQ ID NO: 273 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctctggtggcatctaatggaaacacctacctgcactggtatcagcagagacctggccagtctcctcggctgctgatctactacgtgtccaacagattttctggcgtgcccgacagattctccggctctagatctggcaccgacttcaccctgaagatctccagagtggaagccgaggatgtgggcgtgtacttctgctcccagagcacacatgtgcctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 274 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctctggtggcatctaatggaaacacctacctgcactggtatcagcagagacctggccagtctcctcggctgctgatctactacgtgtccaacagattttctggcgtgcccgacagattctccggctctagatctggcaccgacttcaccctgaagatctccagagtggaagccgaggatgtgggcgtgtacttttgcagccagagcacccatgtgccttctacctttggccagggcaccaagctggaaatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 275 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaacggcaacacctacctgcactggtatcagcagagacctggccagtctcctcggctgctgatctactacgtgtccaacagattttctggcgtgcccgacagattctccggctctagatctggcaccgacttcaccctgaagatctccagagtggaagccgaggatgtgggcgtgtacttttgcagccagtccacccattctccttttacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 276 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcaccagaatggtcacacttacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacccacgaccccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 277 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtccctggtgcaccagaacggcaacacctacctgcactggtatcagcagagacctggccagtctcctcggctgctgatctactacgtgtccaaccggcactctggcgtgcccgacagattctccggctctagatctggcaccgacttcaccctgaagatctccagagtggaagccgaggatgtgggcgtgtacttctgctcccagagcacacatgtgcctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 278 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactctaacggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttttgcagccagtccacccattctccttttacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 279 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactctaacggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 280 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaacggcaacacctacctgcactggtatcagcagagacctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacagctgaccccaccacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 281 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtggaatctaatggcaacacctacctgcactggtatcagcagagacctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 282 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtgcactctaacggcaggacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacacacgagccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 283 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaacggcaacacctacctgcactggtatcagcagagacctggccagtctcctcggctgctgatctaccgggtgtcaaacagattctcaggcgtgcccgacagattctccggctctagatctggcaccgacttcaccctgaagatctccagagtggaagccgaggatgtgggcgtgtacttctgctcccagtctacacacgagccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 284 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaacggcaacacctacctgcactggtatcagcagagacctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 285 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtggactcctccggccacacctacctgcactggtatcagcagaggcctggccagtctcctcggctgctgatctaccgggtgtcaaacagattctcaggcgtgcccgacagattctccggctctagatctggcaccgacttcaccctgaagatctccagagtggaagccgaggatgtgggcgtgtacttctgctcccagagcacacatgtgcctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 286 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtgcacgagaatggcaacacctacctgcactggtatcagcagagacctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagagcacagaggagcccttcaccttcggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 287 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcaccagcggggcaacacctacctgcactggtatcagcagagacctggccagtctcctcggctgctgatctaccaggtgtccaacagattttctggcgtgcccgacagattctccggctctagatctggcaccgacttcaccctgaagatctccagagtggaagccgaggatgtgggcgtgtacttttgctcccagtctacacactaccctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 288 - DNA, 717 bp, synthetic construct; identical to SEQ ID NO: 279
  source 1..717: mol_type = other DNA

SEQ ID NO: 289 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcctctggccacacctacctgcactggtatcagcagaggcctggccagtctcctcggctgctgatctaccgggtgtcaaacagattctcaggcgtgcccgacagattctccggctctagatctggcaccgacttcaccctgaagatctccagagtggaagccgaggatgtgggcgtgtacttctgctcccagagcacacatgtgcctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 290 - DNA, 717 bp, synthetic construct; identical to SEQ ID NO: 289
  source 1..717: mol_type = other DNA

SEQ ID NO: 291 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctttggttcactccagaggccatacttatctgcactggtatcagcagaggcctggccagtctcctcggctgctgatctaccgggtgtccaaccagttctctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 292 - DNA, 717 bp, synthetic construct; identical to SEQ ID NO: 291
  source 1..717: mol_type = other DNA

SEQ ID NO: 293 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctctggtggcttcttctggccacacctacctgcactggtatcagcagaggcctggccagtctcctcggctgctgatctaccgggtgtccaactacttctccggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 294 - DNA, 717 bp, synthetic construct; identical to SEQ ID NO: 293
  source 1..717: mol_type = other DNA

SEQ ID NO: 295 - DNA, 717 bp, synthetic construct; identical to SEQ ID NO: 270
  source 1..717: mol_type = other DNA

SEQ ID NO: 296 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtggcttctagaggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 297 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtggcttctagaggccacacctacctgcactggtatcagcagaggcctggccagtctcctcggctgctgatctactacgtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 298 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctttggtggcatctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 299 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctttggtggcatctaatgcacacacttacctgcactggtaccagcagaggcctggccagtctcctcggctgctgatctactacgtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 300 - DNA, 717 bp, synthetic construct; identical to SEQ ID NO: 284
  source 1..717: mol_type = other DNA

SEQ ID NO: 301 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctttggttcactccagaggccatacttatctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 302 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcccagggccacacatacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 303 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtgcactcccatggccatacatacctccactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 304 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactccaagggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 305 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctctggtggcctctcagggccacacttacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 306 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcatctcagtctctggtggcttctcatggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 307 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtggcctccaagggccacacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 308 - DNA, 717 bp, synthetic construct; identical to SEQ ID NO: 298
  source 1..717: mol_type = other DNA

SEQ ID NO: 309 - DNA, 717 bp, synthetic construct; identical to SEQ ID NO: 299
  source 1..717: mol_type = other DNA

SEQ ID NO: 310 - DNA, 717 bp, synthetic construct; identical to SEQ ID NO: 298
  source 1..717: mol_type = other DNA

SEQ ID NO: 311 - DNA, 717 bp, synthetic construct; identical to SEQ ID NO: 299
  source 1..717: mol_type = other DNA

SEQ ID NO: 312 - DNA, 717 bp, synthetic construct; identical to SEQ ID NO: 298
  source 1..717: mol_type = other DNA

SEQ ID NO: 313 - DNA, 717 bp, synthetic construct; identical to SEQ ID NO: 299
  source 1..717: mol_type = other DNA

SEQ ID NO: 314 - DNA, 717 bp, synthetic construct; identical to SEQ ID NO: 298
  source 1..717: mol_type = other DNA

SEQ ID NO: 315 - DNA, 717 bp, synthetic construct; identical to SEQ ID NO: 299
  source 1..717: mol_type = other DNA

SEQ ID NO: 316 - DNA, 717 bp, synthetic construct; identical to SEQ ID NO: 298
  source 1..717: mol_type = other DNA

SEQ ID NO: 317 - DNA, 717 bp, synthetic construct; identical to SEQ ID NO: 299
  source 1..717: mol_type = other DNA

SEQ ID NO: 318 - DNA, 717 bp, synthetic construct; identical to SEQ ID NO: 298
  source 1..717: mol_type = other DNA

SEQ ID NO: 319 - DNA, 717 bp, synthetic construct; identical to SEQ ID NO: 299
  source 1..717: mol_type = other DNA

SEQ ID NO: 320 - DNA, 717 bp, synthetic construct; identical to SEQ ID NO: 298
  source 1..717: mol_type = other DNA

SEQ ID NO: 321 - DNA, 717 bp, synthetic construct; identical to SEQ ID NO: 299
  source 1..717: mol_type = other DNA

SEQ ID NO: 322 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 323 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctactacgtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 324 - DNA, 717 bp, synthetic construct; identical to SEQ ID NO: 322
  source 1..717: mol_type = other DNA

SEQ ID NO: 325 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcacaagcacacacctacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 326 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacagcagagcccttcaccttcggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 327 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcacaagcacacacctacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacagcagagcccttcaccttcggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 328 - DNA, 717 bp, synthetic construct; identical to SEQ ID NO: 325
  source 1..717: mol_type = other DNA

SEQ ID NO: 329 - DNA, 717 bp, synthetic construct; identical to SEQ ID NO: 326
  source 1..717: mol_type = other DNA

SEQ ID NO: 330 - DNA, 717 bp, synthetic construct; identical to SEQ ID NO: 327
  source 1..717: mol_type = other DNA

SEQ ID NO: 331 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacagctgaccccaccacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 332 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacagcagaccctttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 333 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttttgcagccagtccacccattctccttttacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 334 - DNA, 717 bp, synthetic construct; identical to SEQ ID NO: 322
  source 1..717: mol_type = other DNA

SEQ ID NO: 335 - DNA, 717 bp, synthetic construct; identical to SEQ ID NO: 333
  source 1..717: mol_type = other DNA

SEQ ID NO: 336 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtgcactcaaacgccaggacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacacacgagccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 337 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtgcactcaaacgccaggacctacctgcactggtatcagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtccacccacgagcccttcacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 338 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacacacgagccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 339 - DNA, 717 bp, synthetic construct; identical to SEQ ID NO: 322
  source 1..717: mol_type = other DNA

SEQ ID NO: 340 - DNA, 717 bp, synthetic construct; identical to SEQ ID NO: 322
  source 1..717: mol_type = other DNA

SEQ ID NO: 341 - DNA, 717 bp, synthetic construct; identical to SEQ ID NO: 322
  source 1..717: mol_type = other DNA

SEQ ID NO: 342 - DNA, 717 bp, synthetic construct; identical to SEQ ID NO: 333
  source 1..717: mol_type = other DNA

SEQ ID NO: 343 - DNA, 717 bp, synthetic construct; identical to SEQ ID NO: 333
  source 1..717: mol_type = other DNA

SEQ ID NO: 344 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcacaagcacacacctacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttctgctcccagtctacacacgagccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 345 - DNA, 717 bp, synthetic construct; identical to SEQ ID NO: 344
  source 1..717: mol_type = other DNA

SEQ ID NO: 346 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggtccactcacaagcacacacctacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttttgcagccagtccacccactctccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 347 - DNA, 717 bp, synthetic construct; identical to SEQ ID NO: 346
  source 1..717: mol_type = other DNA

SEQ ID NO: 348 - DNA, 717 bp, synthetic construct
  source 1..717: mol_type = other DNA
atgggctggtcctgcatcatcctgtttctggtggctaccgctaccggcgtgcactctgatgtggtgatgacccagtctcctctgtctctgcctgtgacattgggccagcctgcctccatctcctgcagatcttctcagtctctggttcactctaatgcacacacttacctgcactggtaccagcagaggcctggccagtctcctcggctgctgatctaccaggtgtccaacagattttctggcgtgcccgacagattctccggctctagatctggcaccgacttcaccctgaagatctccagagtggaagccgaggatgtgggcgtgtacttttgcagccagtccacccactctccttacacctttggccagggcaccaagctggagatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 349 - DNA, 717 bp, synthetic construct; identical to SEQ ID NO: 348
  source 1..717: mol_type = other DNA

SEQ ID NO: 350 - DNA, 717 bp, synthetic construct; identical to SEQ ID NO: 322
  source 1..717: mol_type = other DNA

SEQ ID NO: 351 - DNA, 717 bp, synthetic construct; identical to SEQ ID NO: 322
  source 1..717: mol_type = other DNA

SEQ ID NO: 352 - DNA, 717 bp, synthetic construct; identical to SEQ ID NO: 322
  source 1..717: mol_type = other DNA

SEQ ID NO: 353 - DNA, 717 bp, synthetic construct; identical to SEQ ID NO: 337
  source 1..717: mol_type = other DNA

SEQ ID NO: 354 - DNA, 717 bp, synthetic construct; identical to SEQ ID NO: 338
  source 1..717: mol_type = other DNA

SEQ ID NO: 355 - DNA, 717 bp, synthetic construct; identical to SEQ ID NO: 322
  source 1..717: mol_type = other DNA

SEQ ID NO: 356 - DNA, 660 bp, synthetic construct
  source 1..660: mol_type = other DNA
gacgtcgtgatgacccagtctcctctgtcccttcccgtgaccctggggcagcctgcgtccatctcctgtcggagcagccagagccttgtgcactccaacgcccacacttacctccattggtaccagcagcgcccaggacagtcgccgaggctcctgatctaccaagtgtccaaccggttcagcggagtgcctgatagattcagcggatcgagaagcggaacggactttaccctgaaaatcagccgcgtggaagccgaggacgtgggcgtgtatttctgctcacaatccacccacgagcccttcacattcggacagggcaccaaactcgagatcaagcgcaccgtggcagctccgtcagtgttcatttttccgccctccgacgaacagttgaagtccggtaccgcctcggtggtctgtctgctgaacaatttctacccgcgggaagccaaagtgcagtggaaggtcgataacgcactgcaatcagggaacagccaggaatccgtgactgagcaggactcgaaggattccacttactccctgtcctccaccctgaccctgtcgaaggccgactacgagaagcataaggtctacgcctgcgaagtgacccaccaaggactgtcatcccccgtgactaagtccttcaaccggggcgaatgctga

SEQ ID NO: 357 - DNA, 1338 bp, synthetic construct
  source 1..1338: mol_type = other DNA
caagtgcaactggtgcagagcggagcagaagtgaaaaagcctggagcctccgtaaaagtgtcctgtaaagcgtccggatacagcttcaccggctactacatgaactgggtccgccaagcgccgggccagggcttggaatggatcggagagatcaacccgtccaccggtggcgccgagtataacctcaagttcaaggcccgcgtgaccttgactgtggacaagtccacctccaccgcctacatggaactgagcagcctgagatccgaggatacagccgtgtactattgcgctcggcttgacgctctggactactggggacagggcaccctcgtgactgtgtcgagcgcgtccactaagggtccgtccgtgtttccgctggcccctagctccaagtcaacctcaggaggaaccgccgcactcggttgcctcgtgaaggactacttccccgaacccgtcaccgtgtcctggaatagcggagcgctgacatccggagtgcacacctttcctgcggtgcttcagtcatccggactgtactccctgtcgtctgtggtcactgttccgtcctcctccctggggacccagacttacatttgcaacgtgaaccacaagcccagcaacaccaaggtcgacaagagagtggagccgaagtcctgcgataagacccatacttgccctccatgcccggcccccgaggccgctggaggtccctccgtgttcctcttcccaccaaagcctaaggacaccctgatgatctcgcgcaccccggaagtcacttgtgtggtggtggatgtgtcacatgaggacccagaagtcaagttcaattggtacgtcgacggcgtcgaagtgcacaacgccaagactaagccccgggaggaacagtacaactccacctaccgggtggtgtcggtgctcactgtgctgcaccaggattggctgaatgggaaggagtataagtgcaaagtgtcaaacaaggccttgcccgctcctattgaaaagacgatctcgaaggccaagggccagcctagggagccccaggtctacactctgccgccttcacgagaagagatgaccaagaaccaagtgtcgctgacctgtctcgtgaaagggttttacccctccgacatcgccgtggaatgggaaagcaacggccagccggagaacaactacaagacgacccctcccgtcctggactccgatgggagcttcttcctgtactctaagctgaccgtggacaagagtcggtggcagcaaggaaacgtgttcagctgctcggtcatgcatgaggccctgcacaaccactacactcagaagtcgctcagcctttcgccggggaaatga

SEQ ID NO: 358 - protein, 219 aa, synthetic construct
DVVMTQSPLSLPVTLGQPASISCRSSQSLVHSNAHTYLHWYQQRPGQSPRLLIYQVSNRFSGVPDRFSGSRSGTDFTLKISRVEAEDVGVYFCSQSTHEPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 445 aa, synthetic construct
QVQLVQSGAEVKKPGASVKVSCKASGYSFTGYYMNWVRQAPGQGLEWIGEINPSTGGAEYNLKFKARVTLTVDKSTSTAYMELSSLRSEDTAVYYCARLDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 107 aa, synthetic construct [sequence omitted]

SEQ ID NO: 361 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0226177 A1
(43) Pub. Date: Aug. 6, 2026

(54) ANTIBODIES FOR TREATING NEUROLOGICAL AND NEUROVASCULAR DISORDERS

(71) Applicant: LYS THERAPEUTICS, Caen (FR)

(72) Inventors: Manuel BLANC, Montanay (FR); Francois FOSSIEZ, Lyon (FR); Jordan GUYON, Lyon (FR); Flavie LESEPT, Lyon (FR); Fanny POTZEHA, Lyon (FR); Olivier LEGER, Saint-Sixt (FR)

(21) Appl. No.: 19/290,687

(22) Filed: Aug. 5, 2025

(30) Foreign Application Priority Data
Aug. 5, 2024 (EP) 24192976.9

(51) Int. Cl. C07K 16/28; A61K 9/00; A61K 39/00; A61P 25/28; G01N 33/68

(52) U.S. Cl. CPC C07K 16/286; A61K 9/0019; A61P 25/28; G01N 33/6896; A61K 2039/505; A61K 2039/542; C07K 2317/24; C07K 2317/34; C07K 2317/565; C07K 2317/567; C07K 2317/76; C07K 2317/92; G01N 2333/70571

(57) ABSTRACT

The present disclosure provides humanized anti-GluN1 receptor antibodies and antigen-binding fragments or derivatives thereof, which are effective in inhibiting the deleterious effects of tissue-type plasminogen activator (t-PA) mediated by N-methyl-D-aspartate (NMDA) receptors, as well as pharmaceutical compositions and medical uses thereof, particularly for the treatment of neurological, neurovascular or neurodegenerative disorders, such as stroke, multiple sclerosis, Parkinson's disease, and others.

### CROSS-REFERENCE TO RELATED APPLICATIONS

This application claims the benefit of priority from European application no. 24192976.9 filed Aug. 5, 2024, the contents of which are hereby incorporated by reference in their entirety.

### REFERENCE TO A SEQUENCE LISTING XML

This application contains a Sequence Listing XML, which has been submitted electronically and is hereby incorporated by reference in its entirety. Said Sequence Listing XML is named 107432-022 LSQ.xml, was created on Jul. 24, 2025, and is 495,200 bytes in size.

### FIELD

The disclosure relates generally to the field of antibody-based therapies to prevent NMDA receptor activation by t-PA. More specifically, the disclosure relates to humanized antibodies specific for the GluN1 subunit of the NMDA receptor, pharmaceutical compositions thereof, and methods of use thereof for the prevention or treatment of neurological, neurovascular or neurodegenerative disorders, such as stroke, multiple sclerosis, Parkinson's disease and others.

### BACKGROUND

Tissue-type plasminogen activator (t-PA) has multiple pathophysiological effects in the nervous system, including neuronal toxicity and damage to the blood-brain barrier (BBB), which are mediated by N-methyl-D-aspartate (NMDA) receptors and contribute to a range of neurological and neurodegenerative disorders. t-PA is a serine protease of 69 kDa, and is expressed in endothelial cells, liver cells, and neurons, including in the brain and the spinal cord. It consists of five different functional domains through which it interacts with various substrates, binding proteins and receptors. In the central nervous system (CNS), t-PA can be synthesized and released by virtually all cell types. t-PA displays an array of important functions, which are involved in synaptic plasticity, learning and memory processes, anxiety and neuronal survival or death.

For example, t-PA has been implicated in glutamate excitotoxicity, which is involved in multiple sclerosis (MS) and other demyelinating disorders such as Neuromyelitis optica (NMO, also named Devic's disease) or idiopathic transverse myelitis. t-PA may promote demyelination through its effects on plasmin production; promote excitotoxic neuronal cell death; and contribute to glutamate-induced oligodendrocyte injury and to axonal damage in MS. Several groups have suggested a relationship between t-PA and glutamatergic transmission (for review, see Yepes, M. et al., 2009, Trends Neurosci., 32(1):48-55). t-PA is also overexpressed in stroke patients and likely involved in the pathophysiology of many other diseases. Of note, the various functions of t-PA in the body forbid an approach by which t-PA would be totally neutralised. This is supported by reports that t-PA-KO mice, after a delayed onset of symptomatic disease, exhibit increased severity and delayed recovery in the Experimental Autoimmune Encephalomyelitis (EAE) model of MS (Lu, L. et al., 2002, J Neurosci., 22(24):10781-9).

NMDA receptors (NMDARs) belong to the glutamate receptor family, the primary excitatory neurotransmitter receptors of the central nervous system. They are highly expressed in neurons as well as in a wide range of non-neuronal cells, including central and peripheral glial cells, and endothelial cells of the vascular system. Most of our understanding about NMDAR structure and function comes from studies conducted on neuronal NMDARs.

NMDARs are tetrameric assemblies composed of dimers of obligatory GluN1 and GluN2 (GluN2A-D) subunits. GluN3 (A-B) dimers can replace GluN2 dimers. The extracellular region of all NMDARs consists of eight clamshell-like domains arranged in two layers: a distal N-terminal domain (ATD or NTD) layer and a layer of four agonist-binding domains (ABDs) directly connected to the transmembrane domain (TMD). Typically, glutamate binds to the GluN2 subunit and glycine, the co-agonist, binds to the GluN1 and/or GluN3 subunit.

On neurons, subunit composition and synaptic versus extrasynaptic localization critically influence NMDAR functions, including Ca²⁺ permeability and activation. Moreover, NMDAR and its lateral diffusion at the cell membrane can be modulated by extracellular factors such as matrix metalloproteases or co-agonists.

Over the past three decades, non-neuronal NMDARs have been identified on endothelial cells (Hogan-Cann and Anderson, 2016). Within the vascular compartment, NMDARs are expressed on the luminal surface of endothelial cells and in close proximity to tight-junction proteins in both rodent and human tissues (Macrez, R., Stys, P. K. et al., 2016, Lancet Neurol., 15(10):1089-102; Reijerkerk, A. et al., 2010, J Neurochem., 113(2):447-53). Endothelial NMDARs are likely important regulators of blood-brain and blood-spinal cord barrier maintenance and permeability, oxidative stress, neurovascular inflammatory processes, immune cell transmigration, mitochondrial function, nitric oxide (NO) generation, and are integral components of the neurovascular unit (Seillier, C., Lesept, F. et al., 2022, Int J Mol Sci., 23(18):10336-74). Several studies suggest that NMDARs expressed by cerebrovascular endothelium could also directly mediate vasodilation, regulate cerebral blood flow, and modulate hemodynamic responses to stimuli (Jeanneret, V. and Yepes, M., 2017, Neural Regen Res., 12(3):362-5; Lu, L. et al., 2019, J Cereb Blood Flow Metab., 39(3):481-96).

Lesept et al. (Cell Death and Disease (2016), 7, e2466; doi:10.1038/cddis.2016.279) showed that extracellular neuronal t-PA leads to a selective increase of the surface dynamics and subsequent diffusion of extrasynaptic NMDARs, underlying the ability of t-PA to promote NMDAR-mediated calcium influx. Lesept, F. et al., 2016, Cell Death Dis. 2016, 7(11):e2466 developed a murine monoclonal antibody (named “glunomab”) capable of specifically blocking the interaction of t-PA with the amino terminal domain (ATD) of the GluN1 subunit of NMDAR. Using this antibody, they demonstrated that t-PA binds to an epitope in the ATD of the GluN1 subunit which includes a critical lysine in position 178. (¹⁷⁶AQKRL¹⁸⁰). When applied to mouse neurons, glunomab leads to a selective reduction of the t-PA-mediated surface dynamics of extrasynaptic NMDARs and subsequent signaling and neurotoxicity, both in vitro and in vivo. These results showed that t-PA is a ligand of the ATD of the obligatory GluN1 subunit of NMDAR and acts as a modulator of its dynamic distribution at the cell surface and subsequent signaling.

Blocking the potentiating effect of t-PA on NMDARs could therefore be useful therapeutically to provide neuroprotection and to treat a wide variety of neurological, neurovascular and/or neurodegenerative disorders.

International Application No. PCT/EP2014/060486 (WO/2014/187879) provides anti-NMDAR antibodies or fragments or derivatives thereof which are effective in inhibiting the deleterious effects of t-PA mediated by NMDARs, and medical uses thereof for the treatment of neurological or neurodegenerative disorders.

There remains a need for anti-GluN1 antibodies suitable for clinical use in humans and presenting improved features compared to glunomab, such as a higher affinity for the target.

### SUMMARY

The present disclosure provides improved antibody-based therapies that inhibit NMDA receptor (NMDAR) activation by t-PA. In particular, the present disclosure provides improved antibody-based therapies that inhibit NMDAR activation by t-PA; for use as a medicament.

There are provided anti-GluN1 antibodies or antigen-binding fragments or derivatives thereof suitable for clinical use in humans, as well as compositions comprising the same and methods of using the same for treatment and/or prevention of neurological, neurovascular and/or neurodegenerative disorders.

The antibodies and antigen-binding fragments or derivatives thereof bind specifically to the GluN1 subunit of the NMDAR and inhibit the interaction between the NMDAR and t-PA, thereby inhibiting deleterious effects of t-PA mediated by the NMDAR. By selectively blocking only certain functions of the NMDAR (and of t-PA consequently), the antibodies and antigen-binding fragments or derivatives can avoid causing other undesired or harmful effects resulting from blocking all functions of the NMDAR and/or t-PA.

There are provided herein methods for treatment and prevention of neurological, neurovascular and/or neurodegenerative disorders using the described antibodies and antigen-binding fragments or derivatives thereof, as well as pharmaceutical compositions comprising the same. In certain embodiments, antibodies and antigen-binding fragments or derivatives thereof may exhibit one or more of the following advantages, compared to previously known anti-GluN1 antibodies: increased therapeutic efficacy for treatment and/or prevention of neurological, neurovascular and neurodegenerative disorders; increased therapeutic index for treatment and/or prevention of neurological, neurovascular and neurodegenerative disorders; increased binding affinity and/or specificity to the antigen or epitope; increased ease of manufacturing, e.g., increased yield in CMC manufacturing; increased stability; and/or reduced aggregation.

In a first broad aspect, there are provided humanized anti-GluN1 antibodies or antigen-binding fragments or derivatives thereof which are specific for the GluN1 subunit of the NMDAR. Without wishing to be limited by theory, anti-GluN1 antibodies and antigen-binding fragments or derivatives thereof of the disclosure inhibit interaction between the NMDAR and t-PA and/or inhibit NMDAR activation by t-PA.

In some embodiments, the humanized anti-GluN1 antibodies or antigen-binding fragments or derivatives thereof are capable of specifically binding to an epitope represented by the amino acid sequence EGRAAQKRLETLLEE (SEQ ID NO: 182). In other embodiments, the humanized anti-GluN1 antibodies or antigen-binding fragments or derivatives thereof are capable of specifically binding to an epitope represented by the amino acid sequence AQKRL (SEQ ID NO: 183). In some embodiments, the humanized anti-GluN1 antibodies or antigen-binding fragments or derivatives thereof are capable of competing with glunomab for binding to its target or epitope.

In some embodiments, the humanized anti-GluN1 antibodies or antigen-binding fragments or derivatives thereof comprise:
(i) a heavy chain framework region comprising: a heavy chain FR1 comprising or consisting of the amino acid sequence shown in SEQ ID NO: 163, a heavy chain FR2 comprising or consisting of the amino acid sequence shown in SEQ ID NO: 164, a heavy chain FR3 comprising or consisting of the amino acid sequence shown in SEQ ID NO: 165, a heavy chain FR4 comprising or consisting of the amino acid sequence shown in SEQ ID NO: 166; or one or more heavy chain FR(s) having an amino acid sequence selected from the group consisting of SEQ ID NOs: 163-166 which is substituted at one or two amino acid positions or which contains a substitution of one or more amino acids or which is at least 80% identical to the amino acid sequence set forth in any one of SEQ ID NOs: 163-166, and which is functionally equivalent to the corresponding unaltered heavy chain FR(s);
   and
(ii) a light chain framework region comprising: a light chain FR1 comprising or consisting of the amino acid sequence shown in SEQ ID NO: 144, a light chain FR2 comprising or consisting of the amino acid sequence shown in SEQ ID NO: 145, a light chain FR3 comprising or consisting of the amino acid sequence shown in SEQ ID NO: 146, a light chain FR4 comprising or consisting of the amino acid sequence shown in SEQ ID NO: 147; or one or more light chain FR(s) having an amino acid sequence selected from the group consisting of SEQ ID NOs: 144-147 which is substituted at one or two amino acid positions or which contains a substitution of one or more amino acids or which is at least 80% identical to the amino acid sequence set forth in any one of SEQ ID NOs: 144-147, and which is functionally equivalent to the corresponding unaltered light chain FR(s).

In some embodiments, the humanized anti-GluN1 antibodies or antigen-binding fragments or derivatives thereof comprise:
(i) a heavy chain framework region comprising: a heavy chain FR1 comprising or consisting of the amino acid sequence shown in SEQ ID NO: 163, a heavy chain FR2 comprising or consisting of the amino acid sequence shown in SEQ ID NO: 164, a heavy chain FR3 comprising or consisting of the amino acid sequence shown in SEQ ID NO: 165, a heavy chain FR4 comprising or consisting of the amino acid sequence shown in SEQ ID NO: 166;
   and
(ii) a light chain framework region comprising: a light chain FR1 comprising or consisting of the amino acid sequence shown in SEQ ID NO: 144, a light chain FR2 comprising or consisting of the amino acid sequence shown in SEQ ID NO: 145, a light chain FR3 comprising or consisting of the amino acid sequence shown in SEQ ID NO: 146, a light chain FR4 comprising or consisting of the amino acid sequence shown in SEQ ID NO: 147.

In some embodiments, the humanized anti-GluN1 antibodies or antigen-binding fragments or derivatives thereof comprise: (a) one or more complementarity determining regions (CDRs) comprising or consisting of the amino acid sequence set forth in SEQ ID NO: 79 (CDR-H1), SEQ ID NO: 80 (CDR-H2), SEQ ID NO: 81 (CDR-H3), SEQ ID NO: 101 (CDR-L1), SEQ ID NO: 102 (CDR-L2), and/or SEQ ID NO: 103 (CDR-L3); or, one or more CDRs having an amino acid sequence selected from the group consisting of SEQ ID NOs: 79-81 and 101-103 which are substituted at one or two amino acid positions or which contain a substitution of one or more amino acids, and which are functionally equivalent to the corresponding unaltered CDRs; (b) one or more heavy chain framework regions (FRs) comprising or consisting of the amino acid sequence set forth in SEQ ID NO: 163 (FR1), SEQ ID NO: 164 (FR2), SEQ ID NO: 165 (FR3), and/or SEQ ID NO: 166 (FR4); or a heavy chain FR comprising or consisting of the amino acid sequences set forth in SEQ ID NOs: 163, 164, 165, and/or 166, or a heavy chain FR having an amino acid sequence which is at least 80% identical to the amino acid sequence set forth in any one of SEQ ID NOs: 163-166; and (c) one or more light chain framework regions (FRs) comprising or consisting of the amino acid sequence set forth in SEQ ID NO: 144 (FR1), SEQ ID NO: 145 (FR2), SEQ ID NO: 146 (FR3) and/or SEQ ID NO:147; or a light chain FR comprising or consisting of the amino acid sequences set forth in SEQ ID NO: 144, 145, 146 and/or 147, or a light chain FR having an amino acid sequence which is at least 80% identical to the amino acid sequence set forth in any one of SEQ ID NOs: 144-147.

In some embodiments, the humanized anti-GluN1 antibody, antigen-binding fragment or derivative thereof is a monoclonal antibody.

In some embodiments, the humanized anti-GluN1 antibody, antigen-binding fragment or derivative thereof is capable of binding to at least a portion of a region of an NMDAR that interacts with t-PA, wherein said binding inhibits the interaction between the NMDAR and t-PA, such that at least one function of the NMDAR is selectively inhibited.

In some embodiments, a humanized anti-GluN1 antibody, antigen-binding fragment or derivative thereof comprises a heavy chain variable region (VH) polypeptide comprising or consisting of any one of the amino acid sequences set forth in SEQ ID NOs: 7-22 or 25-33, or an amino acid sequence having at least or about 75%, at least or about 80%, at least or about 85%, at least or about 90%, at least or about 95%, at least or about 97%, at least or about 98%, or at least or about 99% identity thereto.

In some embodiments, an antibody or antigen-binding fragment or derivative thereof comprises a light chain variable region (VL) polypeptide comprising or consisting of any one of the amino acid sequences set forth in SEQ ID NOs: 34-52 or 54-78, or an amino acid sequence having at least or about 75%, at least or about 80%, at least or about 85%, at least or about 90%, at least or about 95%, at least or about 97%, at least or about 98%, or at least or about 99% identity thereto.

In some embodiments, an antibody or antigen-binding fragment or derivative thereof comprises one or more amino acid sequence(s) listed in Table 1, or one or more amino acid sequence(s) having at least or about 75%, at least or about 80%, at least or about 85%, at least or about 90%, at least or about 95%, at least or about 97%, at least or about 98%, or at least or about 99% identity thereto, and one or more amino acid sequence(s) listed in Table 2, or one or more amino acid sequence(s) having at least or about 75%, at least or about 80%, at least or about 85%, at least or about 90%, at least or about 95%, at least or about 97%, at least or about 98%, or at least or about 99% identity thereto.

In some embodiments, the antibody or antigen-binding fragment or derivative thereof of the present disclosure specifically binds to the GluN1 subunit of the NMDAR. In some such embodiments, the antibody or antigen-binding fragment or derivative thereof inhibits or blocks the interaction between the NMDAR and t-PA. In some such embodiments, at least one function of the NMDAR activated by t-PA is selectively inhibited by the antibody or antigen-binding fragment or derivative thereof. In some such embodiments, the antibody or antigen-binding fragment or derivative thereof inhibits or blocks t-PA induced neurotoxicity or neuronal cell death which is mediated by the NMDAR.

In some embodiments, the antibody or antigen-binding fragment or derivative thereof of the present disclosure comprises: a) a combination of a heavy chain CDR1, CDR2, and CDR3 as set forth in Table 3, or a variant sequence thereof which differs by only one or two amino acids, or which has at least or about 85% sequence identity thereto; or b) a combination of a light chain CDR1, CDR2, and CDR3 as set forth in Table 4, or a variant sequence thereof which differs by only one or two amino acids, or which has at least or about 85% sequence identity thereto.

In some embodiments, the antibody or antigen-binding fragment or derivative thereof of the present disclosure comprises: a) a combination of a heavy chain FR1, FR2, FR3, and FR4 as set forth in Table 5, or a variant sequence thereof which differs by only one or two amino acids, or which has at least or about 85% sequence identity thereto; or b) a combination of a light chain FR1, FR2, FR3, and FR4 as set forth in Table 6, or a variant sequence thereof which differs by only one or two amino acids, or which has at least or about 85% sequence identity thereto.

In some embodiments, the antibody or antigen-binding fragment or derivative thereof of the present disclosure comprises: (1) a combination of a heavy chain CDR1, CDR2, and CDR3 as set forth in Table 3, or a variant sequence thereof which differs by only one or two amino acids, or which has at least or about 85% sequence identity thereto; and/or a combination of a light chain CDR1, CDR2, and CDR3 as set forth in Table 4, or a variant sequence thereof which differs by only one or two amino acids, or which has at least or about 85% sequence identity thereto; and (2) a combination of a heavy chain FR1, FR2, FR3, and FR4 as set forth in Table 5, or a variant sequence thereof which differs by only one or two amino acids, or which has at least or about 85% sequence identity thereto; and/or a combination of a light chain FR1, FR2, FR3, and FR4 as set forth in Table 6, or a variant sequence thereof which differs by only one or two amino acids, or which has at least or about 85% sequence identity thereto.

In some embodiments, the antibody or antigen-binding fragment or derivative thereof of the present disclosure comprises a heavy chain CDR1 (H-CDR1) which comprises or consists of the following sequence:
N term-GYYMX₁-C term (SEQ ID NO: 1)
   wherein X₁ is F, N, S, G, T or Y. In some embodiments, X₁ is N, S, G, T or Y. In some embodiments, X₁ is N, S, G or T. In some embodiments, X₁ is F. In some embodiments, X₁ is N. In some embodiments, X₁ is S. In some embodiments, X₁ is G. In some embodiments, X₁ is T. In some embodiments, X₁ is Y.

In some embodiments, the antibody or antigen-binding fragment or derivative thereof of the present disclosure comprises a heavy chain CDR2 (H-CDR2) which comprises or consists of the following sequence:

(SEQ ID NO: 2)
N term-EIX₂PX₃X₁₉GX₁₈AX₄YNLKFKA-C term

wherein X₂ is N, D or Y; X₃ is S, G, D or Y; X₁₉ is T or Q; X₁₈ is G, N or E; and X₄ is T, D, H, E, S or Y. In some embodiments, X₂ is N, X₃ is S, and X₄ is D, H, E, S or Y. In some embodiments, X₂ is N, X₃ is S, and X₄ is D. In some embodiments, X₂ is N, X₃ is S, and X₄ is H. In some embodiments, X₂ is N, X₃ is S, and X₄ is E. In some embodiments, X₂ is N, X₃ is S, and X₄ is S. In some embodiments, X₂ is N, X₃ is S, and X₄ is Y. In some embodiments, X₁₉ is T. In some embodiments, X₁₉ is Q. In some embodiments, X₁₈ is G. In some embodiments, X₁₈ is N. In some embodiments, X₁₈ is E. In some embodiments, X₂ is N, X₃ is S, X₄ is D, X₁₉ is T, and X₁₈ is G, N or E. In some embodiments, X₂ is N, X₃ is S, X₄ is D, X₁₉ is Q, and X₁₈ is G, N or E. In some embodiments, X₂ is N, X₃ is S, X₄ is H, X₁₉ is T, and X₁₈ is G, N or E. In some embodiments, X₂ is N, X₃ is S, X₄ is H, X₁₉ is Q, and X₁₈ is G, N or E. In some embodiments, X₂ is N, X₃ is S, X₄ is E, X₁₉ is T, and X₁₈ is G, N or E. In some embodiments, X₂ is N, X₃ is S, X₄ is E, X₁₉ is Q, and X₁₈ is G, N or E. In some embodiments, X₂ is N, X₃ is S, X₄ is S, X₁₉ is T, and X₁₈ is G, N or E. In some embodiments, X₂ is N, X₃ is S, X₄ is S, X₁₉ is Q, and X₁₈ is G, N or E. In some embodiments, X₂ is N, X₃ is S, X₄ is Y, X₁₉ is T and X₁₈ is G, N or E. In some embodiments, X₂ is N, X₃ is S, X₄ is Y, X₁₉ is Q and X₁₈ is G, N or E. In some embodiments, X₂ is N, X₃ is S, X₄ is D, X₁₉ is T or Q, and X₁₈ is G. In some embodiments, X₂ is N, X₃ is S, X₄ is D, X₁₉ is T or Q, and X₁₈ is N. In some embodiments, X₂ is N, X₃ is S, X₄ is D, X₁₉ is T or Q, and X₁₈ is E. In some embodiments, X₂ is N, X₃ is S, X₄ is H, X₁₉ is T or Q, and X₁₈ is G. In some embodiments, X₂ is N, X₃ is S, X₄ is H, X₁₉ is T or Q, and X₁₈ is N. In some embodiments, X₂ is N, X₃ is S, X₄ is H, X₁₉ is T or Q, and X₁₈ is E. In some embodiments, X₂ is N, X₃ is S, X₄ is E, X₁₉ is T or Q, and X₁₈ is G. In some embodiments, X₂ is N, X₃ is S, X₄ is E, X₁₉ is T or Q, and X₁₈ is N. In some embodiments, X₂ is N, X₃ is S, X₄ is E, X₁₉ is T or Q, and X₁₈ is E. In some embodiments, X₂ is N, X₃ is S, X₄ is S, X₁₉ is T or Q, and X₁₈ is G. In some embodiments, X₂ is N, X₃ is S, X₄ is S, X₁₉ is T or Q, and X₁₈ is N. In some embodiments, X₂ is N, X₃ is S, X₄ is S, X₁₉ is T or Q, and X₁₈ is E. In some embodiments, X₂ is N, X₃ is S, X₄ is Y, X₁₉ is T or Q, and X₁₈ is G. In some embodiments, X₂ is N, X₃ is S, X₄ is Y, X₁₉ is T or Q, and X₁₈ is N. In some embodiments, X₂ is N, X₃ is S, X₄ is Y, X₁₉ is T or Q, and X₁₈ is E.

In some embodiments, the antibody or antigen-binding fragment or derivative thereof of the present disclosure comprises a heavy chain CDR3 (H-CDR3) which comprises or consists of the following sequence:

(SEQ ID NO: 3)
N term-LX₅X₆LDY-C term

wherein X₅ is D, S, A, T, or Q; and X₆ is A or D. In some embodiments X₅ is D and X₆ is A. In some embodiments, X₅ is D. In some embodiments, X₅ is S. In some embodiments, X₅ is A. In some embodiments, X₅ is T. In some embodiments, X₅ is Q. In some embodiments, X₆ is A. In some embodiments, X₆ is D.

In some embodiments, the antibody or antigen-binding fragment or derivative thereof of the present disclosure comprises a light chain CDR1 (L-CDR1) which comprises or consists of the following sequence:

(SEQ ID NO: 4)
N term-RSSQSLVX₇X₁₆X₈X₉X₁₀TYLH-C term

wherein X₇ is H, A, E or D; X₁₆ is S, E or Q; X₈ is N, R, S, Q, H or K; X₉ is G or A; and X₁₀ is N, H or R. In some embodiments X₇ is H, X₈ is N, X₉ is A and X₁₀ is H. In some embodiments X₁₆ is S. In some embodiments X₁₆ is E. In some embodiments X₁₆ is Q. In some embodiments X₇ is H, X₈ is N, X₉ is A, X₁₀ is H, and X₁₆ is S. In some embodiments X₇ is H, X₈ is N, X₉ is A, X₁₀ is H, and X₁₆ is E. In some embodiments X₇ is H, X₈ is N, X₉ is A, X₁₀ is H, and X₁₆ is Q.

In some embodiments, the antibody or antigen-binding fragment or derivative thereof of the present disclosure comprises a light chain CDR2 (L-CDR2) which comprises or consists of the following sequence:

(SEQ ID NO: 5)
N term-X₁₁VSNX₁₂X₁₇S- term

wherein X₁₁ is R, Q or Y; X₁₂ is R, Q or Y; and X₁₇ is F or H. In some embodiments X₁₁ is Q and X₁₂ is R. In some embodiments, X₁₇ is F. In some embodiments, X₁₇ is H. In some embodiments, X₁₁ is Q, X₁₂ is R and X₁₇ is F. In some embodiments, X₁₁ is Q, X₁₂ is R and X₁₇ is H.

In some embodiments, the antibody or antigen-binding fragment or derivatives thereof of the present disclosure comprises a light chain CDR3 (L-CDR3) which comprises or consists of the following sequence:

(SEQ ID NO: 6)
N term-SQSTX₁₃X₁₄PX₁₅T-C term

wherein X₁₃ is H, A or E; X₁₄ is V, E, S, D or Y; and X₁₅ is F, S, T or Y. In some embodiments X₁₃ is H, X₁₄ is E and X₁₅ is F.

In some embodiments, the antibody or antigen-binding fragment or derivative thereof of the present disclosure comprises: a) a VH sequence as set forth in Table 1, or a variant sequence thereof which differs by only one or two amino acids, or which has at least or about 85% sequence identity thereto; and/or b) a VL sequence as set forth in Table 2, or a variant sequence thereof which differs by only one or two amino acids, or which has at least or about 85% sequence identity thereto.

In some embodiments, the antibody or antigen-binding fragment or derivative thereof of the present disclosure comprises: a) a VH region having the sequence set forth in any one of SEQ ID NOs: 7, 151 and 153-157, or a variant sequence thereof which differs by only one or two amino acids, or which has at least or about 85% sequence identity thereto; and/or b) a VL region having the sequence set forth in any one of SEQ ID NOs: 34 and 159, or a variant sequence thereof which differs by only one or two amino acids, or which has at least or about 85% sequence identity thereto.

In some embodiments, the antibody or antigen-binding fragment or derivative thereof of the present disclosure comprises: a) a VH region having the sequence of 15A4-12-VHA (SEQ ID NO: 151), 15A4-146-VHA (SEQ ID NO: 7), 15A4-4341-VHA (SEQ ID NO: 153), 15A4-4341-VHB (SEQ ID NO: 154), 15A4-4341-VHC (SEQ ID NO: 155), 15A4-4349-VHA (SEQ ID NO: 156) or 15A04-4349-VHB (SEQ ID NO: 157) or a variant sequence thereof which differs by only one or two amino acids, or which has at least or about 85% sequence identity thereto; and/or b) a VL region having the sequence of 15A4-230-VLA (SEQ ID NO: 34) or 15A4-218-VLA (SEQ ID NO: 159), or a variant sequence thereof which differs by only one or two amino acids, or which has at least or about 85% sequence identity thereto.

In one embodiment, the antibody or antigen-binding fragment or derivative thereof of the present disclosure comprises a VH region comprising or consisting of the sequence of 15A4-146-VHA (SEQ ID NO: 7) and/or a VL region comprising or consisting of the sequence of 15A4-230-VLA (SEQ ID NO: 34).

In some embodiments, the antibody or antigen-binding fragment or derivative thereof of the present disclosure comprises: six CDR amino acid sequences selected from: a) a combination of a heavy chain CDR1, CDR2, and CDR3 as set forth in Table 3, or a variant sequence thereof which differs by only one or two amino acids, or which has at least or about 85% sequence identity thereto; and b) a combination of a light chain CDR1, CDR2, and CDR3 as set forth in Table 4, or a variant sequence thereof which differs by only one or two amino acids, or which has at least or about 85% sequence identity thereto.

In some embodiments, the antibody or antigen-binding fragment or derivative thereof of the present disclosure comprises: six CDR amino acid sequences selected from: SEQ ID NOs: 79-81 and 101-103, or a variant sequence thereof which differs by only one or two amino acids, or which has at least or about 85% sequence identity thereto.

In some embodiments, the antibody or antigen-binding fragment or derivative thereof of the present disclosure comprises: six CDR amino acid sequences selected from: SEQ ID NOs: 1-6, or a variant sequence thereof which differs by only one or two amino acids, or which has at least or about 85% sequence identity thereto. In some embodiments, the antibody or antigen-binding fragment or derivative thereof for use in the methods of the present disclosure comprises: a VH comprising or consisting of the amino acid sequence set forth in any one of SEQ ID NOs: 7-22 or 25-33, or a variant sequence thereof which differs by only one or two amino acids, or which has at least or about 85% sequence identity thereto; and/or a VL comprising or consisting of the amino acid sequence set forth in any one of SEQ ID NOs: 34-52 or 54-78, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 7, or a variant sequence thereof which differs by only one or two amino acids, or which has at least or about 85% sequence identity thereto; and/or a VL comprising or consisting of the amino acid sequence set forth in SEQ ID NO: 34, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 12, or a variant sequence thereof which differs by only one or two amino acids, or which has at least or about 85% sequence identity thereto; and/or a VL comprising or consisting of the amino acid sequence set forth in SEQ ID NO: 65, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 14, or a variant sequence thereof which differs by only one or two amino acids, or which has at least or about 85% sequence identity thereto; and/or a VL comprising or consisting of the amino acid sequence set forth in SEQ ID NO: 65, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 17, or a variant sequence thereof which differs by only one or two amino acids, or which has at least or about 85% sequence identity thereto; and/or a VL comprising or consisting of the amino acid sequence set forth in SEQ ID NO: 74, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 17, or a variant sequence thereof which differs by only one or two amino acids, or which has at least or about 85% sequence identity thereto; and/or a VL comprising or consisting of the amino acid sequence set forth in SEQ ID NO: 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 32, or a variant sequence thereof which differs by only one or two amino acids, or which has at least or about 85% sequence identity thereto; and/or a VL comprising or consisting of the amino acid sequence set forth in SEQ ID NO: 65, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 7, or a variant sequence thereof which differs by only one or two amino acids, or which has at least or about 85% sequence identity thereto; and/or a VL comprising or consisting of the amino acid sequence set forth in any one of SEQ ID NOs: 34, 65, 74, and 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 12, or a variant sequence thereof which differs by only one or two amino acids, or which has at least or about 85% sequence identity thereto; and/or a VL comprising or consisting of the amino acid sequence set forth in any one of SEQ ID NOs: 34, 65, 74, and 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 14, or a variant sequence thereof which differs by only one or two amino acids, or which has at least or about 85% sequence identity thereto; and/or a VL comprising or consisting of the amino acid sequence set forth in any one of SEQ ID NOs: 34, 65, 74, and 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 17, or a variant sequence thereof which differs by only one or two amino acids, or which has at least or about 85% sequence identity thereto; and/or a VL comprising or consisting of the amino acid sequence set forth in any one of SEQ ID NOs: 34, 65, 74, and 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 32, or a variant sequence thereof which differs by only one or two amino acids, or which has at least or about 85% sequence identity thereto; and/or a VL comprising or consisting of the amino acid sequence set forth in any one of SEQ ID NOs: 34, 65, 74, and 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in any one of SEQ ID NOs: 7, 12, 14, 17, and 32, or a variant sequence thereof which differs by only one or two amino acids, or which has at least or about 85% sequence identity thereto; and/or a VL comprising or consisting of the amino acid sequence set forth in SEQ ID NO: 34, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in any one of SEQ ID NOs: 7, 12, 14, 17, and 32, or a variant sequence thereof which differs by only one or two amino acids, or which has at least or about 85% sequence identity thereto; and/or a VL comprising or consisting of the amino acid sequence set forth in SEQ ID NO: 65, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in any one of SEQ ID NOs: 7, 12, 14, 17, and 32, or a variant sequence thereof which differs by only one or two amino acids, or which has at least or about 85% sequence identity thereto; and/or a VL comprising or consisting of the amino acid sequence set forth in SEQ ID NO: 74, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in any one of SEQ ID NOs: 7, 12, 14, 17, and 32, or a variant sequence thereof which differs by only one or two amino acids, or which has at least or about 85% sequence identity thereto; and/or a VL comprising or consisting of the amino acid sequence set forth in SEQ ID NO: 75, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 7, or a variant sequence thereof which differs by only one or two amino acids, or which has at least or about 85% sequence identity thereto; and/or a VL comprising or consisting of an amino acid sequence shown in Table 2, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 12, or a variant sequence thereof which differs by only one or two amino acids, or which has at least or about 85% sequence identity thereto; and/or a VL comprising or consisting of an amino acid sequence shown in Table 2, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 14, or a variant sequence thereof which differs by only one or two amino acids, or which has at least or about 85% sequence identity thereto; and/or a VL comprising or consisting of an amino acid sequence shown in Table 2, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 17, or a variant sequence thereof which differs by only one or two amino acids, or which has at least or about 85% sequence identity thereto; and/or a VL comprising or consisting of an amino acid sequence shown in Table 2, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of the amino acid sequence set forth in SEQ ID NO: 32, or a variant sequence thereof which differs by only one or two amino acids, or which has at least or about 85% sequence identity thereto; and/or a VL comprising or consisting of an amino acid sequence shown in Table 2, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of an amino acid sequence shown in Table 1, or a variant sequence thereof which differs by only one or two amino acids, or which has at least or about 85% sequence identity thereto; and/or a VL comprising or consisting of the amino acid sequence set forth in SEQ ID NO: 34, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of an amino acid sequence shown in Table 1, or a variant sequence thereof which differs by only one or two amino acids, or which has at least or about 85% sequence identity thereto; and/or a VL comprising or consisting of the amino acid sequence set forth in SEQ ID NO: 65, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of an amino acid sequence shown in Table 1, or a variant sequence thereof which differs by only one or two amino acids, or which has at least or about 85% sequence identity thereto; and/or a VL comprising or consisting of the amino acid sequence set forth in SEQ ID NO: 74, or a variant sequence thereof which differs by only one or two amino acids, or which has at least or about 85% sequence identity thereto.

In some embodiments, the antibody or antigen-binding fragment or derivative thereof for use in the methods of the present disclosure comprises: a VH comprising or consisting of an amino acid sequence shown in Table 1, or a variant sequence thereof which differs by only one or two amino acids, or which has at least or about 85% sequence identity thereto; and/or a VL comprising or consisting of the amino acid sequence set forth in SEQ ID NO: 75, or a variant sequence thereof which differs by only one or two amino acids, or which has at least or about 85% sequence identity thereto.

In some embodiments, the antibody or antigen-binding fragment or derivative thereof of the present disclosure comprises: four FR amino acid sequences selected from: a) a combination of a heavy chain FR1, FR2, FR3 and FR4 as set forth in Table 5, or a variant sequence thereof which differs by only one or two amino acids, or which has at least or about 85% sequence identity thereto; and b) a combination of a light chain FR1, FR2, FR3 and FR4 as set forth in Table 6, or a variant sequence thereof which differs by only one or two amino acids, or which has at least or about 85% sequence identity thereto.

In some embodiments, the antibody or antigen-binding fragment or derivative thereof of the present disclosure comprises an immunoglobulin heavy chain constant domain selected from the group consisting of human IgG1, IgG2, IgG2A, IgG2B, IgG3, IgG4, IgA, IgM, IgD, and IgE constant domains. In some embodiments, the antibody or antigen-binding fragment or derivative thereof of the present disclosure comprises an immunoglobulin heavy chain constant domain which is IgG1. In some embodiments, the antibody or antigen-binding fragment or derivative thereof of the present disclosure comprises an immunoglobulin heavy chain constant domain which is IgG2. In some embodiments, the antibody or antigen-binding fragment or derivative thereof of the present disclosure comprises an immunoglobulin heavy chain constant domain which is IgG4.

In some embodiments, the antibody or antigen-binding fragment or derivative thereof of the present disclosure comprises an effector domain, comprises an Fc domain, and/or is selected from the group consisting of Fv, F(ab′)2, Fab′, dsFv, scFv, sc(Fv)2, and diabody fragments.

In some embodiments, the antibody or antigen-binding fragment or derivative thereof of the present disclosure comprises or consists of 15A4-12-VHA-230-VLA (SEQ ID NOs: 151/34, 15A4-4341-VHA-230-VLA (SEQ ID NOs: 153/34), 15A4-4341-VHB-230-VLA (SEQ ID NOs: 154/34), 15A4-4341-VHC-230-VLA (SEQ ID NOs: 155/34), 15A4-4349-VHA-230-VLA (SEQ ID NOs: 156/34), 15A4-4349-VHB-230-VLA (SEQ ID NOs: 157/34), 15A4-12-VHA-218-VLA (SEQ ID NOs: 151/159), 15A4-4341-VHA-218-VLA (SEQ ID NOs: 153/159), 15A4-4341-VHB-218-VLA (SEQ ID NOs: 154/159), 15A4-4341-VHC-218-VLA (SEQ ID NOs: 155/159), 15A4-4349-VHA-218-VLA (SEQ ID NOs: 156/159) or 15A4-4349-VHA-218-VLA (SEQ ID NOs: 157/159); or a variant sequence thereof which differs by only one, two or three amino acids, or which has at least or about 85% sequence identity thereto.

As used herein, references to A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26 antibodies, or antigen-binding fragments thereof, correspond to polypeptide sequences as defined in Tables 1 to 4 of the present disclosure.

In some embodiments, the antibody or antigen-binding fragment or derivative thereof of the present disclosure comprises or consists of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26 with respective sequences as provided herein; or a variant sequence thereof which differs by only one, two or three amino acids, or which has at least or about 85% sequence identity thereto. In some such embodiments, the antibody or antigen-binding fragment or derivative thereof does not comprise or consist of A02 or A03.

In some embodiments, the antibody or antigen-binding fragment or derivative thereof of the present disclosure comprises the VH or the VL polypeptides of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26.

In some embodiments, the antibody or antigen-binding fragment or derivative thereof of the present disclosure comprises the VH and the VL polypeptides of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26. In some such embodiments, the antibody or antigen-binding fragment or derivative thereof does not comprise the VH and VL polypeptides of A02 or A03.

In some embodiments, the antibody, or antigen-binding fragment or derivative thereof of the present disclosure comprises or consists of any one of heavy chain variable regions (VH) selected from the group consisting of: SEQ ID NO: 7, 8, 9, 10, 11, 12, 13, 14, 15, 16, 17, 18, 19, 20, 21, 22, 25, 26, 27, 28, 29, 30, 31, 32, 33; and/or wherein said antibody, or antigen-binding fragment comprises or consists of any one of light chain variable regions (VL) selected from the group consisting of: SEQ ID NO: 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 71, 72, 73, 74, 75, 76, 77, 78; or a variant sequence thereof which differs by only one, two or three amino acids, or which has at least or about 85% sequence identity thereto.

In some embodiments, the antibody, or antigen-binding fragment or derivative thereof of the present disclosure comprises or consists of any one of heavy chain variable regions (VH) selected from the group consisting of: SEQ ID NO: 7, 8, 9, 10, 11, 12, 13, 14, 15, 16, 17, 18, 19, 20, 21, 22, 25, 26, 27, 28, 29, 30, 31, 32, 33; and wherein said antibody, or antigen-binding fragment comprises or consists of any one of light chain variable regions (VL) selected from the group consisting of: SEQ ID NO: 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 71, 72, 73, 74, 75, 76, 77, 78.

In some embodiments, the antibody or antigen-binding fragment or derivative thereof of the present disclosure comprises the three heavy chain CDRs of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26.

In some embodiments, the antibody or antigen-binding fragment or derivative thereof of the present disclosure comprises the three light chain CDRs of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26.

In some embodiments, the antibody or antigen-binding fragment or derivative thereof of the present disclosure comprises the six CDRs of A01, B16, B01, B02, B03, B04, B05, B06, B07, B08, B09, B10, B11, B12, B13, B14, B15, B17, B18, B19, B20, B21, B22, A02, A03, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26.

In one embodiment, the antibody or antigen-binding fragment or derivative thereof of the present disclosure comprises one or more CDR amino acid sequences selected from SEQ ID NOs: 79-81 and 101-103, or a variant sequence thereof which differs by only one or two amino acids, or which has at least or about 85% sequence identity thereto.

In one embodiment, the antibody or antigen-binding fragment or derivative thereof of the present disclosure comprises one or more CDR amino acid sequences selected from SEQ ID NOs: 1-6, or a variant sequence thereof which differs by only one or two amino acids, or which has at least or about 85% sequence identity thereto.

In one embodiment, the antibody or antigen-binding fragment or derivative thereof of the present disclosure comprises the following CDRs: CDR-H1 comprising or consisting of the sequence set forth in SEQ ID NO: 79; CDR-H2 comprising or consisting of the sequence set forth in SEQ ID NO: 80; CDR-H3 comprising or consisting of the sequence set forth in SEQ ID NO: 81; CDR-L1 comprising or consisting of the sequence set forth in SEQ ID NO: 101; CDR-L2 comprising or consisting of the sequence set forth in SEQ ID NO: 102; and CDR-L3 comprising or consisting of the sequence set forth in SEQ ID NO: 103. In one embodiment, the antibody or antigen-binding fragment or derivative thereof of the present disclosure comprises the following CDRs: CDR-H1 comprising or consisting of the sequence set forth in SEQ ID NO: 1; CDR-H2 comprising or consisting of the sequence set forth in SEQ ID NO: 2; CDR-H3 comprising or consisting of the sequence set forth in SEQ ID NO: 3; CDR-L1 comprising or consisting of the sequence set forth in SEQ ID NO: 4; CDR-L2 comprising or consisting of the sequence set forth in SEQ ID NO: 5; and CDR-L3 comprising or consisting of the sequence set forth in SEQ ID NO: 6.

In one embodiment, the antibody or antigen-binding fragment or derivative thereof of the present disclosure comprises the following CDRs: CDR-H1 comprising or consisting of the sequence set forth in SEQ ID NO: 82; CDR-H2 comprising or consisting of the sequence set forth in SEQ ID NO: 80; CDR-H3 comprising or consisting of the sequence set forth in SEQ ID NO: 81; CDR-L1 comprising or consisting of the sequence set forth in SEQ ID NO: 132; CDR-L2 comprising or consisting of the sequence set forth in SEQ ID NO: 105; and CDR-L3 comprising or consisting of the sequence set forth in SEQ ID NO: 106.

In one embodiment, the antibody or antigen-binding fragment or derivative thereof of the present disclosure comprises the following CDRs: CDR-H1 comprising or consisting of the sequence set forth in SEQ ID NO: 82; CDR-H2 comprising or consisting of the sequence set forth in SEQ ID NO: 92; CDR-H3 comprising or consisting of the sequence set forth in SEQ ID NO: 81; CDR-L1 comprising or consisting of the sequence set forth in SEQ ID NO: 132; CDR-L2 comprising or consisting of the sequence set forth in SEQ ID NO: 105; and CDR-L3 comprising or consisting of the sequence set forth in SEQ ID NO: 106.

In one embodiment, the antibody or antigen-binding fragment or derivative thereof of the present disclosure comprises the following CDRs: CDR-H1 comprising or consisting of the sequence set forth in SEQ ID NO: 82; CDR-H2 comprising or consisting of the sequence set forth in SEQ ID NO: 94; CDR-H3 comprising or consisting of the sequence set forth in SEQ ID NO: 81; CDR-L1 comprising or consisting of the sequence set forth in SEQ ID NO: 128; CDR-L2 comprising or consisting of the sequence set forth in SEQ ID NO: 105; and CDR-L3 comprising or consisting of the sequence set forth in SEQ ID NO: 106.

In one embodiment, the antibody or antigen-binding fragment or derivative thereof of the present disclosure comprises the following CDRs: CDR-H1 comprising or consisting of the sequence set forth in SEQ ID NO: 82; CDR-H2 comprising or consisting of the sequence set forth in SEQ ID NO: 94; CDR-H3 comprising or consisting of the sequence set forth in SEQ ID NO: 81; CDR-L1 comprising or consisting of the sequence set forth in SEQ ID NO: 132; CDR-L2 comprising or consisting of the sequence set forth in SEQ ID NO: 105; and CDR-L3 comprising or consisting of the sequence set forth in SEQ ID NO: 117.

In one embodiment, the antibody or antigen-binding fragment or derivative thereof of the present disclosure comprises the following CDRs: CDR-H1 comprising or consisting of the sequence set forth in SEQ ID NO: 82; CDR-H2 comprising or consisting of the sequence set forth in SEQ ID NO: 95; CDR-H3 comprising or consisting of the sequence set forth in SEQ ID NO: 81; CDR-L1 comprising or consisting of the sequence set forth in SEQ ID NO: 132; CDR-L2 comprising or consisting of the sequence set forth in SEQ ID NO: 105 and CDR-L3 comprising or consisting of the sequence set forth in SEQ ID NO: 106.

In some embodiments, the antibody or antigen-binding fragment or derivative thereof of the present disclosure further comprises a signal peptide, e.g., a signal peptide of SEQ ID NO: 176, at the N-terminus. In some embodiments, the signal peptide is cleaved from the mature and/or purified protein.

In some embodiments, the composition of the present disclosure comprises a nucleic acid molecule that encodes: a) a polypeptide comprising or consisting of an amino acid sequence listed in Table 1 and/or Table 2; b) a polypeptide comprising or consisting of an amino acid sequence with at least or about 85% identity to an amino acid sequence listed in Table 1 and/or Table 2; and/or c) a monoclonal antibody or antigen-binding fragment or derivative thereof, as described herein. In some embodiments, the nucleic acid molecule further comprises a sequence (e.g., SEQ ID NO: 158) encoding a signal peptide at the 5′ end. In some embodiments, the nucleic acid molecule has the nucleotide sequence set forth in any one of SEQ ID NOs: 184 to 355. In some embodiments, the composition comprises a vector comprising the isolated nucleic acid as described herein. In some embodiments, the composition comprises a host cell comprising the isolated nucleic acid or the vector as described herein.

In a second broad aspect, there are provided humanized antibodies or antigen-binding fragments or derivatives thereof which are specific for the GluN1 subunit of the NMDAR, wherein said antibody, fragment or derivative competes for binding with an antibody produced by a deposited hybridoma which is 15A4B2E5, 15A4B2F3, 15A4B2, 6C9A3, 6C9A3F4, or 6C9A3F6.

The deposited hybridoma are as described in WO2014/187879. The hybridomas 15A4B2E5, 15A4B2F3, 15A4B2, 6C9A3, 6C9A3F4 and 6C9A3F6 were deposited at the DSMZ (Leibniz-Institut DSMZ-Deutsche Sammlung von Mikroorganismen und Zellkulturen GmbH, InhoffenstraBe 7B, 38124 Braunschweig, Germany) and have been given the accession numbers DSM ACC3203 (received on May 29, 2013), DSM ACC3217 (received on Sep. 25, 2013), DSM ACC3216 (received on Sep. 25, 2013), DSM ACC3214 (received on Sep. 25, 2013), DSM ACC3200 (received on May 29, 2013) and DSM ACC3215 (received on Sep. 25, 2013), respectively. The depositors are PAION Deutschland GmbH, Martinstrasse 10-12, D-52062 Aachen, Germany and INSERM, 101 rue de Tolbiac, F-75013 Paris, France, acting through its unit INSERM-UCBN, UMR-S U919, GIP CYCERON, Boulevard Henri Becquerel, BP 5229-14074 Caen Cedex, France. All of these hybridomas produce antibodies of the isotype lgG2 (kappa light chains). Suitable culturing conditions are as follows. Culture medium: DMEM (4.5 g/l with Glutamine & sodium pyruvate) ref PAA E15-843 supplemented with 10% FCS; culture in incubator 37<C, 5 to 10% C0₂; thaw cells in water bath at 37<C for few seconds and transfer ice cube into the 30 ml pre-heated medium (37<C); after centrifugation (7 min, 1 200 rpm) resuspend cells in 10 ml pre-heated culture medium (37<C) and transfer in T25 cm² flask; put the flask horizontally in the incubator; split cells 1:5 Monday and Wednesday; 1:10 Friday. Gentamycin 0.1 mg/ml may be used.

In some such embodiments, the humanized antibody, fragment or derivative competes for binding with an antibody which is glunomab, i.e., the mouse antibody 15A4-B2-E5 (SEQ ID NOs: 23/53).

In a third broad aspect, there are provided nucleic acids encoding the humanized antibodies or antigen-binding fragments or derivatives thereof according to the disclosure. In some embodiments, there are provided nucleic acids encoding the humanized antibodies or antigen-binding fragments or derivatives thereof according to the disclosure, wherein the nucleic acid molecule has the nucleotide sequence set forth in any one of SEQ ID NOs: 184 to 355. In some embodiments, there is provided an expression cassette or vector comprising the nucleic acid of the disclosure. Such expression cassettes or vectors may comprise a promoter operatively connected with the nucleic acid for expression thereof. In some embodiments, there is provided a host cell comprising the nucleic acid, the expression cassette or the vector of the disclosure.

In a fourth broad aspect, there are provided pharmaceutical compositions comprising the humanized antibodies or antigen-binding fragments or derivatives thereof according to the disclosure, and a pharmaceutically acceptable carrier, diluent or excipient. In some embodiments, there is provided a pharmaceutical composition comprising the nucleic acid, the expression cassette, the vector, or the host cell of the disclosure. Pharmaceutical compositions may be suitable for administration by injection (e.g., subcutaneously, intramuscularly, intravenously, intraperitoneally), nasally, topically, or orally.

In a fifth broad aspect, there are provided methods of treating or preventing a neurological, neurovascular or neurodegenerative disorder in a subject comprising administering the humanized antibodies, antigen-binding fragments or derivatives thereof or pharmaceutical compositions thereof according to the disclosure to the subject, such that the neurological, neurovascular or neurodegenerative disorder is treated or prevented in the subject. Examples of neurological, neurovascular or neurodegenerative disorders which may be treated or prevented in accordance with the methods of the disclosure include, without limitation: multiple sclerosis, thrombotic disorder, stroke, intracerebral hemorrhage, intracerebral bleeding, transient ischemic attack, epilepsy, temporal lobe epilepsy, amyotrophic lateral sclerosis, brain tumor, Parkinson's disease, Alzheimer's disease, Huntington's disease, brain oedema, age-related macular degeneration, CNS complication resulting from parasitic, bacterial, fungal or viral infection, meningitis, encephalitis, traumatic brain injury, spinal cord injury, intracranial lesion, intravertebral lesion, ischemic disorder, or damage of the optic nerve after acute vascular occlusion or in glaucoma.

In some embodiments, there are provided methods of diagnosis, prognosis and/or monitoring of a neurological, neurovascular or neurodegenerative disorder in a subject, comprising administering the humanized antibodies, antigen-binding fragments or derivatives thereof or pharmaceutical compositions thereof according to the disclosure to the subject.

In some embodiments, there are provided methods of using the humanized antibodies, antigen-binding fragments or derivatives thereof or pharmaceutical compositions thereof as neuroprotectants in a subject.

As used herein, the terms “neuroprotectant” or “neuroprotective agent” may refer, in particular, to a compound (e.g. an antibody, antigen-binding fragment or derivative thereof according to the disclosure) or composition thereof, that protects (prevents damage(s) occurring to) the nervous system, particularly the brain, the spinal cord or cells thereof, especially within the context of an ongoing acute or chronic disorder. Brain cells which are particularly considered, include neurons and glial cells. In a non-exhaustive manner, a neuroprotectant aims to preserve the neuronal structure and function through e.g. reduction of inflammation, inhibition of excitotoxicity, prevention of the oxidative stress, maintenance of the mitochondrial function and/or blocking of apoptosis.

In some embodiments, there are provided methods of using the humanized antibodies, antigen-binding fragments or derivatives thereof or pharmaceutical compositions thereof in a subject having an acute or chronic disorder; for example a subject having an inflammatory disorder.

In some embodiments of methods of the present disclosure, the subject is a mammal, e.g., a human or an animal, e.g., a pet (dog, cat, mouse, rat, etc.), livestock (cow, sheep, goat, horse, etc.), an animal model of a neurological, neurovascular or neurodegenerative disease, etc. Since the disclosure provides antibodies which are humanized, it is expected that in certain embodiments, the subject is a human.

In methods of the present disclosure, the antibodies or the pharmaceutical compositions thereof may be administered using any suitable route of administration. Non-limiting routes of administration include parenteral, e.g., intravenous, intradermal, subcutaneous, intraperitoneal; oral, e.g., inhalation, swallowing; transdermal or topical; transmucosal; or rectal administration.

In certain aspects, there are provided therapeutic and prophylactic methods as described in detail herein.

In another aspect, there are provided kits comprising a humanized antibody or antigen-binding fragment or derivative thereof, or pharmaceutical composition thereof according to the disclosure, and instructions for methods of use thereof for treatment, prevention, diagnosis, prognosis and/or monitoring of a neurological and/or neurodegenerative disorder.

In another aspect, there are provided kits comprising a nucleic acid encoding the antibody or antigen-binding fragment or derivative thereof, or expression cassette or vector comprising the nucleic acid, or host cell, and instructions for methods of use thereof for treatment, prevention, diagnosis, prognosis and/or monitoring of a neurological and/or neurodegenerative disorder.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIG. 1 shows 15A4-12-VHA, which is a CDR-grafted version of 15A4-B2-E5 VH using human germline IGHV1-2*01 as human germline acceptor. The sequences of the CDRs as defined by the Kabat nomenclature are shown in bold and underlined. Residues that differ between humanized version A and human germline IGHV1-2*01 are double underlined.

FIG. 2 shows 15A4-146-VHA, which is a CDR-grafted version of 15A4-B2-E5 VH using human germline IGHV1-46*01 as human germline acceptor. The sequences of the CDRs as defined by the Kabat nomenclature are shown in bold and underlined. Residues that differ between humanized version A and human germline IGHV1-46*01 are double underlined.

FIGS. 3A-3C show CDR-grafted versions of 15A4-B2-E5 VH using human germline IGHV4-34*01 as human germline acceptor. FIG. 3A shows Humanized version A (15A4-4341-VHA); FIG. 3B shows Humanized version B (15A4-4341-VHB); and FIG. 3C shows Humanized version C (15A4-4341-VHC). The sequences of the CDRs as defined by the Kabat nomenclature are shown in bold and underlined. Residues that differ between 15A4-4341-VHA (SEQ ID NO: 153) and human germline IGHV4-34*01 are double underlined in FIG. 3A. The residue that differs between Humanized version A and Humanized version B in CDR2 is double underlined in FIG. 3B. The residue that differs between Humanized version B and Humanized version C in FR3 is double underlined in FIG. 3C.

FIGS. 4A-4B show CDR-grafted versions of 15A4-B2-E5 VH using human germline IGHV4-34*09 as human germline acceptor. FIG. 4A shows Humanized version A (15A4-4349-VHA), and FIG. 4B shows Humanized version B (15A4-4349-VHB). The sequences of the CDRs as defined by the Kabat nomenclature are shown in bold and underlined. Residues that differ between 15A4-4349-VHA (SEQ ID NO: 156) and human germline IGHV4-34*09 are double underlined in FIG. 4A. The residue that differs between Humanized version A and Humanized version B in FR3 is double underlined in FIG. 4B.

FIG. 5 shows 15A4-230-VLA, which is a CDR-grafted version of 15A4-B2-E5 VL using human germline IGKV2-30*02 as human germline acceptor. The sequences of the CDRs as defined by the Kabat nomenclature are shown in bold and underlined. Residues that differ between 15A4-230-VLA and human germline IGKV2-30*02 are double underlined.

FIG. 6 shows 15A4-218-VLA, which is a CDR-grafted version of 15A4-B2-E5 VL using human germline IGKV2-18*01 as human germline acceptor. The sequences of the CDRs as defined by the Kabat nomenclature are shown in bold and underlined. Residues that differ between 15A4-218-VLA (SEQ ID NO: 159) and human germline IGKV2-18*01 are double underlined.

FIGS. 7A-7B show the sequences of the VH and VL polypeptide chains of the mouse antibody 15A4-B2-E5, respectively (SEQ ID NOs: 23 and 53, respectively). The sequences of the CDRs as defined by the Kabat nomenclature are shown in bold and underlined.

FIG. 8 is a schematic diagram showing the affinity maturation program used to screen the A01 variant combinatorial phage display library.

FIG. 9 is a graph showing a comparison of expected (dotted line) and observed (histogram) number of mutations per clone of the A01 variants library. Both representations had similar distributions with a peak centered between 4 to 6 mutations per clone.

FIG. 10 shows biolayer interferometry (BLI) sensorgrams of Fabs binding to the epitope peptide.

FIG. 11 shows experimental design of antibody fragmentation study upon acid conditions.

FIG. 12 shows experimental design of ELISA on ATD protein.

FIG. 13 shows experimental design of BLI assay on epitope peptide. In \textcircled{1} the antigen represented by the epitope peptide was fixed to the biosensor precoated with Streptavidin (SA); \textcircled{2} then the antibodies were added individually to the solution and the kinetics of binding were recorded until reaching a plateau, allowing calculation of Kₒₙ; \textcircled{3} then, the unbound antibodies were washed away and dissociation kinetics were recorded until reaching a plateau, allowing calculation of k_{off}.

FIG. 14 shows experimental design of ELISA for binding to DNA, insulin and (3-galactosidase.

FIG. 15 shows results from testing 5 selected A01 variants for target binding, in ELISA on human ATD23-371_TwiStreptag (batch #6). The following isotypes were tested: A01: human IgG1; C28: human IgG1 and human IgG4; D11, D14, D15 and D17: human IgG1-LALA mutated.

FIG. 16 shows results from testing 5 selected A01 variants for target binding, in BLI assay on epitope peptide (GPS_22474). The following isotypes were tested: A01: human IgG1; C28: human IgG1 and human IgG4; D11, D14, D15 and D17: human IgG1-LALA mutated.

FIG. 17 shows results from testing 5 selected A01 variants for polyreactivity, in ELISA on insulin, DNA and β-galactosidase (OD 450 nm). The following isotypes were tested: A01: human IgG1; C28 human IgG1 and human IgG4; D11, D14, D15 and D17: human IgG1-LALA mutated.

FIG. 18 shows experimental design of testing in the NMDA lesion model in mice.

FIG. 19 shows representation of lesion volume measurement in a mouse model of NMDA-induced brain lesion after treatment with recombinant t-PA (rt-PA) in the presence or absence of the indicated test proteins. Quantifications were done in NMDA-injured mice treated with vehicle (NaCl 0.9% solution) alone, rt-PA alone or co-treated with the A01 antibody or A01 variants (14 groups; n=8 per group; statistical analyses with Kruskal-Wallis test; *p<0.05; **p<0.01; p<0.001; ****p<0.0001). The A01 antibody and all its variants were tested as human IgG1 isotype.

FIG. 20 shows the experimental design (A) and comparative results between D11 antibody and glunomab treatment (B) in NMDA-injured mice. The quantifications were done in NMDA-injured mice treated with vehicle, with rtPA, with rtPA+glunomab (10 mg/kg), or with rtPA+D11 antibody (5 mg/kg). n=8 per group; statistical analyses with Kruskal-Wallis test: **p<0.01, ****p<0.0001.

### DETAILED DESCRIPTION

The present disclosure provides humanized antibodies specific for the GluN1 subunit of the NMDAR and antigen-binding fragments or derivatives thereof, as well as pharmaceutical compositions and methods of use thereof for the treatment and prevention of neurological and/or neurodegenerative disorders.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The articles “a” and “an” are used herein to refer to one or to more than one (i.e. to at least one) of the grammatical object of the article. By way of example, “an element” means one element or more than one element.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

As used herein, the term “antibody” refers to a protein comprising at least two heavy chains and two light chains connected by disulfide bonds. The term “antibody” includes naturally occurring antibodies as well as all recombinant forms of antibodies, e.g., antibodies expressed in prokaryotes, unglycosylated antibodies, humanised antibodies, and chimeric antibodies. Each heavy chain consists of a heavy chain variable region (VH) and a heavy chain constant region (CH). Each light chain consists of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. The term “antibody” according to the disclosure also includes unusual antibodies such as heavy chain antibodies, i.e., antibodies only composed of one or more, in particular two heavy chains, and nanobodies, i.e. antibodies only composed of a single monomeric variable domain.

The terms “CDR”, and its plural “CDRs,” refer to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5^{th} Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

As used herein, the term “Fc region” is used to describe a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present disclosure include, without limitation, human IgG1, IgG2 (IgG2A, IgG2B), IgG3 and IgG4.

As used herein, the term “anti-NMDA receptor antibody”, abbreviated “anti-NMDAR antibody”, is used to encompass antibodies that are specific for the NMDA receptor (NMDAR). In certain embodiments, the term includes antibodies specific for the GluN1 subunit of the NMDAR, i.e., anti-GluN1 antibodies. In certain embodiments, the term includes anti-GluN1 antibodies that block or inhibit interaction between the NMDAR and t-PA and/or activation of the NMDAR by t-PA. In certain embodiments, the term includes antibodies that selectively inhibit some but not all functions of the NMDAR.

As used herein, the term “protein” refers to a molecular chain of amino acids or a complex of more than one amino acid chain. A protein may contain any of the naturally occurring amino acids as well as artificial amino acids and may be of biologic or synthetic origin. A protein may be modified, naturally (post-translational modifications) or synthetically, by e.g. glycosylation, amidation, carboxylation and/or phosphorylation. A protein comprises at least two amino acids, but does not have to be of any specific length; this term does not include any size restrictions. In the present application, the terms “protein”, “polypeptide” and “peptide” are used interchangeably.

A “fragment or derivative” of an antibody is a protein or glycoprotein which is derived from said antibody and is capable of binding to the same antigen, in particular to the same epitope as the antibody. Thus, a fragment or derivative of an antibody herein generally refers to an antigen-binding and functional fragment or derivative. In some embodiments, the fragment or derivative of an antibody comprises a heavy chain variable region. It has been shown that the antigen-binding function of an antibody can be performed by fragments of a full-length antibody or fragments of derivatives thereof. Examples of fragments or derivatives of an antibody include, without limitation (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments, consisting of the variable region and the first constant domain CH1 of the heavy chain; (iv) Fv fragments, consisting of the heavy chain and light chain variable regions of a single arm of an antibody; (v) scFv fragments, Fv fragments consisting of a single polypeptide chain; (vi) (Fv)₂ fragments, consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecularly but not intramolecularly. These antibody fragments and derivatives are obtained using conventional techniques known to those skilled in the art.

A target amino acid sequence is “derived” from a reference amino acid sequence, for example, if the target amino acid sequence shares a homology or identity over its entire length with a corresponding part of the reference amino acid sequence of 60% or more, 70% or more, 75% or more, 80% or more, 85% or more, 90% or more, 93% or more, 95% or more, 97% or more, 98% or more, or 99% or more. The “corresponding part” or “corresponding framework region” means that, for example, framework region 1 of a heavy chain variable region (FRH1) of a target antibody corresponds to FRH1 of the reference antibody. The same is true, for example, for FRH2, FRH3, FRH4, FRL1, FRL2, FRL3 and FRL4. In some embodiments, a target amino acid sequence which is “derived” from a reference amino acid sequence is 100% identical, over its entire length with a corresponding part of the reference amino acid sequence.

In some embodiments, an antibody or antigen-binding fragment or derivative thereof is a homodimer. In some embodiments, an antibody or antigen-binding fragment or derivative thereof comprises two heavy chain variable regions that are identical to each other and/or two light chain variable regions that are identical to each other.

As used herein, the terms “K_{D}”, “Kd” or “K_{d}” are intended to refer to the dissociation equilibrium constant of a particular antibody-antigen interaction. The binding affinity of antibodies of the disclosure may be measured or determined by standard antibody-antigen assays, for example, competitive assays, saturation assays, or standard immunoassays such as ELISA or RIA.

As used herein, the term “specific for” means that a polypeptide (such as an antibody) specifically interacts with a given target(s). Specific binding is believed to be effected by specific motifs in the amino acid sequence of a polypeptide. Thus, binding is achieved as a result of their primary, secondary and/or tertiary structure as well as the result of secondary modifications of said structures. The specific interaction of the target-interaction-site with its specific target may result in a simple binding of said site to the target, or may alternatively or additionally result in the initiation of a signal, e.g. due to the induction of a change of the conformation of the target, an oligomerization of the target, or may block the target from performing another activity, such as binding to an endogenous molecule. In some embodiments, the K_{D} for the target or antigen to which the polypeptide binds specifically is more than 2-fold, more than 5-fold, more than 10-fold, more than 20-fold, more than 50-fold, more than 100-fold, more than 200-fold, more than 500-fold, or more than 1000-fold lower than the K_{D} for another target or antigen to which the polypeptide does not bind specifically. The terms “(specifically) binds to”, “(specifically) recognizes”, “specific for”, “is (specifically) directed to”, and “(specifically) reacts with” are used interchangeably herein.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “preventing” is art-recognized, and when used in relation to a condition, such as a neurological, neurovascular or neurodegenerative disease such as stroke, multiple sclerosis (MS), Parkinson's disease, etc. is well understood in the art, and includes administration of a treatment, e.g., a composition which reduces the frequency of, or delays the onset of, symptoms of a medical condition in a subject relative to a subject which does not receive the treatment. Thus, prevention of a neurological, neurovascular or neurodegenerative disorder includes, for example, reducing neuronal cell death or cognitive symptoms in a population of patients receiving a prophylactic treatment relative to an untreated control population, and/or delaying the appearance of detectable symptoms in a treated population versus an untreated control population, e.g., by a statistically and/or clinically significant amount.

As used herein, “subject” refers to any healthy animal, mammal or human, or any animal, mammal or human afflicted with a neurological, neurovascular or neurodegenerative disorder or at risk therefor. The term “subject” is interchangeable with “patient”. The term “non-human animal” includes all vertebrates, e.g., mammals and non-mammals, such as non-human primates, sheep, dog, cow, chickens, amphibians, reptiles, etc. In certain embodiments, the subject is a human.

### Antigen-Binding Proteins

Antigen-binding proteins for use in the methods of the disclosure are proteins that bind specifically to the NMDAR and block or inhibit interaction between the NMDAR and t-PA. The antigen-binding proteins for use in the methods of the present disclosure can take any one of many forms of antigen-binding proteins known in the art. In various embodiments, the antigen-binding proteins of the present disclosure take the form of an antibody, or antigen-binding antibody fragment or derivative, or an antibody protein product.

In various embodiments of the present disclosure, the antigen-binding protein comprises, consists essentially of, or consists of an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” chain (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three CDRs, is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

Unless otherwise specified here within, antibody or antibodies broadly encompass naturally-occurring forms of antibodies (e.g. IgG, IgA, IgM, IgE, IgD) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An antibody refers to a glycoprotein comprising at least two heavy (H) chains and two light (L) chains inter-connected by disulfide bonds, or an antigen binding portion thereof. Each heavy chain is comprised of a heavy chain variable region (VH) and a heavy chain constant region. The heavy chain constant region is comprised of three domains, CH1, CH2 and CH3. Each light chain is comprised of a light chain variable region (VL) and a light chain constant region. The light chain constant region is comprised of one domain, CL. The VH and VL can be further subdivided into regions of hypervariability, termed complementarity determining regions (CDR), interspersed with regions that are more conserved, termed framework regions (FR). Each VH and VL is composed of three CDRs and four FRs, arranged from the N-terminus to the C-terminus in the following order: FR1, CDR1, FR2, CDR2, FR3, CDR3, FR4. FRs residues are those variable-domain residues other than the hypervariable residues as herein indicated. VH and VL contain a binding domain that interacts with an antigen.

The general structure and properties of CDRs of antibodies have been described in the art. Briefly, in an antibody scaffold, the CDRs are embedded within a framework in the VH and VL where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a FR (designated framework regions 1-4, FR1, FR2, FR3, and FR4, by Kabat et al., 1991; see also Chothia and Lesk, 1987, supra). In a related embodiment, the residues of the framework are substituted with other amino acids. The heavy chain FRs which can be substituted lie within regions designated H-FR1, H-FR2, H-FR3 and H-FR4, which surround the heavy chain CDR residues, and the residues of the light chain FRs which can be substituted lie within the regions designated L-FR1, L-FR2, L-FR3 and L-FR4, which surround the light chain CDR residues. An amino acid within the FR may be replaced, for example, with any suitable amino acid identified in a human framework or human consensus framework.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The CL can be, for example, a kappa- or lambda-type CL, e.g., a human kappa- or lambda-type CL. The heavy chain constant region can be, for example, an alpha-, delta-, epsilon-, gamma-, or mu-type heavy chain constant region, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDRs from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production such as, for example, the international ImMunoGeneTics information System® (IMGT®).

Antibody as used herein also includes antigen-binding portion of an antibody. The antigen-binding portion refers to one or more fragments of an antibody that retain the ability to specifically bind to an antigen (e.g., a ganglioside). It has been shown that the antigen-binding function of an antibody can be performed by fragments of a full-length antibody. Examples of binding fragments encompassed within the antigen-binding portion of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; Huston et al. (1988) Proc. Natl. Acad. Sci. USA 85:5879-5883; and Osbourn et al. 1998, Nature Biotechnology 16: 778). Such single chain antibodies are also intended to be encompassed within the antigen-binding portion of an antibody.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab fragment or a F(ab′)2 fragment.

The architecture of antibodies has been exploited to create a growing range of alternative antibody formats that spans a molecular-weight range of at least or about 12-150 kDa and has a valency (n) range from monomeric (n=1), to dimeric (n=2), to trimeric (n=3), to tetrameric (n=4), and potentially higher; such alternative antibody formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions and stabilize the molecule to generate a scFv (single chain fragment variable) fragment, or the constant (C) domains are added to the V regions to generate a Fab fragment (fragment, antigen-binding). Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain antibodies as well as single domain antibodies (sdAb). The building block that is most frequently used to create novel antibody formats is the scFv fragment, which comprises V domains from the heavy and light chain (VH and VL domain) linked by a peptide linker of ~15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a triabody; a tetrabody; bispecific or trispecific antibodies, and the like. Bispecific antibodies can be divided into five major classes: BsIgG, appended IgG, bispecific antibody (BsAb) fragments, bispecific fusion proteins, and BsAb conjugates. See, e.g., Spiess et al., Molecular Immunology 67(2) Part A: 97-106 (2015).

In various aspects, the antigen-binding protein for use in the methods of the present disclosure comprises, consists essentially of, or consists of any one of these antibody protein products. In various aspects, the antigen-binding protein of the present disclosure comprises, consists essentially of, or consists of any one of a Fab VHH/VH, Fv fragment, ds-scFv, scFab, Fv, F(ab′)2, Fab′, dsFv, scFv, sc(Fv)2, dimeric antibody, multimeric antibody (e.g., a diabody, triabody, tetrabody), miniAb, peptibody VHH/VH of camelid heavy chain antibody, and sdAb.

In various instances, the antigen-binding protein for use in the methods of the present disclosure is an antibody protein product in monomeric form, or polymeric, oligomeric, or multimeric form.

In certain aspects, provided herein is a monoclonal antibody, or antigen-binding fragment or derivative thereof, wherein the monoclonal antibody specifically binds to the the GluN1 subunit of the NMDAR.

In various instances, the antigen-binding protein for use in the methods of the present disclosure is a multispecific antibody specific for one or more additional antigen or epitope.

In various embodiments, an anti-GluN1 antibody or antibody fragment or derivative thereof is a human antibody, a humanized antibody, a chimeric antibody, a monoclonal antibody, a recombinant antibody, an antigen-binding antibody fragment, a single chain antibody, a camelid antibody, a llama antibody, a monomeric antibody, a diabody, a triabody, a tetrabody, Fv, F(ab′)2, Fab′, dsFv, scFv, sc(Fv)2, an IgG1 antibody, an IgG2 antibody, an IgG3 antibody, or an IgG4 antibody.

In certain embodiments, an antibody of the present disclosure is a humanized monoclonal antibody, or antigen-binding fragment or derivative thereof, wherein the antibody or antigen-binding fragment or derivative thereof comprises a) a heavy chain variable domain (VH) with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VH sequence selected from the group consisting of the VH sequences listed in Table 1; and/or b) a light chain variable domain (VL) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VL sequence selected from the group consisting of the VL sequences listed in Table 2.

In certain embodiments, an antibody of the present disclosure is a humanized monoclonal antibody, or antigen-binding fragment or derivative thereof, wherein the antibody or antigen-binding fragment or derivative thereof comprises a) a heavy chain CDR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a heavy chain CDR sequence selected from the group consisting of the sequences listed in Table 3; and/or b) a light chain CDR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to alight chain CDR sequence selected from the group consisting of the sequences listed in Table 4.

In certain embodiments, an antibody of the present disclosure is a humanized monoclonal antibody, or antigen-binding fragment or derivative thereof, wherein the antibody or antigen-binding fragment or derivative thereof comprises a) a heavy chain FR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a heavy chain FR sequence selected from the group consisting of the sequences listed in Table 5; and/or b) a light chain FR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a light chain FR sequence selected from the group consisting of the sequences listed in Table 5.

In certain embodiments, an antibody of the present disclosure is a humanized monoclonal antibody, or antigen-binding fragment or derivative thereof, wherein the antibody or antigen-binding fragment or derivative thereof comprises a) a combination of a heavy chain CDR1, CDR2, and CDR3 as set forth in Table 3, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a combination of a light chain CDR1, CDR2, and CDR3 as set forth in Table 4, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In certain embodiments, an antibody of the present disclosure is a humanized monoclonal antibody, or antigen-binding fragment or derivative thereof, wherein the antibody or antigen-binding fragment or derivative thereof comprises: a) a VH sequence as set forth in Table 1, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a VL sequence as set forth in Table 2, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises a) a VH sequence selected from the group consisting of the VH sequences listed in Table 1; and/or b) a VL sequence selected from the group consisting of the VL sequences listed in Table 2.

In some embodiments, an antibody of the present disclosure is a humanized monoclonal antibody, or antigen-binding fragment or derivative thereof, wherein the antibody or antigen-binding fragment or derivative thereof comprises a) a VH sequence selected from the group consisting of SEQ ID NOs: 7, 12, 14, 17, and 32; and/or b) a VL sequence selected from the group consisting of SEQ ID NOs: 34, 65, 74, and 75.

In some embodiments, an antibody of the present disclosure is a humanized monoclonal antibody, or antigen-binding fragment or derivative thereof, wherein the antibody or antigen-binding fragment or derivative thereof comprises: six CDRs having the amino acid sequence set forth in SEQ ID NOs: 79, 80, 81, 101, 102, and 103, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises: six CDRs having the amino acid sequence set forth in SEQ ID NOs: 1-6, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises: six CDRs having the amino acid sequence set forth in SEQ ID NOs: 1-6, 79-133 and 152, or a variant sequence thereof which differs by only one or two amino acids, or which has at least or about 30%, 35%, 40%, 45%, 50%, 51%, 52%, 53%, 54%, 55%, 56%, %57% 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and d) a combination of a heavy chain CDR1, CDR2, and CDR3 as set forth in Table 3, or a variant sequence thereof which differs by only one or two amino acids, or which has at least or about 85% sequence identity thereto, and/or a combination of a light chain CDR1, CDR2, and CDR3 as set forth in Table 4, or a variant sequence thereof which differs by only one or two amino acids, or which has at least or about 85% sequence identity thereto.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 79 or 82;
(2) CDR-H2 having the amino acid sequence set forth in SEQ ID No: 80, 92, 94, or 95;
(3) CDR-H3 having the amino acid sequence set forth in SEQ ID No: 81;
(4) CDR-L1 having the amino acid sequence set forth in SEQ ID No: 101, 128, or 132;
(5) CDR-L2 having the amino acid sequence set forth in SEQ ID No: 102 or 105; and (6) CDR-L3 having the amino acid sequence set forth in SEQ ID No: 103, 106 or 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 79;
(2) CDR-H2 having the amino acid sequence set forth in SEQ ID No: 80, 92, 94, or 95; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 80, 92, 94, or 95; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 79;
(2) CDR-H2 having the amino acid sequence set forth in SEQ ID No: 80; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 80; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 92; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 94; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 95; and
(3) CDR-H3 having the amino acid sequence set forth in SEQ ID No: 81.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 101;
(5) CDR-L2 having the amino acid sequence set forth in SEQ ID No: 102 or 105; and
(6) CDR-L3 having the amino acid sequence set forth in SEQ ID No: 103, 106 or 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 128;
(5) CDR-L2 having the amino acid sequence set forth in SEQ ID No: 102 or 105; and
(6) CDR-L3 having the amino acid sequence set forth in SEQ ID No: 103, 106 or 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 132;
(5) CDR-L2 having the amino acid sequence set forth in SEQ ID No: 102 or 105; and
(6) CDR-L3 having the amino acid sequence set forth in SEQ ID No: 103, 106 or 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 101;
(5) CDR-L2 having the amino acid sequence set forth in SEQ ID No: 102; and
(6) CDR-L3 having the amino acid sequence set forth in SEQ ID No: 103.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 128;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 79;
(2) CDR-H2 having the amino acid sequence set forth in SEQ ID No: 80;
(3) CDR-H3 having the amino acid sequence set forth in SEQ ID No: 81;
(4) CDR-L1 having the amino acid sequence set forth in SEQ ID No: 101;
(5) CDR-L2 having the amino acid sequence set forth in SEQ ID No: 102; and
(6) CDR-L3 having the amino acid sequence set forth in SEQ ID No: 103.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 80;
(3) CDR-H3 having the amino acid sequence set forth in SEQ ID No: 81;
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 92;
(3) CDR-H3 having the amino acid sequence set forth in SEQ ID No: 81;
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 94;
(3) CDR-H3 having the amino acid sequence set forth in SEQ ID No: 81;
(4) CDR-L1 having the amino acid sequence set forth in SEQ ID No: 128;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 94;
(3) CDR-H3 having the amino acid sequence set forth in SEQ ID No: 81;
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 117.

In some embodiments, an antibody of the present disclosure is a humanized monoclonal antibody, or antigen-binding fragment or derivative thereof, wherein the antibody or antigen-binding fragment or derivative thereof comprises or consists of the following CDRs:
(1) CDR-H1 having the amino acid sequence set forth in SEQ ID No: 82;
(2) CDR-H2 having the amino acid sequence set forth in SEQ ID No: 95;
(3) CDR-H3 having the amino acid sequence set forth in SEQ ID No: 81;
(4) CDR-L1 having the amino acid sequence set forth in SEQ ID No: 132;
(5) CDR-L2 having the amino acid sequence set forth in SEQ ID No: 105; and
(6) CDR-L3 having the amino acid sequence set forth in SEQ ID No: 106.

In some embodiments, an antibody of the present disclosure is a humanized monoclonal antibody, or antigen-binding fragment or derivative thereof, wherein the antibody or antigen-binding fragment or derivative thereof comprises or consists of (a) a VH comprising or consisting of the amino acid sequence selected from: SEQ ID NOs: 7, 12, 14, 17 and 32, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%8, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence selected from SEQ ID NOs: 34, 65, 74, and 75, or a variant sequence thereof which differs by only one or two amino acids, or which has at least or about 30%, 35%, 40%, 45%, 50%, 51%, 52%5, 53% 54% 55%, 56%5, 57%, 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence selected from: SEQ ID NOs: 12, 14, 17 and 32, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence selected from SEQ ID NOs: 65, 74, and 75, or a variant sequence thereof which differs by only one or two amino acids, or which has at least or about 30%, 35%, 40%, 45%, 50%, 51%, 52%5, 53% 54% 55%, 56%5, 57%, 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence SEQ ID NOs: 12, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence SEQ ID NOs: 65, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence SEQ ID NOs: 14, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence SEQ ID NOs: 65, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence SEQ ID NOs: 17, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence SEQ ID NOs: 74, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence SEQ ID NOs: 17, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence SEQ ID NOs: 75, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody of the present disclosure is a humanized monoclonal antibody, or antigen-binding fragment or derivative thereof, wherein the antibody or antigen-binding fragment or derivative thereof comprises or consists of: (a) a VH comprising or consisting of the amino acid sequence SEQ ID NOs: 32, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or (b) a VL comprising or consisting of the amino acid sequence SEQ ID NOs: 65, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some of the embodiments described hereinabove and hereinbelow, the heavy chain and/or light chain framework regions comprise or consist of the following framework regions:
(1) FR1 having the amino acid sequence set forth in SEQ ID NO: 163 and/or 144;
(2) FR2 having the amino acid sequence set forth in SEQ ID NO: 164 and/or 145;
(3) FR3 having the amino acid sequence set forth in SEQ ID NO: 165 and/or 146; and
(4) FR4 having the amino acid sequence set forth in SEQ ID NO: 166 and/or 147.

In some of the embodiments described hereinabove and hereinbelow, the heavy chain framework regions comprise or consist of the following framework regions:
(1) FR1 having the amino acid sequence set forth in SEQ ID NO: 163;
(2) FR2 having the amino acid sequence set forth in SEQ ID NO: 164;
(3) FR3 having the amino acid sequence set forth in SEQ ID NO: 165; and
(4) FR4 having the amino acid sequence set forth in SEQ ID NO: 166.

In some of the embodiments described hereinabove and hereinbelow, the light chain framework regions comprise or consist of the following framework regions:
(1) FR1 having the amino acid sequence set forth in SEQ ID NO: 144;
(2) FR2 having the amino acid sequence set forth in SEQ ID NO: 145;
(3) FR3 having the amino acid sequence set forth in SEQ ID NO: 146; and
(4) FR4 having the amino acid sequence set forth in SEQ ID NO: 147.

In some of the embodiments described hereinabove and hereinbelow, an antibody or antigen-binding fragment or derivative thereof comprises heavy chain framework regions (HFR) and light chain framework regions (LFR); wherein said HFR comprise the following framework regions:
(1) FR1 having the amino acid sequence set forth in SEQ ID NO: 163;
(2) FR2 having the amino acid sequence set forth in SEQ ID NO: 164;
(3) FR3 having the amino acid sequence set forth in SEQ ID NO: 165;
(4) FR4 having the amino acid sequence set forth in SEQ ID NO: 166;
   and wherein said LFR comprise the following framework regions:
(1) FR1 having the amino acid sequence set forth in SEQ ID NO: 144;
(2) FR2 having the amino acid sequence set forth in SEQ ID NO: 145;
(3) FR3 having the amino acid sequence set forth in SEQ ID NO: 146; and
(4) FR4 having the amino acid sequence set forth in SEQ ID NO: 147.

In some of the embodiments described hereinabove and hereinbelow, an antibody or antigen-binding fragment or derivative thereof comprises heavy chain framework regions (HFR) and light chain framework regions (LFR); wherein said HFR comprise the following framework regions:
(1) FR1 having the amino acid sequence set forth in SEQ ID NO: 163;
(2) FR2 having the amino acid sequence set forth in SEQ ID NO: 164;
(3) FR3 having the amino acid sequence set forth in SEQ ID NO: 165;
(4) FR4 having the amino acid sequence set forth in SEQ ID NO: 166;
   and wherein said LFR comprise the following framework regions:
(1) FR1 having the amino acid sequence set forth in SEQ ID NO: 144;
(2) FR2 having the amino acid sequence set forth in SEQ ID NO: 145;
(3) FR3 having the amino acid sequence set forth in SEQ ID NO: 146; and
(4) FR4 having the amino acid sequence set forth in SEQ ID NO: 147;
   the antibody or antigen-binding fragment or derivative thereof being further characterized in that it comprises one or more, preferably all, of the following CDRs:
(1) CDR-H1 having the amino acid sequence set forth in SEQ ID No: 82;
(2) CDR-H2 having the amino acid sequence set forth in SEQ ID No: 92 or 94;
(3) CDR-H3 having the amino acid sequence set forth in SEQ ID No: 81;
(4) CDR-L1 having the amino acid sequence set forth in SEQ ID No: 128 or 132;
(5) CDR-L2 having the amino acid sequence set forth in SEQ ID No: 105; and
(6) CDR-L3 having the amino acid sequence set forth in SEQ ID No: 106 or 117.

In some of the embodiments described hereinabove and hereinbelow, an antibody or antigen-binding fragment or derivative thereof comprises at least the following CDRs:
(1) CDR-H3 having the amino acid sequence set forth in SEQ ID No: 81;
(2) CDR-L3 having the amino acid sequence set forth in SEQ ID No: 106 or 117.

In some of the embodiments described hereinabove and hereinbelow, an antibody or antigen-binding fragment or derivative thereof does not comprise at least one heavy chain framework region (HFR) selected from the group consisting of: SEQ ID NO: 134, SEQ ID NO: 135, SEQ ID NO: 136 and SEQ ID NO: 137. In some of the embodiments described hereinabove and hereinbelow, an antibody or antigen-binding fragment or derivative thereof does not comprise at least two, three or four of heavy chain framework region (HFR) selected from the group consisting of: SEQ ID NO: 134, SEQ ID NO: 135, SEQ ID NO: 136 and SEQ ID NO: 137.

In some of the embodiments described hereinabove and hereinbelow, an antibody or antigen-binding fragment or derivative thereof does not comprise at least one light chain framework region (LFR) selected from the group consisting of: SEQ ID NO: 178, SEQ ID NO: 179, SEQ ID NO: 180 and SEQ ID NO: 181. In some of the embodiments described hereinabove and hereinbelow, an antibody or antigen-binding fragment or derivative thereof does not comprise at least two, three or four of light chain framework region (HFR) selected from the group consisting of: SEQ ID NO: 178, SEQ ID NO: 179, SEQ ID NO: 180 and SEQ ID NO: 181.

Numerous embodiments are further provided that can be applied to any aspect of the present technology described herein. For example, in some embodiments, an antibody for use in the methods of the present disclosure is a monoclonal antibody, or antigen-binding fragment or derivative thereof, which is chimeric, humanized, composite, murine, or human. In certain embodiments, the antibody is a humanized monoclonal antibody. In some embodiments, a monoclonal antibody, or antigen-binding fragment or derivative thereof, comprises an immunoglobulin heavy chain constant domain selected from the group consisting of human IgG1, IgG2, IgG2A, IgG2B, IgG3, IgG4, IgA, IgM, IgD, and IgE constant domains. In some embodiments, a monoclonal antibody, or antigen-binding fragment or derivative thereof, is detectably labeled, comprises an effector domain, comprises an Fc domain, and/or is selected from the group consisting of Fv, F(ab′)2, Fab′, dsFv, scFv, sc(Fv)2 fragments, diabodies, bivalent, multivalent, and bifunctional engineered constructs. In some embodiments, a monoclonal antibody, or antigen-binding fragment or derivative thereof, is obtainable from hybridoma.

In certain aspects, an antibody for use in the methods of the present disclosure is a conjugate comprising the monoclonal antibody, or antigen-binding fragment or derivative thereof, described herein is provided. In some such embodiments, the conjugate comprises an additional moiety such as, for example and without limitation, a second therapeutic agent, a detectable moiety (e.g., a fluorophore, an enzyme, a radioisotope, etc.), and the like.

In certain aspects, an antibody for use in the compositions and methods of the present disclosure comprises an immunoglobulin heavy and/or light chain selected from the group consisting of immunoglobulin heavy and light chain sequences listed in Tables 1 and 2, respectively.

### Sequence Identity/Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. An antigen-binding and/or function-conservative variant also includes a polypeptide which has at least 60% amino acid identity as determined by BLAST or FASTA algorithms, preferably at least 75%, more preferably at least 80%, still more preferably at least 85%, still preferably at least 90%, and even more preferably at least 95%, and which has the same or substantially similar properties, binding specificities and/or functions as the native or parent protein to which it is compared.

The percent identity between two sequences is a function of the number of identical positions shared by the sequences (i.e., % identity=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 80%, at least or about 85%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Exemplary sequences are shown in Tables 1-6. Sequences are written, from left to right, from the N-terminal to the C-terminal. Residues which are changed compared to A01 are shown in bold and double underlined.

TABLE 1
Exemplary VH sequences of anti-GluN1 antibodies in accordance with certain
embodiments.

| | | SEQ ID |
|---|---|---|
| NAME | VH SEQUENCE (N→C) | NO: |
| A01 | QVQLVQSGAEVKKPGASVKVSCKASGYSFTGYYMFWVRQAPGQGLEWIGEINPSTGGA | 7 |
| | TYNLKFKARVTLTVDKSTSTAYMELSSLRSEDTAVYYCARLDALDYWGQGTLVTVSS | |
| B16 | QVQLVQSGAEVKKPGASVKVSCKASGYSFTGYYM [symbol] WVRQAPGQGLEWIGEINPSTGGA | 8 |
| | [symbol] YNLKFKARVTLTVDKSTSTAYMELSSLRSEDTAVYYCARL [symbol] LDYWGQGTLVTVSS | |
| B01 | QVQLVQSGAEVKKPGASVKVSCKASGYSFTGYYM [symbol] WVRQAPGQGLEWIGEINPSTGGA | 9 |
| | TYNLKFKARVTLTVDKSTSTAYMELSSLRSEDTAVYYCARL [symbol] LDYWGQGTLVTVSS | |
| B02 | QVQLVQSGAEVKKPGASVKVSCKASGYSFTGYYM [symbol] WVRQAPGQGLEWIGEINPSTGGA | 10 |
| | [symbol] YNLKFKARVTLTVDKSTSTAYMELSSLRSEDTAVYYCARL [symbol] LDYWGQGTLVTVSS | |
| B03 | QVQLVQSGAEVKKPGASVKVSCKASGYSFTGYYM [symbol] WVRQAPGQGLEWIGEINPSTG [symbol] A | 11 |
| | [symbol] YNLKFKARVTLTVDKSTSTAYMELSSLRSEDTAVYYCARL [symbol] LDYWGQGTLVTVSS | |
| B04 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| B05 | QVQLVQSGAEVKKPGASVKVSCKASGYSFTGYYM [symbol] WVRQAPGQGLEWIGEINPSTGGA | 13 |
| | [symbol] YNLKFKARVTLTVDKSTSTAYMELSSLRSEDTAVYYCARL [symbol] LDYWGQGTLVTVSS | |
| B06 | QVQLVQSGAEVKKPGASVKVSCKASGYSFTGYYM [symbol] WVRQAPGQGLEWIGEINPSTGGA | 14 |
| | [symbol] YNLKFKARVTLTVDKSTSTAYMELSSLRSEDTAVYYCARLDALDYWGQGTLVTVSS | |
| B07 | QVQLVQSGAEVKKPGASVKVSCKASGYSFTGYYM [symbol] WVRQAPGQGLEWIGEINP [symbol] TGGA | 15 |
| | TYNLKFKARVTLTVDKSTSTAYMELSSLRSEDTAVYYCARLDALDYWGQGTLVTVSS | |
| B08 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| B09 | QVQLVQSGAEVKKPGASVKVSCKASGYSFTGYYM [symbol] WVRQAPGQGLEWIGEINP [symbol] TGGA | 16 |
| | [symbol] YNLKFKARVTLTVDKSTSTAYMELSSLRSEDTAVYYCARLDALDYWGQGTLVTVSS | |
| B10 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| B11 | QVQLVQSGAEVKKPGASVKVSCKASGYSFTGYYM [symbol] WVRQAPGQGLEWIGEINPSTG [symbol] A | 18 |
| | TYNLKFKARVTLTVDKSTSTAYMELSSLRSEDTAVYYCARL [symbol] LDYWGQGTLVTVSS | |
| B12 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| B13 | QVQLVQSGAEVKKPGASVKVSCKASGYSFTGYYM [symbol] WVRQAPGQGLEWIGEINP [symbol] TGGA | 19 |
| | [symbol] YNLKFKARVTLTVDKSTSTAYMELSSLRSEDTAVYYCARL [symbol] LDYWGQGTLVTVSS | |
| B14 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| B15 | QVQLVQSGAEVKKPGASVKVSCKASGYSFTGYYM [symbol] WVRQAPGQGLEWIGEINPS [symbol] GGA | 20 |
| | [symbol] YNLKFKARVTLTVDKSTSTAYMELSSLRSEDTAVYYCARLDALDYWGQGTLVTVSS | |
| B17 | QVQLVQSGAEVKKPGASVKVSCKASGYSFTGYYM [symbol] WVRQAPGQGLEWIGEINP [symbol] TGGA | 21 |
| | TYNLKFKARVTLTVDKSTSTAYMELSSLRSEDTAVYYCARL [symbol] LDYWGQGTLVTVSS | |
| B18 | QVQLVQSGAEVKKPGASVKVSCKASGYSFTGYYM [symbol] WVRQAPGQGLEWIGEI [symbol] PSTGGA | 22 |
| | TYNLKFKARVTLTVDKSTSTAYMELSSLRSEDTAVYYCARL [symbol] LDYWGQGTLVTVSS | |
| B19 | QVQLVQSGAEVKKPGASVKVSCKASGYSFTGYYM [symbol] WVRQAPGQGLEWIGEINP [symbol] TGGA | 21 |
| | TYNLKFKARVTLTVDKSTSTAYMELSSLRSEDTAVYYCARL [symbol] LDYWGQGTLVTVSS | |
| B20 | QVQLVQSGAEVKKPGASVKVSCKASGYSFTGYYM [symbol] WVRQAPGQGLEWIGEI [symbol] PSTGGA | 22 |
| | TYNLKFKARVTLTVDKSTSTAYMELSSLRSEDTAVYYCARL [symbol] LDYWGQGTLVTVSS | |
| B21 | QVQLVQSGAEVKKPGASVKVSCKASGYSFTGYYM [symbol] WVRQAPGQGLEWIGEINP [symbol] TGGA | 21 |
| | TYNLKFKARVTLTVDKSTSTAYMELSSLRSEDTAVYYCARL [symbol] LDYWGQGTLVTVSS | |
| B22 | QVQLVQSGAEVKKPGASVKVSCKASGYSFTGYYM [symbol] WVRQAPGQGLEWIGEI [symbol] PSTGGA | 22 |
| | TYNLKFKARVTLTVDKSTSTAYMELSSLRSEDTAVYYCARL [symbol] LDYWGQGTLVTVSS | |
| A02 | [symbol] VQL [symbol] QSG [symbol] KPGASVK [symbol] SCKASGYSFTGYYMFWV [symbol] Q [symbol] P [symbol] LEWIGEINPSTGGA | 23 |
| | TYNLKFKA [symbol] TLTVDKS [symbol] TAYM [symbol] L [symbol] SL [symbol] SED [symbol] AVYYCARLDALDYWGQGT [symbol] VTVSS | |
| A03 | [symbol] VQL [symbol] QSG [symbol] KPGASVK [symbol] SCKASGYSFTGYYM [symbol] WV [symbol] Q [symbol] P [symbol] LEWIGEINPSTGGA | 24 |
| | TYNLKFKA [symbol] TLTVDKS [symbol] TAYMQL [symbol] SL [symbol] SED [symbol] AVYYCARLDALDYWGQGT [symbol] VTVSS | |
| C01 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C02 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C03 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C04 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C05 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C06 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C07 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C08 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C09 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C10 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C11 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C12 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C13 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C14 | QVQLVQSGAEVKKPGASVKVSCKASGYSFTGYYM [symbol] WVRQAPGQGLEWIGEINPSTGGA | 25 |
| | [symbol] YNLKFKARVTLTVDKSTSTAYMELSSLRSEDTAVYYCARL [symbol] LDYWGQGTLVTVSS | |
| C15 | QVQLVQSGAEVKKPGASVKVSCKASGYSFTGYYM [symbol] WVRQAPGQGLEWIGEINPSTGGA | 25 |
| | [symbol] YNLKFKARVTLTVDKSTSTAYMELSSLRSEDTAVYYCARL [symbol] LDYWGQGTLVTVSS | |
| C16 | QVQLVQSGAEVKKPGASVKVSCKASGYSFTGYYM [symbol] WVRQAPGQGLEWIGEINPSTGGA | 26 |
| | [symbol] YNLKFKARVTLTVDKSTSTAYMELSSLRSEDTAVYYCARL [symbol] ALDYWGQGTLVTVSS | |
| C17 | QVQLVQSGAEVKKPGASVKVSCKASGYSFTGYYM [symbol] WVRQAPGQGLEWIGEINPSTGGA | 26 |
| | [symbol] YNLKFKARVTLTVDKSTSTAYMELSSLRSEDTAVYYCARL [symbol] ALDYWGQGTLVTVSS | |
| C18 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| C19 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| C20 | QVQLVQSGAEVKKPGASVKVSCKASGYSFTGYYM [symbol] WVRQAPGQGLEWIGEINPSTGGA | 28 |
| | [symbol] YNLKFKARVTLTVDKSTSTAYMELSSLRSEDTAVYYCARLD [symbol] LDYWGQGTLVTVSS | |
| C21 | QVQLVQSGAEVKKPGASVKVSCKASGYSFTGYYM [symbol] WVRQAPGQGLEWIGEINPSTGGA | 28 |
| | [symbol] YNLKFKARVTLTVDKSTSTAYMELSSLRSEDTAVYYCARLD [symbol] LDYWGQGTLVTVSS | |
| C22 | QVQLVQSGAEVKKPGASVKVSCKASGYSFTGYYM [symbol] WVRQAPGQGLEWIGEINPSTGGA | 29 |
| | TYNLKFKARVTLTVDKSTSTAYMELSSLRSEDTAVYYCARL [symbol] ALDYWGQGTLVTVSS | |
| C23 | QVQLVQSGAEVKKPGASVKVSCKASGYSFTGYYM [symbol] WVRQAPGQGLEWIGEINPSTGGA | 29 |
| | TYNLKFKARVTLTVDKSTSTAYMELSSLRSEDTAVYYCARL [symbol] ALDYWGQGTLVTVSS | |
| C24 | QVQLVQSGAEVKKPGASVKVSCKASGYSFTGYYM [symbol] WVRQAPGQGLEWIGEINPSTGGA | 30 |
| | TYNLKFKARVTLTVDKSTSTAYMELSSLRSEDTAVYYCARLD [symbol] LDYWGQGTLVTVSS | |
| C25 | QVQLVQSGAEVKKPGASVKVSCKASGYSFTGYYM [symbol] WVRQAPGQGLEWIGEINPSTGGA | 30 |
| | TYNLKFKARVTLTVDKSTSTAYMELSSLRSEDTAVYYCARLD [symbol] LDYWGQGTLVTVSS | |
| C26 | QVQLVQSGAEVKKPGASVKVSCKASGYSFTGYYM [symbol] WVRQAPGQGLEWIGEINPSTGGA | 31 |
| | TYNLKFKARVTLTVDKSTSTAYMELSSLRSEDTAVYYCARL [symbol] LDYWGQGTLVTVSS | |
| C27 | QVQLVQSGAEVKKPGASVKVSCKASGYSFTGYYM [symbol] WVRQAPGQGLEWIGEINPSTGGA | 31 |
| | TYNLKFKARVTLTVDKSTSTAYMELSSLRSEDTAVYYCARL [symbol] LDYWGQGTLVTVSS | |
| C28 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| C29 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D01 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| D02 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| D03 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| D04 | QVQLVQSGAEVKKPGASVKVSCKASGYSFTGYYM [symbol] WVRQAPGQGLEWIGEINPSTGGA | 27 |
| | [symbol] YNLKFKARVTLTVDKSTSTAYMELSSLRSEDTAVYYCARLDALDYWGQGTLVTVSS | |
| D05 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D06 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D07 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D08 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D09 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D10 | QVQLVQSGAEVKKPGASVKVSCKASGYSFTGYYM [symbol] WVRQAPGQGLEWIGEINPSTGGA | 14 |
| | [symbol] YNLKFKARVTLTVDKSTSTAYMELSSLRSEDTAVYYCARLDALDYWGQGTLVTVSS | |
| D11 | QVQLVQSGAEVKKPGASVKVSCKASGYSFTGYYM [symbol] WVRQAPGQGLEWIGEINPSTGGA | 14 |
| | [symbol] YNLKFKARVTLTVDKSTSTAYMELSSLRSEDTAVYYCARLDALDYWGQGTLVTVSS | |
| D12 | QVQLVQSGAEVKKPGASVKVSCKASGYSFTGYYM [symbol] WVRQAPGQGLEWIGEINPSTGGA | 12 |
| | TYNLKFKARVTLTVDKSTSTAYMELSSLRSEDTAVYYCARLDALDYWGQGTLVTVSS | |
| D13 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| D14 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| D15 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| D16 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| D17 | QVQLVQSGAEVKKPGASVKVSCKASGYSFTGYYM [symbol] WVRQAPGQGLEWIGEI [symbol] PSTGGA | 32 |
| | [symbol] YNLKFKARVTLTVDKSTSTAYMELSSLRSEDTAVYYCARLDALDYWGQGTLVTVSS | |
| D18 | QVQLVQSGAEVKKPGASVKVSCKASGYSFTGYYM [symbol] WVRQAPGQGLEWIGEI [symbol] PSTGGA | 33 |
| | [symbol] YNLKFKARVTLTVDKSTSTAYMELSSLRSEDTAVYYCARLDALDYWGQGTLVTVSS | |
| D19 | QVQLVQSGAEVKKPGASVKVSCKASGYSFTGYYM [symbol] WVRQAPGQGLEWIGEI [symbol] PSTGGA | 32 |
| | [symbol] YNLKFKARVTLTVDKSTSTAYMELSSLRSEDTAVYYCARLDALDYWGQGTLVTVSS | |
| D20 | QVQLVQSGAEVKKPGASVKVSCKASGYSFTGYYM [symbol] WVRQAPGQGLEWIGEI [symbol] PSTGGA | 33 |
| | [symbol] YNLKFKARVTLTVDKSTSTAYMELSSLRSEDTAVYYCARLDALDYWGQGTLVTVSS | |
| D21 | QVQLVQSGAEVKKPGASVKVSCKASGYSFTGYYM [symbol] WVRQAPGQGLEWIGEI [symbol] PSTGGA | 32 |
| | [symbol] YNLKFKARVTLTVDKSTSTAYMELSSLRSEDTAVYYCARLDALDYWGQGTLVTVSS | |
| D22 | QVQLVQSGAEVKKPGASVKVSCKASGYSFTGYYM [symbol] WVRQAPGQGLEWIGEI [symbol] PSTGGA | 33 |
| | [symbol] YNLKFKARVTLTVDKSTSTAYMELSSLRSEDTAVYYCARLDALDYWGQGTLVTVSS | |
| D23 | QVQLVQSGAEVKKPGASVKVSCKASGYSFTGYYM [symbol] WVRQAPGQGLEWIGEI [symbol] PSTGGA | 32 |
| | [symbol] YNLKFKARVTLTVDKSTSTAYMELSSLRSEDTAVYYCARLDALDYWGQGTLVTVSS | |
| D24 | QVQLVQSGAEVKKPGASVKVSCKASGYSFTGYYM [symbol] WVRQAPGQGLEWIGEI [symbol] PSTGGA | 33 |
| | [symbol] YNLKFKARVTLTVDKSTSTAYMELSSLRSEDTAVYYCARLDALDYWGQGTLVTVSS | |
| D25 | QVQLVQSGAEVKKPGASVKVSCKASGYSFTGYYM [symbol] WVRQAPGQGLEWIGEI [symbol] PSTGGA | 17 |
| | TYNLKFKARVTLTVDKSTSTAYMELSSLRSEDTAVYYCARLDALDYWGQGTLVTVSS | |
| D26 | QVQLVQSGAEVKKPGASVKVSCKASGYSFTGYYM [symbol] WVRQAPGQGLEWIGEINPSTGGA | 14 |
| | [symbol] YNLKFKARVTLTVDKSTSTAYMELSSLRSEDTAVYYCARLDALDYWGQGTLVTVSS | |

TABLE 2
Exemplary VL sequences of anti-GluN1 antibodies in accordance with certain
embodiments.

| | | SEQ ID |
|---|---|---|
| NAME | VL SEQUENCE (N→C) | NO: |
| A01 | DVVMTQSPLSLPVTLGQPASISCRSSQSLVHSNGNTYLHWYQQRPGQSPRLLIYRVSN | 34 |
| | RFSGVPDRFSGSRSGTDFTLKISRVEAEDVGVYFCSQSTHVPFTFGQGTKLEIK | |
| B16 | DVVMTQSPLSLPVTLGQPASISCRSSQSLVHSNG [symbol] TYLHWYQQRPGQSPRLLIY [symbol] VSN | 35 |
| | RFSGVPDRFSGSRSGTDFTLKISRVEAEDVGVYFCSQSTH [symbol] PFTFGQGTKLEIK | |
| B01 | DVVMTQSPLSLPVTLGQPASISCRSSQSLV [symbol] SNGNTYLHWYQQRPGQSPRLLIY [symbol] VSN | 36 |
| | RFSGVPDRFSGSRSGTDFTLKISRVEAEDVGVYFCSQSTHVPFTFGQGTKLEIK | |
| B02 | DVVMTQSPLSLPVTLGQPASISCRSSQSLV [symbol] SNGNTYLHWYQQRPGQSPRLLIY [symbol] VSN | 37 |
| | RFSGVPDRFSGSRSGTDFTLKISRVEAEDVGVYFCSQSTHVP [symbol] TFGQGTKLEIK | |
| B03 | DVVMTQSPLSLPVTLGQPASISCRSSQSLVHSNGNTYLHWYQQRPGQSPRLLIY [symbol] VSN | 38 |
| | RFSGVPDRFSGSRSGTDFTLKISRVEAEDVGVYFCSQSTH [symbol] PFTFGQGTKLEIK | |
| B04 | DVVMTQSPLSLPVTLGQPASISCRSSQSLVH [symbol] NG [symbol] TYLHWYQQRPGQSPRLLIY [symbol] VSN | 39 |
| | RFSGVPDRFSGSRSGTDFTLKISRVEAEDVGVYFCSQSTH [symbol] PFTFGQGTKLEIK | |
| B05 | DVVMTQSPLSLPVTLGQPASISCRSSQSLVH [symbol] NGNTYLHWYQQRPGQSPRLLIY [symbol] VSN | 40 |
| | R [symbol] SGVPDRFSGSRSGTDFTLKISRVEAEDVGVYFCSQSTHVPFTFGQGTKLEIK | |
| B06 | DVVMTQSPLSLPVTLGQPASISCRSSQSLVHSNG [symbol] TYLHWYQQRPGQSPRLLIY [symbol] VSN | 41 |
| | RFSGVPDRFSGSRSGTDFTLKISRVEAEDVGVYFCSQSTH [symbol] PFTFGQGTKLEIK | |
| B07 | DVVMTQSPLSLPVTLGQPASISCRSSQSLVHSNG [symbol] TYLHWYQQRPGQSPRLLIY [symbol] VSN | 35 |
| | RFSGVPDRFSGSRSGTDFTLKISRVEAEDVGVYFCSQSTH [symbol] PFTFGQGTKLEIK | |
| B08 | DVVMTQSPLSLPVTLGQPASISCRSSQSLVHSNGNTYLHWYQQRPGQSPRLLIY [symbol] VSN | 42 |
| | RFSGVPDRFSGSRSGTDFTLKISRVEAEDVGVYFCSQST [symbol] P [symbol] TFGQGTKLEIK | |
| B09 | DVVMTQSPLSLPVTLGQPASISCRSSQSLV [symbol] SNGNTYLHWYQQRPGQSPRLLIY [symbol] VSN | 43 |
| | RFSGVPDRFSGSRSGTDFTLKISRVEAEDVGVYFCSQSTH [symbol] PFTFGQGTKLEIK | |
| B10 | DVVMTQSPLSLPVTLGQPASISCRSSQSLVHSNG [symbol] TYLHWYQQRPGQSPRLLIY [symbol] VSN | 44 |
| | RFSGVPDRFSGSRSGTDFTLKISRVEAEDVGVYFCSQSTH [symbol] P [symbol] TFGQGTKLEIK | |
| B11 | DVVMTQSPLSLPVTLGQPASISCRSSQSLVHSNGNTYLHWYQQRPGQSPRLLIYRVSN | 45 |
| | RFSGVPDRFSGSRSGTDFTLKISRVEAEDVGVYFCSQSTH [symbol] P [symbol] TFGQGTKLEIK | |
| B12 | DVVMTQSPLSLPVTLGQPASISCRSSQSLVHSNGNTYLHWYQQRPGQSPRLLIY [symbol] VSN | 46 |
| | RFSGVPDRFSGSRSGTDFTLKISRVEAEDVGVYFCSQSTH [symbol] PFTFGQGTKLEIK | |
| B13 | DVVMTQSPLSLPVTLGQPASISCRSSQSLV [symbol] S [symbol] G [symbol] TYLHWYQQRPGQSPRLLIYRVSN | 47 |
| | RFSGVPDRFSGSRSGTDFTLKISRVEAEDVGVYFCSQSTHVPFTFGQGTKLEIK | |
| B14 | DVVMTQSPLSLPVTLGQPASISCRSSQSLVH [symbol] NGNTYLHWYQQRPGQSPRLLIY [symbol] VSN | 48 |
| | RFSGVPDRFSGSRSGTDFTLKISRVEAEDVGVYFCSQST [symbol] PFTFGQGTKLEIK | |
| B15 | DVVMTQSPLSLPVTLGQPASISCRSSQSLVH [symbol] GNTYLHWYQQRPGQSPRLLIY [symbol] VSN | 49 |
| | RFSGVPDRFSGSRSGTDFTLKISRVEAEDVGVYFCSQSTH [symbol] PFTFGQGTKLEIK | |
| B17 | DVVMTQSPLSLPVTLGQPASISCRSSQSLVHS [symbol] G [symbol] TYLHWYQQRPGQSPRLLIYRVSN | 50 |
| | RFSGVPDRFSGSRSGTDFTLKISRVEAEDVGVYFCSQSTHVPFTFGQGTKLEIK | |
| B18 | DVVMTQSPLSLPVTLGQPASISCRSSQSLVHS [symbol] G [symbol] TYLHWYQQRPGQSPRLLIYRVSN | 50 |
| | RFSGVPDRFSGSRSGTDFTLKISRVEAEDVGVYFCSQSTHVPFTFGQGTKLEIK | |
| B19 | DVVMTQSPLSLPVTLGQPASISCRSSQSLVHS [symbol] G [symbol] TYLHWYQQRPGQSPRLLIYRVSN | 51 |
| | [symbol] FSGVPDRFSGSRSGTDFTLKISRVEAEDVGVYFCSQSTH [symbol] PFTFGQGTKLEIK | |
| B20 | DVVMTQSPLSLPVTLGQPASISCRSSQSLVHS [symbol] G [symbol] TYLHWYQQRPGQSPRLLIYRVSN | 51 |
| | [symbol] FSGVPDRFSGSRSGTDFTLKISRVEAEDVGVYFCSQSTH [symbol] PFTFGQGTKLEIK | |
| B21 | DVVMTQSPLSLPVTLGQPASISCRSSQSLV [symbol] S [symbol] G [symbol] TYLHWYQQRPGQSPRLLIYRVSN | 52 |
| | [symbol] FSGVPDRFSGSRSGTDFTLKISRVEAEDVGVYFCSQSTH [symbol] PFTFGQGTKLEIK | |
| B22 | DVVMTQSPLSLPVTLGQPASISCRSSQSLV [symbol] S [symbol] G [symbol] TYLHWYQQRPGQSPRLLIYRVSN | 52 |
| | [symbol] FSGVPDRFSGSRSGTDFTLKISRVEAEDVGVYFCSQSTH [symbol] PFTFGQGTKLEIK | |
| A02 | DVVMTQ [symbol] PLSLPV [symbol] LG [symbol] ASISCRSSQSLVHSNGNTYLHWY [symbol] Q [symbol] PGQSP [symbol] LLIYRVSN | 53 |
| | RFSGVPDRFSGSRSGTDFTLKISRVEAED [symbol] GVYFCSQSTHVPFTFG [symbol] GTKLEIK | |
| A03 | DVVMTQ [symbol] PLSLPV [symbol] LG [symbol] ASISCRSSQSLVHSNGNTYLHWY [symbol] Q [symbol] PGQSP [symbol] LLIYRVSN | 53 |
| | RFSGVPDRFSGSRSGTDFTLKISRVEAED [symbol] GVYFCSQSTHVPFTFG [symbol] GTKLEIK | |
| C01 | DVVMTQSPLSLPVTLGQPASISCRSSQSLVHSNGNTYLHWYQQRPGQSPRLLIYRVSN | 34 |
| | RFSGVPDRFSGSRSGTDFTLKISRVEAEDVGVYFCSQSTHVPFTFGQGTKLEIK | |
| C02 | DVVMTQSPLSLPVTLGQPASISCRSSQSLV [symbol] S [symbol] G [symbol] TYLHWYQQRPGQSPRLLIY [symbol] VSN | 54 |
| | RFSGVPDRFSGSRSGTDFTLKISRVEAEDVGVYFCSQSTH [symbol] PFTFGQGTKLEIK | |
| C03 | DVVMTQSPLSLPVTLGQPASISCRSSQSLV [symbol] S [symbol] G [symbol] TYLHWYQQRPGQSPRLLIY [symbol] VSN | 55 |
| | RFSGVPDRFSGSRSGTDFTLKISRVEAEDVGVYFCSQSTH [symbol] PFTFGQGTKLEIK | |
| C04 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C05 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C06 | DVVMTQSPLSLPVTLGQPASISCRSSQSLVHSNGNTYLHWYQQRPGQSPRLLIY [symbol] VSN | 46 |
| | RFSGVPDRFSGSRSGTDFTLKISRVEAEDVGVYFCSQSTH [symbol] PFTFGQGTKLEIK | |
| C07 | DVVMTQSPLSLPVTLGQPASISCRSSQSLVHS [symbol] G [symbol] TYLHWYQQRPGQSPRLLIY [symbol] VSN | 58 |
| | RFSGVPDRFSGSRSGTDFTLKISRVEAEDVGVYFCSQSTH [symbol] PFTFGQGTKLEIK | |
| C08 | DVVMTQSPLSLPVTLGQPASISCRSSQSLVHS [symbol] G [symbol] TYLHWYQQRPGQSPRLLIY [symbol] VSN | 59 |
| | RFSGVPDRFSGSRSGTDFTLKISRVEAEDVGVYFCSQSTH [symbol] PFTFGQGTKLEIK | |
| C09 | DVVMTQSPLSLPVTLGQPASISCRSSQSLVHS [symbol] G [symbol] TYLHWYQQRPGQSPRLLIY [symbol] VSN | 60 |
| | RFSGVPDRFSGSRSGTDFTLKISRVEAEDVGVYFCSQSTH [symbol] PFTFGQGTKLEIK | |
| C10 | DVVMTQSPLSLPVTLGQPASISCRSSQSLVHS [symbol] G [symbol] TYLHWYQQRPGQSPRLLIY [symbol] VSN | 61 |
| | RFSGVPDRFSGSRSGTDFTLKISRVEAEDVGVYFCSQSTH [symbol] PFTFGQGTKLEIK | |
| C11 | DVVMTQSPLSLPVTLGQPASISCRSSQSLV [symbol] S [symbol] G [symbol] TYLHWYQQRPGQSPRLLIY [symbol] VSN | 62 |
| | RFSGVPDRFSGSRSGTDFTLKISRVEAEDVGVYFCSQSTH [symbol] PFTFGQGTKLEIK | |
| C12 | DVVMTQSPLSLPVTLGQPASISCRSSQSLV [symbol] SHG [symbol] TYLHWYQQRPGQSPRLLIY [symbol] VSN | 63 |
| | RFSGVPDRFSGSRSGTDFTLKISRVEAEDVGVYFCSQSTH [symbol] PFTFGQGTKLEIK | |
| C13 | DVVMTQSPLSLPVTLGQPASISCRSSQSLV [symbol] SKG [symbol] TYLHWYQQRPGQSPRLLIY [symbol] VSN | 64 |
| | RFSGVPDRFSGSRSGTDFTLKISRVEAEDVGVYFCSQSTH [symbol] PFTFGQGTKLEIK | |
| C14 | DVVMTQSPLSLPVTLGQPASISCRSSQSLV [symbol] SN [symbol] TYLHWYQQRPGQSPRLLIY [symbol] VSN | 56 |
| | RESGVPDRFSGSRSGTDFTLKISRVEAEDVGVYFCSQSTH [symbol] PFTFGQGTKLEIK | |
| C15 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C16 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C17 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C18 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C19 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C20 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C21 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C22 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C23 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C24 | DVVMTQSPLSLPVTLGQPASISCRSSQSLV [symbol] SN [symbol] TYLHWYQQRPGQSPRLLIY [symbol] VSN | 56 |
| | RFSGVPDRFSGSRSGTDFTLKISRVEAEDVGVYFCSQSTH [symbol] PFTFGQGTKLEIK | |
| C25 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C26 | DVVMTQSPLSLPVTLGQPASISCRSSQSLV [symbol] SN [symbol] TYLHWYQQRPGQSPRLLIYQVSN | 56 |
| | RFSGVPDRFSGSRSGTDFTLKISRVEAEDVGVYFCSQSTH [symbol] PFTFGQGTKLEIK | |
| C27 | DVVMTQSPLSLPVTLGQPASISCRSSQSLV [symbol] SN [symbol] TYLHWYQQRPGQSPRLLIY [symbol] VSN | 57 |
| | RFSGVPDRFSGSRSGTDFTLKISRVEAEDVGVYFCSQSTH [symbol] PFTFGQGTKLEIK | |
| C28 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| C29 | DVVMTQSPLSLPVTLGQPASISCRSSQSLVHSN [symbol] TYLHWYQQRPGQSPRLLIYYVSN | 66 |
| | RFSGVPDRFSGSRSGTDFTLKISRVEAEDVGVYFCSQSTH [symbol] PFTFGQGTKLEIK | |
| D01 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| D02 | DVVMTQSPLSLPVTLGQPASISCRSSQSLVHS [symbol] TYLHWYQQRPGQSPRLLIY [symbol] VSN | 67 |
| | RFSGVPDRFSGSRSGTDFTLKISRVEAEDVGVYFCSQSTH [symbol] PFTFGQGTKLEIK | |
| D03 | DVVMTQSPLSLPVTLGQPASISCRSSQSLVHSN [symbol] TYLHWYQQRPGQSPRLLIY [symbol] VSN | 68 |
| | RFSGVPDRFSGSRSGTDFTLKISRVEAEDVGVYFCSQSTA [symbol] PFTFGQGTKLEIK | |
| D04 | DVVMTQSPLSLPVTLGQPASISCRSSQSLVHS [symbol] TYLHWYQQRPGQSPRLLIY [symbol] VSN | 69 |
| | RFSGVPDRFSGSRSGTDFTLKISRVEAEDVGVYFCSQST [symbol] PFTFGQGTKLEIK | |
| D05 | DVVMTQSPLSLPVTLGQPASISCRSSQSLVHS [symbol] TYLHWYQQRPGQSPRLLIY [symbol] VSN | 67 |
| | RFSGVPDRFSGSRSGTDFTLKISRVEAEDVGVYFCSQSTH [symbol] PFTFGQGTKLEIK | |
| D06 | DVVMTQSPLSLPVTLGQPASISCRSSQSLVHSN [symbol] TYLHWYQQRPGQSPRLLIY [symbol] VSN | 68 |
| | RFSGVPDRFSGSRSGTDFTLKISRVEAEDVGVYFCSQSTA [symbol] PFTFGQGTKLEIK | |
| D07 | DVVMTQSPLSLPVTLGQPASISCRSSQSLVHS [symbol] TYLHWYQQRPGQSPRLLIY [symbol] VSN | 69 |
| | RFSGVPDRFSGSRSGTDFTLKISRVEAEDVGVYFCSQST [symbol] PFTFGQGTKLEIK | |
| D08 | DVVMTQSPLSLPVTLGQPASISCRSSQSLVHSN [symbol] TYLHWYQQRPGQSPRLLIY [symbol] VSN | 70 |
| | RFSGVPDRFSGSRSGTDFTLKISRVEAEDVGVYFCSQST [symbol] P [symbol] TFGQGTKLEIK | |
| D09 | DVVMTQSPLSLPVTLGQPASISCRSSQSLVHSN [symbol] TYLHWYQQRPGQSPRLLIY [symbol] VSN | 71 |
| | RFSGVPDRFSGSRSGTDFTLKISRVEAEDVGVYFCSQST [symbol] PFTFGQGTKLEIK | |
| D10 | DVVMTQSPLSLPVTLGQPASISCRSSQSLVHSN [symbol] TYLHWYQQRPGQSPRLLIY [symbol] VSN | 72 |
| | RFSGVPDRFSGSRSGTDFTLKISRVEAEDVGVYFCSQSTH [symbol] PFTFGQGTKLEIK | |
| D11 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| D12 | DVVMTQSPLSLPVTLGQPASISCRSSQSLVHSN [symbol] TYLHWYQQRPGQSPRLLIY [symbol] VSN | 72 |
| | RFSGVPDRFSGSRSGTDFTLKISRVEAEDVGVYFCSQSTHSPFTFGQGTKLEIK | |
| D13 | DVVMTQSPLSLPVTLGQPASISCRSSQSLVHSN [symbol] TYLHWYQQRPGQSPRLLIY [symbol] VSN | 73 |
| | RFSGVPDRFSGSRSGTDFTLKISRVEAEDVGVYFCSQSTH [symbol] P [symbol] TFGQGTKLEIK | |
| D14 | DVVMTQSPLSLPVTLGQPASISCRSSQSLVHSN [symbol] TYLHWYQQRPGQSPRLLIY [symbol] VSN | 74 |
| | RFSGVPDRFSGSRSGTDFTLKISRVEAEDVGVYFCSQSTH [symbol] PFTFGQGTKLEIK | |
| D15 | DVVMTQSPLSLPVTLGQPASISCRSSQSLVHSN [symbol] TYLHWYQQRPGQSPRLLIY [symbol] VSN | 75 |
| | RFSGVPDRFSGSRSGTDFTLKISRVEAEDVGVYFCSQSTH [symbol] P [symbol] TFGQGTKLEIK | |
| D16 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| D17 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| D18 | DVVMTQSPLSLPVTLGQPASISCRSSQSLVHSN [symbol] TYLHWYQQRPGQSPRLLIY [symbol] VSN | 65 |
| | RFSGVPDRFSGSRSGTDFTLKISRVEAEDVGVYFCSQSTH [symbol] PFTFGQGTKLEIK | |
| D19 | DVVMTQSPLSLPVTLGQPASISCRSSQSLVHSN [symbol] TYLHWYQQRPGQSPRLLIY [symbol] VSN | 72 |
| | RESGVPDRFSGSRSGTDFTLKISRVEAEDVGVYFCSQSTH [symbol] PFTFGQGTKLEIK | |
| D20 | DVVMTQSPLSLPVTLGQPASISCRSSQSLVHSN [symbol] TYLHWYQQRPGQSPRLLIY [symbol] VSN | 72 |
| | RFSGVPDRFSGSRSGTDFTLKISRVEAEDVGVYFCSQSTH [symbol] PFTFGQGTKLEIK | |
| D21 | DVVMTQSPLSLPVTLGQPASISCRSSQSLVHS [symbol] TYLHWYQQRPGQSPRLLIY [symbol] VSN | 76 |
| | RFSGVPDRFSGSRSGTDFTLKISRVEAEDVGVYFCSQSTH [symbol] P [symbol] TFGQGTKLEIK | |
| D22 | DVVMTQSPLSLPVTLGQPASISCRSSQSLVHS [symbol] TYLHWYQQRPGQSPRLLIY [symbol] VSN | 76 |
| | RFSGVPDRFSGSRSGTDFTLKISRVEAEDVGVYFCSQSTH [symbol] P [symbol] TFGQGTKLEIK | |
| D23 | DVVMTQSPLSLPVTLGQPASISCRSSQSLVHS [symbol] TYLHWYQQRPGQSPRLLIY [symbol] VSN | 77 |
| | RFSGVPDRFSGSRSGTDFTLKISRVEAEDVGVYFCSQSTH [symbol] P [symbol] TFGQGTKLEIK | |
| D24 | DVVMTQSPLSLPVTLGQPASISCRSSQSLVHS [symbol] TYLHWYQQRPGQSPRLLIY [symbol] VSN | 77 |
| | RFSGVPDRFSGSRSGTDFTLKISRVEAEDVGVYFCSQSTH [symbol] P [symbol] TFGQGTKLEIK | |
| D25 | DVVMTQSPLSLPVTLGQPASISCRSSQSLVHSN [symbol] TYLHWYQQRPGQSPRLLIY [symbol] VSN | 78 |
| | RFSGVPDRFSGSRSGTDFTLKISRVEAEDVGVYFCSQSTH [symbol] P [symbol] TFGQGTKLEIK | |
| D26 | DVVMTQSPLSLPVTLGQPASISCRSSQSLVHSN [symbol] TYLHWYQQRPGQSPRLLIY [symbol] VSN | 78 |
| | RFSGVPDRFSGSRSGTDFTLKISRVEAEDVGVYFCSQSTH [symbol] P [symbol] TFGQGTKLEIK | |

TABLE 3
Exemplary heavy chain Complementarity determining region (CDR)
sequences in accordance with certain embodiments.

| | | SEQ | | SEQ | | SEQ |
|---|---|---|---|---|---|---|
| | CDR- | ID | | ID | | ID |
| NAME | H1 | NO: | CDR-H2 | NO: | CDR-H3 | NO: |
| A01 | GYYMF | 79 | EINPSTGGATYNLKFKA | 80 | LDALDY | 81 |
| Consensus | GYYM [symbol] | 1 | EI [symbol] P [symbol] GX₁₈A [symbol] YNLKFKA | 2 | L [symbol] LDY | 3 |
| sequence | (X₁ is | | (X₂ is N, D or Y; X₃ is | | (X₅ is | |
| | F, N, | | S, G, D or Y; X₁₉ is T | | D, S, | |
| | S, G, | | or Q; X₁₈ is G, N or E; | | A, T, | |
| | T or | | and X₄ is T, D, H, E, | | or Q; X₆ | |
| | Y) | | S or Y) | | is A or | |
| | | | | | D | |
| B16 | GYYM [symbol] | 82 | EINPSTGGA [symbol] YNLKFKA | 83 | L [symbol] LDY | 84 |
| B01 | GYYM [symbol] | 85 | EINPSTGGATYNLKFKA | 80 | L [symbol] LDY | 84 |
| B02 | GYYM [symbol] | 86 | EINPSTGGA [symbol] YNLKFKA | 83 | L [symbol] LDY | 87 |
| B03 | GYYM [symbol] | 86 | EINPSTG [symbol] A [symbol] YNLKFKA | 160 | L [symbol] LDY | 88 |
| B04 | GYYM [symbol] | 82 | EINPSTGGATYNLKFKA | 80 | LDALDY | 81 |
| B05 | GYYM [symbol] | 89 | EINPSTGGA [symbol] YNLKFKA | 90 | L [symbol] LDY | 91 |
| B06 | GYYM [symbol] | 82 | EINPSTGGA [symbol] YNLKFKA | 92 | LDALDY | 81 |
| B07 | GYYM [symbol] | 82 | EINP [symbol] TGGATYNLKFKA | 161 | LDALDY | 81 |
| B08 | GYYM [symbol] | 82 | EINPSTGGATYNLKFKA | 80 | LDALDY | 81 |
| B09 | GYYM [symbol] | 82 | EINP [symbol] TGGA [symbol] YNLKFKA | 93 | LDALDY | 81 |
| B10 | GYYM [symbol] | 82 | EI [symbol] PSTGGATYNLKFKA | 94 | LDALDY | 81 |
| B11 | GYYM [symbol] | 86 | EINPSTG [symbol] ATYNLKFKA | 152 | L [symbol] LDY | 88 |
| B12 | GYYM [symbol] | 82 | EINPSTGGATYNLKFKA | 80 | LDALDY | 81 |
| B13 | GYYM [symbol] | 86 | EINP [symbol] TGGA [symbol] YNLKFKA | 162 | L [symbol] LDY | 84 |
| B14 | GYYM [symbol] | 82 | EINPSTGGATYNLKFKA | 80 | LDALDY | 81 |
| B15 | GYYM [symbol] | 99 | EINPS [symbol] GGA [symbol] YNLKFKA | 100 | LDALDY | 81 |
| B17 | GYYM [symbol] | 86 | EINP [symbol] TGGATYNLKFKA | 167 | L [symbol] LDY | 84 |
| B18 | GYYM [symbol] | 86 | EI [symbol] PSTGGATYNLKFKA | 168 | L [symbol] LDY | 84 |
| B19 | GYYM [symbol] | 86 | EINP [symbol] TGGATYNLKFKA | 167 | L [symbol] LDY | 84 |
| B20 | GYYM [symbol] | 86 | EI [symbol] PSTGGATYNLKFKA | 168 | L [symbol] LDY | 84 |
| B21 | GYYM [symbol] | 86 | EINP [symbol] TGGATYNLKFKA | 167 | L [symbol] LDY | 84 |
| B22 | GYYM [symbol] | 86 | EI [symbol] PSTGGATYNLKFKA | 168 | L [symbol] LDY | 84 |
| A02 | GYYMF | 79 | EINPSTGGATYNLKEKA | 80 | LDALDY | 81 |
| A03 | GYYM [symbol] | 82 | EINPSTGGATYNLKFKA | 80 | LDALDY | 81 |
| C01 | GYYM [symbol] | 82 | EINPSTGGATYNLKFKA | 80 | LDALDY | 81 |
| C02 | GYYM [symbol] | 82 | EINPSTGGATYNLKFKA | 80 | LDALDY | 81 |
| C03 | GYYM [symbol] | 82 | EINPSTGGATYNLKFKA | 80 | LDALDY | 81 |
| C04 | GYYM [symbol] | 82 | EINPSTGGATYNLKFKA | 80 | LDALDY | 81 |
| C05 | GYYM [symbol] | 82 | EINPSTGGATYNLKFKA | 80 | LDALDY | 81 |
| C06 | GYYM [symbol] | 82 | EINPSTGGATYNLKFKA | 80 | LDALDY | 81 |
| C07 | GYYM [symbol] | 82 | EINPSTGGATYNLKFKA | 80 | LDALDY | 81 |
| C08 | GYYM [symbol] | 82 | EINPSTGGATYNLKFKA | 80 | LDALDY | 81 |
| C09 | GYYM [symbol] | 82 | EINPSTGGATYNLKFKA | 80 | LDALDY | 81 |
| C10 | GYYM [symbol] | 82 | EINPSTGGATYNLKFKA | 80 | LDALDY | 81 |
| C11 | GYYM [symbol] | 82 | EINPSTGGATYNLKFKA | 80 | LDALDY | 81 |
| C12 | GYYM [symbol] | 82 | EINPSTGGATYNLKFKA | 80 | LDALDY | 81 |
| C13 | GYYM [symbol] | 82 | EINPSTGGATYNLKFKA | 80 | LDALDY | 81 |
| C14 | GYYM [symbol] | 82 | EINPSTGGA [symbol] YNLKFKA | 83 | L [symbol] LDY | 87 |
| C15 | GYYM [symbol] | 82 | EINPSTGGA [symbol] YNLKFKA | 83 | L [symbol] LDY | 87 |
| C16 | GYYM [symbol] | 82 | EINPSTGGA [symbol] YNLKFKA | 83 | L [symbol] ALDY | 98 |
| C17 | GYYM [symbol] | 82 | EINPSTGGA [symbol] YNLKFKA | 83 | L [symbol] ALDY | 98 |
| C18 | GYYM [symbol] | 82 | EINPSTGGA [symbol] YNLKFKA | 83 | LDALDY | 81 |
| C19 | GYYM [symbol] | 82 | EINPSTGGA [symbol] YNLKFKA | 83 | LDALDY | 81 |
| C20 | GYYM [symbol] | 82 | EINPSTGGA [symbol] YNLKFKA | 83 | LD [symbol] LDY | 97 |
| C21 | GYYM [symbol] | 82 | EINPSTGGA [symbol] YNLKFKA | 83 | LD [symbol] LDY | 97 |
| C22 | GYYM [symbol] | 82 | EINPSTGGATYNLKFKA | 80 | L [symbol] ALDY | 98 |
| C23 | GYYM [symbol] | 82 | EINPSTGGATYNLKFKA | 80 | L [symbol] ALDY | 98 |
| C24 | GYYM [symbol] | 82 | EINPSTGGATYNLKFKA | 80 | LD [symbol] LDY | 97 |
| C25 | GYYM [symbol] | 82 | EINPSTGGATYNLKFKA | 80 | LD [symbol] LDY | 97 |
| C26 | GYYM [symbol] | 82 | EINPSTGGATYNLKFKA | 80 | L [symbol] LDY | 87 |
| C27 | GYYM [symbol] | 82 | EINPSTGGATYNLKFKA | 80 | L [symbol] LDY | 87 |
| C28 | GYYM [symbol] | 82 | EINPSTGGATYNLKFKA | 80 | LDALDY | 81 |
| C29 | GYYM [symbol] | 82 | EINPSTGGATYNLKFKA | 80 | LDALDY | 81 |
| D01 | GYYM [symbol] | 82 | EINPSTGGA [symbol] YNLKFKA | 83 | LDALDY | 81 |
| D02 | GYYM [symbol] | 82 | EINPSTGGA [symbol] YNLKEKA | 83 | LDALDY | 81 |
| D03 | GYYM [symbol] | 82 | EINPSTGGA [symbol] YNLKFKA | 83 | LDALDY | 81 |
| D04 | GYYM [symbol] | 82 | EINPSTGGA [symbol] YNLKFKA | 83 | LDALDY | 81 |
| D05 | GYYM [symbol] | 82 | EINPSTGGATYNLKFKA | 80 | LDALDY | 81 |
| D06 | GYYM [symbol] | 82 | EINPSTGGATYNLKFKA | 80 | LDALDY | 81 |
| D07 | GYYM [symbol] | 82 | EINPSTGGATYNLKFKA | 80 | LDALDY | 81 |
| D08 | GYYM [symbol] | 82 | EINPSTGGATYNLKFKA | 80 | LDALDY | 81 |
| D09 | GYYM [symbol] | 82 | EINPSTGGATYNLKFKA | 80 | LDALDY | 81 |
| D10 | GYYM [symbol] | 82 | EINPSTGGA [symbol] YNLKFKA | 92 | LDALDY | 81 |
| D11 | GYYM [symbol] | 82 | EINPSTGGA [symbol] YNLKFKA | 92 | LDALDY | 81 |
| D12 | GYYM [symbol] | 82 | EINPSTGGATYNLKFKA | 80 | LDALDY | 81 |
| D13 | GYYM [symbol] | 82 | EI [symbol] PSTGGATYNLKFKA | 94 | LDALDY | 81 |
| D14 | GYYM [symbol] | 82 | EI [symbol] PSTGGATYNLKFKA | 94 | LDALDY | 81 |
| D15 | GYYM [symbol] | 82 | EI [symbol] PSTGGATYNLKFKA | 94 | LDALDY | 81 |
| D16 | GYYM [symbol] | 82 | EI [symbol] PSTGGATYNLKFKA | 94 | LDALDY | 81 |
| D17 | GYYM [symbol] | 82 | EI [symbol] PSTGGA [symbol] YNLKEKA | 95 | LDALDY | 81 |
| D18 | GYYM [symbol] | 82 | EI [symbol] PSTGGA [symbol] YNLKFKA | 96 | LDALDY | 81 |
| D19 | GYYM [symbol] | 82 | EI [symbol] PSTGGA [symbol] YNLKFKA | 95 | LDALDY | 81 |
| D20 | GYYM [symbol] | 82 | EI [symbol] PSTGGA [symbol] YNLKFKA | 96 | LDALDY | 81 |
| D21 | GYYM [symbol] | 82 | EI [symbol] PSTGGA [symbol] YNLKFKA | 95 | LDALDY | 81 |
| D22 | GYYM [symbol] | 82 | EI [symbol] PSTGGA [symbol] YNLKFKA | 96 | LDALDY | 81 |
| D23 | GYYM [symbol] | 82 | EI [symbol] DPSTGGA [symbol] YNLKFKA | 95 | LDALDY | 81 |
| D24 | GYYM [symbol] | 82 | EI [symbol] PSTGGA [symbol] YNLKFKA | 96 | LDALDY | 81 |
| D25 | GYYM [symbol] | 82 | EI [symbol] PSTGGATYNLKFKA | 94 | LDALDY | 81 |
| D26 | GYYM [symbol] | 82 | EINPSTGGA [symbol] YNLKFKA | 92 | LDALDY | 81 |

TABLE 4
Exemplary light chain Complementarity determining region (CDR) sequences
in accordance with certain embodiments.

| | | SEQ | | SEQ | | SEQ |
|---|---|---|---|---|---|---|
| | | ID | | ID | | ID |
| NAME | CDR-L1 | NO: | CDR-L2 | NO: | CDR-L3 | NO: |
| A01 | RSSQSLVHSNGNTYLH | 101 | RVSNRFS | 102 | SQSTHVPFT | 103 |
| Consensus | RSSQSLV [symbol] | 4 | [symbol] VSN [symbol] S | 5 | SQST [symbol] P [symbol] T | 6 |
| sequence | TYLH | | (X₁₁ is R, Q | | (X₁₃ is H, A | |
| | (X₇ is H, A, E or D; | | or Y; X₁₂ is | | or E; X₁₄ is | |
| | X₁₆ is S, E or Q; X₈ | | R, Q or Y; | | V, E, S, D or | |
| | is N, R, S, Q, H or | | X₁₇ is F or H) | | Y; X₁₅ is F, | |
| | K; X₉ is G or A; X₁₀ | | | | S, T or Y) | |
| | is N, H or R) | | | | | |
| B16 | RSSQSLVHSNG [symbol] TYLH | 104 | [symbol] VSNRFS | 105 | SQSTH [symbol] PFT | 106 |
| B01 | RSSQSLV [symbol] SNGNTYLH | 107 | [symbol] VSNRFS | 108 | SQSTHVPFT | 103 |
| B02 | RSSQSLV [symbol] SNGNTYLH | 107 | [symbol] VSNRFS | 108 | SQSTHVP [symbol] T | 109 |
| B03 | RSSQSLVHSNGNTYLH | 101 | [symbol] VSNRFS | 108 | SQSTH [symbol] PFT | 110 |
| B04 | RSSQSLVH [symbol] NG [symbol] TYLH | 111 | [symbol] VSNRFS | 105 | SQSTH [symbol] PFT | 112 |
| B05 | RSSQSLVH [symbol] NGNTYLH | 113 | [symbol] VSNR [symbol] S | 169 | SQSTHVPFT | 103 |
| B06 | RSSQSLVHSNG [symbol] TYLH | 104 | [symbol] VSNRFS | 105 | SQSTH [symbol] PFT | 110 |
| B07 | RSSQSLVHSNG [symbol] TYLH | 104 | [symbol] VSNRFS | 105 | SQSTH [symbol] PFT | 106 |
| B08 | RSSQSLVHSNGNTYLH | 101 | [symbol] VSNRFS | 105 | SQST [symbol] P [symbol] T | 114 |
| B09 | RSSQSLV [symbol] SNGNTYLH | 115 | [symbol] VSNRFS | 105 | SQSTH [symbol] PFT | 106 |
| B10 | RSSQSLVHSNG [symbol] TYLH | 116 | [symbol] VSNRFS | 105 | SQSTH [symbol] P [symbol] T | 117 |
| B11 | RSSQSLVHSNGNTYLH | 101 | RVSNRFS | 102 | SQSTH [symbol] P [symbol] T | 117 |
| B12 | RSSQSLVHSNGNTYLH | 101 | [symbol] VSNRFS | 105 | SQSTH [symbol] PFT | 106 |
| B13 | RSSQSLV [symbol] S [symbol] G [symbol] TYLH | 118 | RVSNRFS | 102 | SQSTHVPFT | 103 |
| B14 | RSSQSLVH [symbol] NGNTYLH | 119 | [symbol] VSNRFS | 105 | SQST [symbol] PFT | 170 |
| B15 | RSSQSLVH [symbol] GNTYLH | 120 | [symbol] VSNRFS | 105 | SQSTH [symbol] PFT | 171 |
| B17 | RSSQSLVHS [symbol] G [symbol] TYLH | 121 | RVSNRFS | 102 | SQSTHVPFT | 103 |
| B18 | RSSQSLVHS [symbol] G [symbol] TYLH | 121 | RVSNRFS | 102 | SQSTHVPFT | 103 |
| B19 | RSSQSLVHS [symbol] G [symbol] TYLH | 122 | RVSN [symbol] FS | 172 | SQSTH [symbol] PFT | 106 |
| B20 | RSSQSLVHS [symbol] G [symbol] TYLH | 122 | RVSN [symbol] FS | 172 | SQSTH [symbol] PFT | 106 |
| B21 | RSSQSLV [symbol] S [symbol] G [symbol] TYLH | 123 | RVSN [symbol] FS | 173 | SQSTH [symbol] PFT | 106 |
| B22 | RSSQSLV [symbol] S [symbol] G [symbol] TYLH | 123 | RVSN [symbol] FS | 173 | SQSTH [symbol] PFT | 106 |
| A02 | RSSQSLVHSNGNTYLH | 101 | RVSNRFS | 102 | SQSTHVPFT | 103 |
| A03 | RSSQSLVHSNGNTYLH | 101 | RVSNRFS | 102 | SQSTHVPFT | 103 |
| C01 | RSSQSLVHSNGNTYLH | 101 | RVSNRFS | 102 | SQSTHVPFT | 103 |
| C02 | RSSQSLV [symbol] S [symbol] G [symbol] TYLH | 124 | [symbol] VSNRFS | 105 | SQSTH [symbol] PFT | 106 |
| C03 | RSSQSLV [symbol] S [symbol] G [symbol] TYLH | 124 | [symbol] VSNRFS | 108 | SQSTH [symbol] PFT | 106 |
| C04 | RSSQSLV [symbol] SN [symbol] TYLH | 125 | [symbol] VSNRFS | 105 | SQSTH [symbol] PFT | 106 |
| C05 | RSSQSLV [symbol] SN [symbol] TYLH | 125 | [symbol] VSNRFS | 108 | SQSTH [symbol] PFT | 106 |
| C06 | RSSQSLVHSNGNTYLH | 101 | [symbol] VSNRFS | 105 | SQSTH [symbol] PFT | 106 |
| C07 | RSSQSLVHS [symbol] G [symbol] TYLH | 122 | [symbol] VSNRFS | 105 | SQSTH [symbol] PFT | 106 |
| C08 | RSSQSLVHS [symbol] G [symbol] TYLH | 126 | [symbol] VSNRFS | 105 | SQSTH [symbol] PFT | 106 |
| C09 | RSSQSLVHS [symbol] G [symbol] TYLH | 127 | [symbol] VSNRFS | 105 | SQSTH [symbol] PFT | 106 |
| C10 | RSSQSLVHS [symbol] G [symbol] TYLH | 128 | [symbol] VSNRFS | 105 | SQSTH [symbol] PFT | 106 |
| C11 | RSSQSLV [symbol] S [symbol] G [symbol] TYLH | 129 | [symbol] VSNRFS | 105 | SQSTH [symbol] PFT | 106 |
| C12 | RSSQSLV [symbol] S [symbol] G [symbol] TYLH | 130 | [symbol] VSNRFS | 105 | SQSTH [symbol] PFT | 106 |
| C13 | RSSQSLV [symbol] S [symbol] G [symbol] TYLH | 131 | [symbol] VSNRFS | 105 | SQSTH [symbol] PFT | 106 |
| C14 | RSSQSLV [symbol] SN [symbol] TYLH | 125 | [symbol] VSNRFS | 105 | SQSTH [symbol] PFT | 106 |
| C15 | RSSQSLV [symbol] SN [symbol] TYLH | 125 | [symbol] VSNRFS | 108 | SQSTH [symbol] PFT | 106 |
| C16 | RSSQSLV [symbol] SN [symbol] TYLH | 125 | [symbol] VSNRFS | 105 | SQSTH [symbol] PFT | 106 |
| C17 | RSSQSLV [symbol] SN [symbol] TYLH | 125 | [symbol] VSNRFS | 108 | SQSTH [symbol] PFT | 106 |
| C18 | RSSQSLV [symbol] SN [symbol] TYLH | 125 | [symbol] VSNRFS | 105 | SQSTH [symbol] PFT | 106 |
| C19 | RSSQSLV [symbol] SN [symbol] TYLH | 125 | [symbol] VSNRFS | 108 | SQSTH [symbol] PFT | 106 |
| C20 | RSSQSLV [symbol] SN [symbol] TYLH | 125 | [symbol] VSNRFS | 105 | SQSTH [symbol] PFT | 106 |
| C21 | RSSQSLV [symbol] SN [symbol] TYLH | 125 | [symbol] VSNRFS | 108 | SQSTH [symbol] PFT | 106 |
| C22 | RSSQSLV [symbol] SN [symbol] TYLH | 125 | [symbol] VSNRFS | 105 | SQSTH [symbol] PFT | 106 |
| C23 | RSSQSLV [symbol] SN [symbol] TYLH | 125 | [symbol] VSNRFS | 108 | SQSTH [symbol] PFT | 106 |
| C24 | RSSQSLV [symbol] SN [symbol] TYLH | 125 | [symbol] VSNRFS | 105 | SQSTH [symbol] PFT | 106 |
| C25 | RSSQSLV [symbol] SN [symbol] TYLH | 125 | [symbol] VSNRFS | 108 | SQSTH [symbol] PFT | 106 |
| C26 | RSSQSLV [symbol] SN [symbol] TYLH | 125 | [symbol] VSNRFS | 105 | SQSTH [symbol] PFT | 106 |
| C27 | RSSQSLV [symbol] SN [symbol] TYLH | 125 | [symbol] VSNRFS | 108 | SQSTH [symbol] PFT | 106 |
| C28 | RSSQSLVHSN [symbol] TYLH | 132 | [symbol] VSNRFS | 105 | SQSTH [symbol] PFT | 106 |
| C29 | RSSQSLVHSN [symbol] TYLH | 132 | [symbol] VSNRFS | 108 | SQSTH [symbol] PFT | 106 |
| D01 | RSSQSLVHSN [symbol] TYLH | 132 | [symbol] VSNRFS | 105 | SQSTH [symbol] PFT | 106 |
| D02 | RSSQSLVHS [symbol] TYLH | 133 | [symbol] VSNRFS | 105 | SQSTH [symbol] PFT | 106 |
| D03 | RSSQSLVHSN [symbol] TYLH | 132 | [symbol] VSNRFS | 105 | SQST [symbol] PFT | 174 |
| D04 | RSSQSLVHS [symbol] TYLH | 133 | [symbol] VSNRFS | 105 | SQST [symbol] PFT | 174 |
| D05 | RSSQSLVHS [symbol] TYLH | 133 | [symbol] VSNRFS | 105 | SQSTH [symbol] PFT | 106 |
| D06 | RSSQSLVHSN [symbol] TYLH | 132 | [symbol] VSNRFS | 105 | SQST [symbol] PFT | 174 |
| D07 | RSSQSLVHS [symbol] TYLH | 133 | [symbol] VSNRFS | 105 | SQST [symbol] PFT | 174 |
| D08 | RSSQSLVHSN [symbol] TYLH | 132 | [symbol] VSNRFS | 105 | SQST [symbol] P [symbol] T | 114 |
| D09 | RSSQSLVHSN [symbol] TYLH | 132 | [symbol] VSNRFS | 105 | SQST [symbol] PFT | 175 |
| D10 | RSSQSLVHSN [symbol] TYLH | 132 | [symbol] VSNRFS | 105 | SQSTH [symbol] PFT | 110 |
| D11 | RSSQSLVHSN [symbol] TYLH | 132 | [symbol] VSNRFS | 105 | SQSTH [symbol] PFT | 106 |
| D12 | RSSQSLVHSN [symbol] TYLH | 132 | [symbol] VSNRFS | 105 | SQSTH [symbol] PFT | 110 |
| D13 | RSSQSLVHSN [symbol] TYLH | 128 | [symbol] VSNRFS | 105 | SQSTH [symbol] P [symbol] T | 117 |
| D14 | RSSQSLVHSN [symbol] TYLH | 128 | [symbol] VSNRFS | 105 | SQSTH [symbol] PFT | 106 |
| D15 | RSSQSLVHSN [symbol] TYLH | 132 | [symbol] VSNRFS | 105 | SQSTH [symbol] P [symbol] T | 117 |
| D16 | RSSQSLVHSN [symbol] TYLH | 132 | [symbol] VSNRFS | 105 | SQSTH [symbol] PFT | 106 |
| D17 | RSSQSLVHSN [symbol] TYLH | 132 | [symbol] VSNRFS | 105 | SQSTH [symbol] PFT | 106 |
| D18 | RSSQSLVHSN [symbol] TYLH | 132 | [symbol] VSNRFS | 105 | SQSTH [symbol] PFT | 106 |
| D19 | RSSQSLVHSN [symbol] TYLH | 132 | [symbol] VSNRFS | 105 | SQSTH [symbol] PFT | 110 |
| D20 | RSSQSLVHSN [symbol] TYLH | 132 | [symbol] VSNRFS | 105 | SQSTH [symbol] PFT | 110 |
| D21 | RSSQSLVHS [symbol] TYLH | 133 | [symbol] VSNRFS | 105 | SQSTH [symbol] P [symbol] T | 117 |
| D22 | RSSQSLVHS [symbol] TYLH | 133 | [symbol] VSNRFS | 105 | SQSTH [symbol] P [symbol] T | 117 |
| D23 | RSSQSLVHS [symbol] TYLH | 133 | [symbol] VSNRFS | 105 | SQSTH [symbol] P [symbol] T | 177 |
| D24 | RSSQSLVHS [symbol] TYLH | 133 | [symbol] VSNRFS | 105 | SQSTH [symbol] P [symbol] T | 177 |
| D25 | RSSQSLVHSN [symbol] TYLH | 132 | [symbol] VSNRFS | 105 | SQSTH [symbol] P [symbol] T | 177 |
| D26 | RSSQSLVHSN [symbol] TYLH | 132 | [symbol] VSNRFS | 105 | SQSTH [symbol] P [symbol] T | 177 |

TABLE 5
Exemplary Heavy chain Framework region (FR) sequences in accordance with
certain embodiments.

| | | SEQ | | SEQ | | SEQ | | SEQ |
|---|---|---|---|---|---|---|---|---|
| | | ID | | ID | | ID | | ID |
| NAME | H-FR1 | NO: | H-FR2 | NO: | H-FR3 | NO: | H-FR4 | NO: |
| A02/ | [symbol] VQL [symbol] QSG [symbol] | 134 | WV [symbol] Q [symbol] P | 135 | [symbol] TLTVDKS [symbol] TAY | 136 | WGQGT | 137 |
| A03 | KPGASVK [symbol] SCKA | | [symbol] LEW | | M [symbol] L [symbol] SL [symbol] SED [symbol] AVY | | [symbol] VTVS | |
| | SGYSFT | | IG | | YCAR | | S | |
| 15A4- | QVQLVQSGAEVK | 163 | WVRQAP | 164 | RVTLTVDKSTSTAY | 165 | WGQGT | 166 |
| 146- | KPGASVKVSCKA | | GQGLEW | | MELSSLRSEDTAVY | | LVTVS | |
| VHA | SGYSFT | | IG | | YCAR | | S | |
| 15A4- | QVQLVQSGAEVK | 163 | WVRQAP | 164 | RVTLTVDKS [symbol] STAY | 138 | WGQGT | 166 |
| 12- | KPGASVKVSCKA | | GQGLEW | | MELS [symbol] LRS [symbol] DT [symbol] VY | | LVTVS | |
| VHA | SGYSFT | | IG | | YCAR | | S | |
| 15A4- | QVL [symbol] Q [symbol] GA [symbol] | 139 | W [symbol] RQ [symbol] P | 140 | RVTL [symbol] VDKS [symbol] | 141 | WGQGT | 166 |
| 4341- | KP [symbol] C [symbol] A | | G [symbol] GLEW | | [symbol] LSS [symbol] DTAVY | | LVTVS | |
| VHA | [symbol] GYSFT | | IG | | YCAR | | S | |
| 15A4- | QVQL [symbol] Q [symbol] GA [symbol] | 139 | W [symbol] RQ [symbol] P | 140 | RVTL [symbol] VDKS [symbol] | 141 | WGQGT | 166 |
| 4341- | KP [symbol] C [symbol] A | | G [symbol] GLEW | | [symbol] LSS [symbol] DTAVY | | LVTVS | |
| VHB | [symbol] GYSFT | | IG | | YCAR | | S | |
| 15A4- | QVQL [symbol] Q [symbol] GA [symbol] | 139 | W [symbol] RQ [symbol] P | 140 | RVTL [symbol] VDKS [symbol] A [symbol] | 142 | WGQGT | 166 |
| 4341- | KP [symbol] C [symbol] A | | G [symbol] GLEW | | [symbol] LSS [symbol] DTAVY | | LVTVS | |
| VHC | [symbol] GYSFT | | IG | | YCAR | | S | |
| 15A4- | QVQL [symbol] SG [symbol] | 143 | W [symbol] RQ [symbol] P | 140 | RVTL [symbol] VDKS [symbol] | 141 | WGQGT | 166 |
| 4349- | KP [symbol] C [symbol] A | | G [symbol] GLEW | | [symbol] LSS [symbol] DTAVY | | LVTVS | |
| VHA | [symbol] GYSFT | | IG | | YCAR | | S | |
| 15A4- | QVQL [symbol] SG [symbol] | 143 | W [symbol] RQ [symbol] P | 140 | RVTL [symbol] VDKS [symbol] A [symbol] | 142 | WGQGT | 166 |
| 4349- | KP [symbol] C [symbol] A | | G [symbol] GLEW | | [symbol] LSS [symbol] DTAVY | | LVTVS | |
| VHB | [symbol] GYSFT | | IG | | YCAR | | S | |

TABLE 6
Exemplary Light chain Framework region (FR) sequences in
accordance with certain embodiments.

| | | SEQ | | SEQ | | SEQ | | SEQ |
|---|---|---|---|---|---|---|---|---|
| | | ID | | ID | | ID | | ID |
| NAME | L-FR1 | NO: | L-FR2 | NO: | L-FR3 | NO: | L-FR4 | NO: |
| A02/ | DVVMTQ [symbol] PLSLP | 178 | WY [symbol] Q [symbol] PG | 179 | GVPDRFSGSRSGTD | 180 | FG [symbol] GT | 181 |
| A03 | V [symbol] LG [symbol] ASISC | | QSP [symbol] LLI | | FTLKISRVEAED [symbol] G | | KLEIK | |
| | | | Y | | VYFC | | | |
| 15A4- | DVVMTQSPLSLP | 144 | WYQQRPG | 145 | GVPDRFSGSRSGTD | 146 | FGQGT | 147 |
| 230- | VTLGQPASISC | | QSPRLLI | | FTLKISRVEAEDVG | | KLEIK | |
| VLA | | | Y | | VYFC | | | |
| 15A4- | DVVMTQ [symbol] P [symbol] SLP | 148 | W [symbol] LQ [symbol] PG | 149 | GVPDRFSGSRSGTD | 150 | FGQGT | 147 |
| 218- | V [symbol] G [symbol] PASISC | | QSP [symbol] LLI | | FTLKIS [symbol] VEAEDVG | | KLEIK | |
| VLA | | | Y | | VYFC | | | |

### Nucleic Acids, Vectors, and Recombinant Host Cells

A further object of the disclosure relates to nucleic acid sequences encoding humanized monoclonal antibodies and antigen-binding fragments or derivatives thereof, immunoglobulins, and polypeptides, for use in the compositions and methods of the present disclosure.

For example, in certain embodiments, the present disclosure relates, in part, to a nucleic acid sequence encoding the VH domain or the VL domain of the antibodies or antigen-binding fragments or derivatives thereof for use in the methods of the present disclosure.

Typically, said nucleic acid is a DNA or RNA molecule, which may be included in any suitable vector, such as a plasmid, cosmid, episome, artificial chromosome, phage or a viral vector.

Exemplary DNA sequences encoding the VH and VL sequences shown in Table 1 and Table 2 are provided in the sequence listing (SEQ ID NOs: 184 to 355). The DNA sequences provided in SEQ ID NOs: 184 to 355 include a DNA sequence at the 5′-end that encodes for a 19aa signal peptide at the N-terminus of the protein sequence. Exemplary signal sequences are shown in Table 7. Proteins and polypeptides of the disclosure are generally expressed with a secretion signal sequence at the N-terminus which is cleaved off during expression and is not included in the purified and/or mature proteins and polypeptides.

Exemplary DNA sequences encoding the CH and CL sequences, without the signal peptide, are shown in Table 7bis and in the sequence listing (SEQ ID NOs: 356, 357). Exemplary full-length light and heavy chains, including the constant heavy chain (CH) polypeptide sequence and the constant light chain (CL) polypeptide sequence are further shown in Table 7bis and in the sequence listing (SEQ ID NOs: 358, 359, 360, 361).

TABLE 7
Exemplary signal sequence used for protein expression in accordance
with certain embodiments.

| DNA sequence encoding the signal peptide (5′→3′) | SEQ ID NO. |
|---|---|
| ATG GGC TGG TCC TGC ATC ATC CTG TTT CTG GTG GCT ACC GCT ACC | 158 |
| GGC GTG CAC TCT | |
| Signal peptide (N→C) | |
| MGWSCIILFLVATATGVHS | 176 |

TABLE 7_{bis}
Exemplary nucleic acid and polypeptide sequences used for protein
expression in accordance with certain embodiments.

| NAME | DNA sequence (5′→3′) | SEQ ID NO. |
|---|---|---|
| D11 kappa | GACGTCGTGATGACCCAGTCTCCTCTGTCCCTTCCCGTGACCCTGGGGCA | 356 |
| light chain | GCCTGCGTCCATCTCCTGTCGGAGCAGCCAGAGCCTTGTGCACTCCAACG | |
| coding | CCCACACTTACCTCCATTGGTACCAGCAGCGCCCAGGACAGTCGCCGAGG | |
| sequence | CTCCTGATCTACCAAGTGTCCAACCGGTTCAGCGGAGTGCCTGATAGATT | |
| (without | CAGCGGATCGAGAAGCGGAACGGACTTTACCCTGAAAATCAGCCGCGTGG | |
| signal | AAGCCGAGGACGTGGGCGTGTATTTCTGCTCACAATCCACCCACGAGCCC | |
| peptide) | TTCACATTCGGACAGGGCACCAAACTCGAGATCAAGCGCACCGTGGCAGC | |
| | TCCGTCAGTGTTCATTTTTCCGCCCTCCGACGAACAGTTGAAGTCCGGTA | |
| | CCGCCTCGGTGGTCTGTCTGCTGAACAATTTCTACCCGCGGGAAGCCAAA | |
| | GTGCAGTGGAAGGTCGATAACGCACTGCAATCAGGGAACAGCCAGGAATC | |
| | CGTGACTGAGCAGGACTCGAAGGATTCCACTTACTCCCTGTCCTCCACCC | |
| | TGACCCTGTCGAAGGCCGACTACGAGAAGCATAAGGTCTACGCCTGCGAA | |
| | GTGACCCACCAAGGACTGTCATCCCCCGTGACTAAGTCCTTCAACCGGGG | |
| | CGAATGCTGA | |
| D11 IgG1- | CAAGTGCAACTGGTGCAGAGCGGAGCAGAAGTGAAAAAGCCTGGAGCCTC | 357 |
| LALA heavy | CGTAAAAGTGTCCTGTAAAGCGTCCGGATACAGCTTCACCGGCTACTACA | |
| chain coding | TGAACTGGGTCCGCCAAGCGCCGGGCCAGGGCTTGGAATGGATCGGAGAG | |
| sequence | ATCAACCCGTCCACCGGTGGCGCCGAGTATAACCTCAAGTTCAAGGCCCG | |
| (without | CGTGACCTTGACTGTGGACAAGTCCACCTCCACCGCCTACATGGAACTGA | |
| signal | GCAGCCTGAGATCCGAGGATACAGCCGTGTACTATTGCGCTCGGCTTGAC | |
| peptide) | GCTCTGGACTACTGGGGACAGGGCACCCTCGTGACTGTGTCGAGCGCGTC | |
| | CACTAAGGGTCCGTCCGTGTTTCCGCTGGCCCCTAGCTCCAAGTCAACCT | |
| | CAGGAGGAACCGCCGCACTCGGTTGCCTCGTGAAGGACTACTTCCCCGAA | |
| | CCCGTCACCGTGTCCTGGAATAGCGGAGCGCTGACATCCGGAGTGCACAC | |
| | CTTTCCTGCGGTGCTTCAGTCATCCGGACTGTACTCCCTGTCGTCTGTGG | |
| | TCACTGTTCCGTCCTCCTCCCTGGGGACCCAGACTTACATTTGCAACGTG | |
| | AACCACAAGCCCAGCAACACCAAGGTCGACAAGAGAGTGGAGCCGAAGTC | |
| | CTGCGATAAGACCCATACTTGCCCTCCATGCCCGGCCCCCGAGGCCGCTG | |
| | GAGGTCCCTCCGTGTTCCTCTTCCCACCAAAGCCTAAGGACACCCTGATG | |
| | ATCTCGCGCACCCCGGAAGTCACTTGTGTGGTGGTGGATGTGTCACATGA | |
| | GGACCCAGAAGTCAAGTTCAATTGGTACGTCGACGGCGTCGAAGTGCACA | |
| | ACGCCAAGACTAAGCCCCGGGAGGAACAGTACAACTCCACCTACCGGGTG | |
| | GTGTCGGTGCTCACTGTGCTGCACCAGGATTGGCTGAATGGGAAGGAGTA | |
| | TAAGTGCAAAGTGTCAAACAAGGCCTTGCCCGCTCCTATTGAAAAGACGA | |
| | TCTCGAAGGCCAAGGGCCAGCCTAGGGAGCCCCAGGTCTACACTCTGCCG | |
| | CCTTCACGAGAAGAGATGACCAAGAACCAAGTGTCGCTGACCTGTCTCGT | |
| | GAAAGGGTTTTACCCCTCCGACATCGCCGTGGAATGGGAAAGCAACGGCC | |
| | AGCCGGAGAACAACTACAAGACGACCCCTCCCGTCCTGGACTCCGATGGG | |
| | AGCTTCTTCCTGTACTCTAAGCTGACCGTGGACAAGAGTCGGTGGCAGCA | |
| | AGGAAACGTGTTCAGCTGCTCGGTCATGCATGAGGCCCTGCACAACCACT | |
| | ACACTCAGAAGTCGCTCAGCCTTTCGCCGGGGAAATGA | |
| NAME | Polypeptide sequence (N→C) | |
| D11 kappa | DVVMTQSPLSLPVTLGQPASISCRSSQSLVHSNAHTYLHWYQQRPGQSP | 358 |
| light chain | RLLIYQVSNRFSGVPDRFSGSRSGTDFTLKISRVEAEDVGVYFCSQSTHE | |
| (without | PFTFGQGTKLEIKRTVAAPSVFIFPPSDEQLKSGTASVVCLLNNFYPREA | |
| signal | KVQWKVDNALQSGNSQESVTEQDSKDSTYSLSSTLTLSKADYEKHKVYAC | |
| peptide) | EVTHQGLSSPVTKSfNRGEC | |
| D11 IgG1- | QVQLVQSGAEVKKPGASVKVSCKASGYSFTGYYMNWVRQAPGQGLEWIGE | 359 |
| LALA heavy | INPSTGGAEYNLKFKARVTLTVDKSTSTAYMELSSLRSEDTAVYYCARLD | |
| chain | ALDYWGQGTLVTVSSASTKGPSVFPLAPSSKSTSGGTAALGCLVKDYFPE | |
| (without | PVTVSWNSGALTSGVHTFPAVLQSSGLYSLSSVVTVPSSSLGTQTYICNV | |
| signal | NHKPSNTKVDKRVEPKSCDKTHTCPPCPAPEAAGGPSVFLFPPKPKDTLM | |
| peptide) | ISRTPEVTCVVVDVSHEDPEVKFNWYVDGVEVHNAKTKPREEQYNSTYRV | |
| | VSVLTVLHQDWLNGKEYKCKVSNKALPAPIEKTISKAKGQPREPQVYTLP | |
| | PSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| D11 kappa | RTVAAPSVFIFPPSDEQLKSGTASVVCLLNNFYPREAKVQWKVDNALQSG | 360 |
| light chain | NSQESVTEQDSKDSTYSLSSTLTLSKADYEKHKVYACEVTHQGLSSPVTK | |
| constant | SFNRGEC | |
| region (CL) | | |
| D11 IgG1- | ASTKGPSVFPLAPSSKSTSGGTAALGCLVKDYFPEPVTVSWNSGALTSGV | 361 |
| LALA heavy | HTFPAVLQSSGLYSLSSVVTVPSSSLGTQTYICNVNHKPSNTKVDKRVEP | |
| chain | KSCDKTHTCPPCPAPEAAGGPSVFLFPPKPKDTLMISRTPEVTCVVVDVS | |
| constant | HEDPEVKFNWYVDGVEVHNAKTKPREEQYNSTYRVVSVLTVLHQDWLNGK | |
| region (CH) | EYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a vector comprising a nucleic acid may also be used in the methods of the present disclosure.

Such vectors may comprise regulatory elements, such as a promoter, enhancer, terminator and the like, to cause or direct expression of said polypeptide upon administration to a subject. Examples of promoters and enhancers used in the expression vector for animal cells include early promoter and enhancer of SV40, LTR promoter and enhancer of Moloney mouse leukemia virus, promoter and enhancer of immunoglobulin H chain and the like.

Any expression vector for animal cells can be used. Examples of suitable vectors include pAGE107, pAGE103, pHSG274, pKCR, pSG1 beta d2-4 and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The nucleic acids described herein may be used to produce a recombinant polypeptide in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective, rat YB2/3HL.P2.G11.16Ag.20 cell (ATCC CRL 1662, hereinafter referred to as “YB2/0 cell”), and the like. The YB2/0 cell is preferred in some embodiments, since ADCC activity of chimeric or humanized antibodies is enhanced when expressed in this cell.

In another aspect, the present disclosure provides isolated nucleic acids that hybridize under selective hybridization conditions to a polynucleotide disclosed herein for use in the methods of the present disclosure. Thus, the polynucleotides of this embodiment can be used for isolating, detecting, and/or quantifying nucleic acids comprising such polynucleotides. For example, polynucleotides can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides will encode at least a portion of an antibody encoded by the polynucleotides described herein. The polynucleotides may embrace nucleic acid sequences that can be employed for selective hybridization to a polynucleotide encoding an antibody for use in the compositions and methods of the present disclosure.

### Methods of Producing Antibodies

Antibodies and fragments thereof, immunoglobulins, and polypeptides for use in the methods of the present disclosure may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present disclosure can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques. For example, an antibody or a polypeptide for use in the methods of the present disclosure may be produced by a method comprising the steps of: (i) culturing a transformed host cell according to the disclosure under conditions suitable to allow expression of said antibody or polypeptide; and (ii) recovering the expressed antibody or polypeptide.

Antibodies and other polypeptides for use in the methods of the present disclosure may be suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class chimeric and humanized monoclonal antibodies, comprising both human and non-human portions, which can be made using standard recombinant DNA techniques. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J Immunol. 139:3521-3526; Sun et al. (1987) Proc. Natl. Acad. Sci. 84:214-218; Wood et al. (1985) Nature 314:446-449; Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. Humanized antibodies for use in the methods of the present disclosure can be produced by obtaining nucleic acid sequences encoding CDR domains, as previously described, constructing a humanized antibody expression vector by inserting them into an expression vector for animal cell having genes encoding (i) a CH identical to that of a human antibody and (ii) a CL identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type).

Antibodies can be humanized using a variety of techniques including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). Recombinant DNA technology and gene transfection techniques for preparation of such antibodies can be used (see European Patent Application EP 125023 and International Patent Application WO 96/02576).

In addition, antibody fragments can be produced using a variety of techniques. For example, Fab fragments of the present disclosure can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present disclosure can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present disclosure can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present disclosure can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

### Modification of Antibodies, Immunoglobulins, and Polypeptides

Amino acid sequence modification(s) of the antibodies described herein are contemplated in the present disclosure. For example, it may be desirable to improve the binding affinity and/or other biological properties of an antibody. It is known that when a humanized antibody is produced by simply grafting only CDRs in VH and VL of an antibody derived from a non-human animal in FRs of the VH and VL of a human antibody, the antigen binding activity may be reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody can reduce binding activity and can be corrected by replacing the amino acids with amino acid residues of the original antibody derived from a non-human animal.

Modifications and changes may be made in the structure of the antibodies for use in the methods of the present disclosure, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the disclosure, or corresponding DNA sequences which encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown above). Likewise, there is a known and definite correspondence between the nucleotide sequence of a particular nucleic acid and the amino acid sequence encoded by that nucleic acid, as defined by the genetic code (see genetic code chart above).

Another type of amino acid modification of the antibody of the disclosure may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation can be achieved using a variety of techniques, and enzymatic cleavage of carbohydrate moieties on antibodies can be achieved by the use of a variety of endo- and exo-glycosidases.

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present disclosure with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

### Pharmaceutical Compositions

In some embodiments of methods of the present disclosure, an antibody, or antigen-binding fragment or derivative thereof, is administered in the form of a pharmaceutical composition comprising the antibody, or antigen-binding fragment or derivative thereof, and a pharmaceutically acceptable carrier. As used herein, the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition for use in the methods of the present disclosure is formulated to be compatible with its intended route of administration. Examples of routes of administration include parenteral (e.g. intravenous, intradermal, subcutaneous, intraperitoneal, inhalation, transdermal, transmucosal) and enteral (e.g. oral, rectal) administration. Solutions or suspensions used for parenteral or enteral application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The pharmaceutical preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the compounds for use in the methods of the present disclosure are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by, and directly dependent on, the unique characteristics of the active compound, the particular therapeutic effect to be achieved, and the limitations inherent in the art of compounding such an active compound for the treatment of individuals.

### In Vitro Uses and Methods

In certain aspects, provided herein are in vitro uses of antibodies, fragments or derivatives thereof, or compositions, or nucleic acids or expression cassettes or vectors, or host cells, or pharmaceutical compositions.

In certain aspects, provided herein is an in vitro method for detecting glutamate ionotropic receptor NMDA type subunit 1 (GluN1) of a N-methyl-D-aspartate receptor (NMDAR) in a sample; the method comprising a step of bringing in contact a sample with antibodies, fragments or derivatives thereof according to the present disclosure, and detecting the binding of said antibodies, fragments or derivatives to the sample, thereby detecting the GluN1 subunit.

### Therapeutic and Diagnosis Methods

In certain aspects, provided herein are therapeutic and diagnosis methods comprising a step of administering antibodies, fragments or derivatives thereof, or compositions, or nucleic acids or expression cassettes or vectors, or host cells, or pharmaceutical compositions provided herein to a subject in an effective amount.

In some embodiments, antibodies, fragments or derivatives thereof, or compositions, or nucleic acids or expression cassettes or vectors, or host cells, or pharmaceutical compositions provided herein are for use as a medicament (for use in medicine).

In some embodiments, antibodies, fragments or derivatives thereof, or compositions, or nucleic acids or expression cassettes or vectors, or host cells, or pharmaceutical compositions provided herein are for use for the preparation of a medicament.

In some embodiments, antibodies, fragments or derivatives thereof, or compositions, or nucleic acids or expression cassettes or vectors, or host cells, or pharmaceutical compositions provided herein are for use in a method of diagnosis in vivo.

In certain aspects, provided herein are therapeutic methods for the prevention and treatment of a neurological, neurovascular or neurodegenerative disorder. For example, in certain aspects, provided herein is a method of treating a neurological, neurovascular or neurodegenerative disorder in a subject, the method comprising: administering to the subject a therapeutically effective amount of an antibody or antigen-binding fragment or derivative thereof, or pharmaceutical composition thereof, of the present disclosure, such that the neurological, neurovascular or neurodegenerative disorder is treated in the subject.

For example, in certain aspects, provided herein is a method of treating a neurological, neurovascular or neurodegenerative disorder in a subject, the method comprising: administering to the subject a therapeutically effective amount of nucleic acids or expression cassettes or vectors, or host cells, of the present disclosure, such that the neurological, neurovascular or neurodegenerative disorder is treated in the subject.

In some embodiments, antibodies, antigen-binding fragments or derivatives thereof, or compositions provided herein may be used alone or in combination with one or more additional therapeutic agents, e.g., other anti-NMDA receptor agents, other anti-neurological, neurovascular or neurodegenerative disorder therapies. Thus, antibodies, antigen-binding fragments or derivatives thereof and/or compositions described herein may be administered alone or in combination with one or more additional therapy. The latter can be administered before, after or simultaneously with the administration of the antibodies, antigen-binding fragments or derivatives thereof and/or compositions described herein.

In some embodiments, the antibody or antigen-binding fragment or derivative of the disclosure and the one or more additional therapeutic agents are present in a combined composition. In some embodiments, the antibody or antigen-binding fragment or derivative or composition of the disclosure and the one or more additional therapeutic agents are administered separately.

In some embodiments, a subject is in need of treatment by the methods provided herein and is selected for treatment based on this need. A subject in need of treatment is art-recognized, and includes subjects that have been identified as having a disease or condition (e.g., a neurological, neurovascular or neurodegenerative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

As used herein, “treating” or “treatment” of a disease or condition refers, in some embodiments, to ameliorating at least one disease or condition (i.e., arresting or reducing the development of a disease or condition or at least one of the clinical symptoms thereof). In certain embodiments “treating” or “treatment” refers to ameliorating at least one physical parameter, such as e.g. brain tumor size, growth, or migration. In certain embodiments, “treating” or “treatment” refers to inhibiting or improving a disease or condition, either physically (e.g., stabilization of a discernible symptom), physiologically (e.g., stabilization of a physical parameter), or both. In certain embodiments, “treating” or “treatment” refers to delaying the onset (or recurrence) of a disease or condition. The term “treating” or “treatment” may refer to any indicia of success in the treatment or amelioration of a disease or condition, including any objective or subjective parameter such as abatement; remission; diminishing of symptoms or making the disease or condition more tolerable to the subject; improving a subject's physical or mental well-being, such as reducing pain experienced by the patient; and, in some situations additionally improving at least one parameter of a disease or condition.

As used herein, “preventing” or “prevention” is intended to refer at least to the reduction of the likelihood of, or the risk of, or susceptibility to acquiring a disease or disorder (i.e., causing at least one of the clinical symptoms of the disease not to develop in a patient that may be exposed to or predisposed to or at risk of the disease but does not yet experience or display symptoms of the disease). The term “prevention” or “preventing” is also used to describe the administration of a compound or composition described herein to a subject who is at risk of (or susceptible to) such a disease or condition. Subjects amenable to treatment for prevention of a disease or condition include individuals at risk of the disease or condition but not showing symptoms, as well as patients presently showing symptoms. In some embodiments, “prevention” or “preventing” is used to describe the administration of a compound or composition described herein to a subject who has been diagnosed with or treated for a disease or condition and is at risk of recurrence of the disease or condition.

In some embodiments, treatment or prevention are within the context of the present disclosure if there is a measurable difference between the performances of subjects treated using the methods provided herein as compared to members of a placebo group, historical control, or between subsequent tests given to the same subject.

The term “therapeutically effective amount” as used herein means that amount or dose of a compound or composition, upon single or multiple dose administration to a subject, which provides the desired effect (e.g., the desired biological or medicinal response, e.g., to ameliorate, lessen or prevent a disease, disorder or condition) in the subject being treated. In some embodiments, an effective amount is an amount or dose of an antibody or antigen-binding fragment or derivative or composition that prevents or treats a neurological, neurovascular or neurodegenerative disorder in a subject, as described herein. The terms “effective amount” and “therapeutically effective amount” are used interchangeably herein.

It should be understood that the dosage or amount of a compound and/or composition used, alone or in combination with one or more active compounds to be administered, depends on the individual case and is, as is customary, to be adapted to the individual circumstances to achieve an optimum effect. Dosing and administration regimens are within the purview of the skilled artisan, and appropriate doses depend upon a number of factors within the knowledge of the ordinarily skilled physician, veterinarian, or researcher (e.g., see Wells et al. eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000)). For example, dosing and administration regimens depend on the nature and the severity of the disorder to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the therapeutic molecule(s).

Thus the dose(s) of a compound or composition will vary depending upon a variety of factors including, but not limited to: the activity, biological and pharmacokinetic properties and/or side effects of the compound being used; the age, body weight, general health, gender, and diet of the subject; the time of administration, the route of administration, the rate of excretion, and any drug combination, if applicable; the effect which the practitioner desires the compound to have upon the subject; and the properties of the compound being administered (e.g. bioavailability, stability, potency, toxicity, etc). Such appropriate doses may be determined as known in the art. When one or more of the compounds of the disclosure is to be administered to humans, a physician may for example, prescribe a relatively low dose at first, subsequently increasing the dose until an appropriate response is obtained.

There are no particular limitations on the dose of each of the compounds for use in compositions provided herein. Exemplary doses include milligram or microgram amounts of the compound per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligrams per kilogram, about 1 milligram per kilogram to about 10 milligrams per kilogram, or about 3 milligrams per kilogram to about 5 milligrams per kilogram). Additional exemplary doses include doses of about 5 to about 500 mg, about 25 to about 300 mg, about 25 to about 200 mg, about 50 to about 150 mg, or about 50, about 100, about 150 mg, about 200 mg or about 250 mg, and, for example, monthly, biweekly, weekly, every other day, daily or twice daily, or lower or higher amounts.

In some embodiments, the dose range for adult humans is generally from 0.005 mg to 10 g/day orally. Tablets or other forms of presentation provided in discrete units may conveniently contain an amount of a compound (e.g., of Formula I, or in Table 1 or 2) which is effective at such dosage or as a multiple of the same, for instance, units containing 5 mg to 500 mg, usually around 10 mg to 200 mg. A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg) of a compound described herein.

Administration of compounds and compositions provided herein can be carried out using known procedures, at dosages and for periods of time effective to achieve a desired purpose. Dosage regimens can be adjusted to provide the optimum therapeutic response. For example, several divided doses may be administered daily, or the dose may be proportionally reduced as indicated by the exigencies of the therapeutic situation. In some instances, the treatment regimen may vary over time, depending on selected protocols. For example, administration may be weekly during an initiation period, followed by administration every-other-month, or every-2-months, etc., during a chronic period. In some embodiments, a compound or composition is administered at an effective dosage sufficient to prevent or treat a neurological, neurovascular or neurodegenerative disorder in a subject. Further, a compound or composition may be administered using any suitable route or means, such as without limitation via oral, parenteral, intravenous, intraperitoneal, intramuscular, sublingual, topical, or nasal administration, via inhalation, or via such other routes as are known in the art.

### Kits

Compounds and compositions provided herein may be packaged as part of a kit, optionally including a container (e.g. packaging, a box, a vial, etc). The kit may be commercially used according to the methods described herein and may include instructions for use in such methods. Additional kit components may include acids, bases, buffering agents, inorganic salts, solvents, antioxidants, preservatives, or metal chelators. The additional kit components may be present as pure compositions, or as aqueous or organic solutions that incorporate one or more additional kit components. Any or all of the kit components optionally further comprise buffers.

### Methods of Use

The term “neurological disorder” is used herein to refer to diseases, disorders or conditions which directly or indirectly affect the normal functioning or anatomy of a subject's nervous system.

The term “neurovascular disorder” is used herein to refer to diseases, disorders or conditions which affect blood supply in the brain or spinal cord, e.g., narrowing, hardening, or abnormality of a blood vessel, blockage caused by a clot or embolism, or hemorrhage or bleeding from a ruptured blood vessel.

The term “neurodegenerative disorder” is used herein to refer to diseases, disorders or conditions in which cells of the central and/or peripheral nervous system are affected or lost. Examples of neurological, neurovascular or neurodegenerative disorders include, without limitation: multiple sclerosis (MS) and other demyelinating syndromes (such as neuromyelitis optica spectrum disorder, myelin oligodendrocyte glycoprotein antibody-associated disease, other anti-MOG spectrum disorders, anti-aquaporin 4 spectrum disorders, or demyelinating attack), thrombotic disorder, stroke, ischemic disorder, ischemic stroke, transient ischemic attack (TIA), intracerebral bleeding (including haemorrhagic stroke), traumatic brain injury (TBI), spinal cord injury (SCI), intracranial or intravertebral lesions including, but not limited to, contusion, penetration, shear, compression or laceration lesions of the spinal cord, whiplash, and shaken infant syndrome, brain trauma, impairments in learning, cognition and/or memory, migraine, epilepsy, temporal lobe epilepsy (TLE), amyotrophic lateral sclerosis (ALS), Huntington's disease, brain tumors, Parkinson's disease, schizophrenia, neuropathic pain, depression, anxiety, drug addiction, multiple system atrophy (MSA), Alzheimer's disease, age-related macular degeneration (AMD), aneurysm, brain oedema, CNS complication resulting from parasitic, bacterial, fungal or viral infection, meningitis, and encephalitis.

In certain embodiments, the neurological, neurovascular or neurodegenerative disorder is stroke. In some such embodiments, the stroke is ischemic stroke. In some such embodiments, the stroke is hemorrhagic stroke.

In certain embodiments, the neurological, neurovascular or neurodegenerative disorder is MS.

In certain embodiments, the neurological, neurovascular or neurodegenerative disorder is Parkinson's disease.

In some embodiments, the neurological and/or neurodegenerative disorder is an ischemic event, ischemia or an ischemic disorder, which can be defined as any local or regional state of hypoxia in cells or tissues, which is usually due to an inadequate blood supply (circulation), e.g., caused by a blockage or obstruction of a blood vessel in the affected area. The hypoxia can cause acute or chronic injury as in hypoxia and/or ischemia including, but not limited to, cerebrovascular insufficiency, cerebral ischemia or cerebral infarction (including cerebral ischemia or infarctions originating from embolic occlusion and thrombosis), retinal ischemia (diabetic or otherwise, such as by acute vascular occlusion, in particular damage of the optic nerve after acute vascular occlusion), glaucoma (in particular damage of the optic nerve in glaucoma), retinal degeneration, multiple sclerosis, ischemic optic neuropathy, reperfusion following acute cerebral ischemia, perinatal hypoxic-ischemic injury, or intracranial hemorrhage of any type (including, but not limited to, epidural, subdural, subarachnoid or intracerebral hemorrhage). Accordingly, the term “ischemic disorder” as used herein encompasses thrombotic disorders which include conditions associated with or resulting from thrombosis or a tendency towards thrombosis. These conditions include conditions associated with arterial or venous thrombosis that can be treated with a thrombolytic drug.

Accordingly, the present disclosure provides methods for the treatment and/or prevention of a neurological, neurovascular or neurodegenerative disorder in a subject, comprising administering to the subject a therapeutically effective amount of an anti-GluN1 antibody, or an antigen-binding fragment or derivative thereof of the disclosure, such that the neurological, neurovascular or neurodegenerative disorder is treated and/or prevented in the subject. In some embodiments of methods of the disclosure, the nucleic acid, expression cassette or vector, host cell, or pharmaceutical composition of the disclosure is administered to the subject. In some embodiments, the subject is a subject in need of such treatment. In some embodiments, the subject is a human.

The present disclosure also provides methods for the diagnosis, prognosis and/or monitoring of a disease using the anti-GluN1 antibody, or the antigen-binding fragment or derivative thereof, the nucleic acid, the expression cassette or vector, the host cell, or the pharmaceutical composition of the disclosure. In some embodiments, the anti-GluN1 antibody, or the antigen-binding fragment or derivative thereof is coupled to a labelling agent which is capable of producing a detectable signal. Non-limiting examples of labelling agents include radionuclides, fluorophores and enzymes.

In the methods of the present disclosure, therapeutic anti-GluN1 antibodies and antigen-binding fragments or derivatives thereof, and compositions thereof, can be used alone or can be used in combination with one or more additional treatment or agent for neurological, neurovascular or neurodegenerative disorders. The preceding treatment methods can be administered in conjunction with other forms of conventional therapy (e.g., standard-of-care treatments for neurological, neurovascular or neurodegenerative disease well-known to the skilled artisan), either consecutively with, pre- or post-conventional therapy. For example, antibodies and antigen-binding fragments or derivatives thereof of the present disclosure can be administered with a therapeutically effective dose of a neuroprotective agent and/or a therapeutically effective dose of a thrombolytic agent. In other embodiments, antibodies and antigen-binding fragments or derivatives thereof of the present disclosure are administered in conjunction with one or more additional treatment or agent. The additional treatment may be present in a single composition with the antibodies and antigen-binding fragments or derivatives of the disclosure (or the nucleic acids, vectors, etc.), or they may be administered in two different compositions simultaneously or sequentially. The dosing regimen and dosages of these aforementioned additional therapies will depend on the particular disorder being treated, the extent of the disorder and other factors familiar to the physician of skill in the art, and can be determined by the physician.

In some embodiments of methods of the disclosure, the additional or second anti-neurological- or anti-neurodegenerative-disorder therapy is a surgery, a drug, another antibody, or any combination thereof.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1: Design of Humanized Antibodies

Design of Humanization by CDR-Grafting. The mouse monoclonal antibody (mAb) 15A4-B2-E5 (IgG2b/Kappa; also known as glunomab) blocks the interaction of t-PA with the NMDAR (see WO2014/187879 for description and characterization of the mouse 15A4-B2-E5 antibody (SEQ ID NOs: 23/53)).

Humanization of mouse 15A4-B2-E5 monoclonal antibody was performed using CDR-grafting technology. The principle of this method is to reshape a human antibody containing only the complementarity determining regions (CDRs) from the mouse monoclonal antibody with the aim of reducing immunogenicity when used as a therapeutic in humans. Humanization by CDR-grafting requires that the antigen-binding residues from the mouse antibody be retained in the humanized antibody.

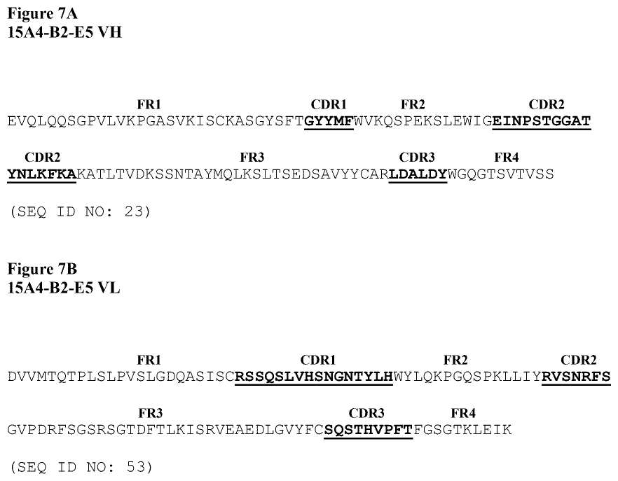

The sequences of the heavy chain variable region (VH) and light chain variable region (VL) of the mouse antibody 15A4-B2-E5 are shown in FIGS. 7A-7B respectively. The sequences of the CDRs as defined by the Kabat nomenclature are shown in bold and underlined.

The selection of human framework acceptor regions into which 15A4-B2-E5 mouse CDR regions were grafted was accomplished by searching the IMGT mouse and human V gene databases using IgBLAST, developed at NCBI to facilitate analysis of immunoglobulin V region sequences (http://www.ncbi.nlm.nih.gov/igblast/), with 15A4-B2-E5 mouse variable region sequences as input. The applied strategy was to use the human germline sequences that are natural human sequences not containing the idiosyncratic somatic mutations found in individual human antibody sequences.

The 15A4-B2-E5 mouse antibody was humanized by grafting the three CDRs, as defined by the Kabat nomenclature, from the VL into a human germline VL that was as-homologous-as-possible to the mouse antibody VL. Similarly, the three CDRs from the VH were grafted into a human germline VH that was as-homologous-as-possible to the mouse antibody VH. In addition, a few amino acid residues in the framework regions of the selected human germline variable regions were changed to the amino acid residues that were present in the mouse variable regions (so called “back-mutations”). Based upon information on the structure of immunoglobulin variable regions, the few residues in the framework regions that are known to potentially play a key role in either maintaining the CDRs in the right conformation or in VH/VL packing were identified and thus retained in the humanized versions.

For the design of CDR-grafted versions of the 15A4-B2-E5 mouse VL, two human germlines, IGKV2-30*02 and IGKV2-18*01 were selected. Human germline IGKV2-30*02 and IGKV2-18*01 both have high sequence identities of 81.0% and 80.0%, respectively, with 15A4-B2-E5 VL across the whole V gene (including CDR1 and CDR2 and 2/3 of CDR3).

The percentage of sequence identity (i.e. percentage of humanness) across the whole V genes between the 15A4-B2-E5 mouse parental monoclonal antibody and the different CDR-grafted humanized versions is presented in Table A below.

TABLE A
Percentage identity between mouse V genes or their humanized versions
with variable human germlines (V genes) used as acceptor sequences.

| | | V genes | | | | | | |
|---|---|---|---|---|---|---|---|---|
| | | Mouse | | | | | | Total |
| | | Parental | | | | Number of | | number of |
| | IMGT | 15A4- | Humanized version | | | identical | | amino |
| | Human | B2-E5 | A | B | C | residues | | acid |
| | germline | Degree of Humanness | | | | Mouse | A/B/C | residues |
| VH | IGHV1- | 61.2% | 84.7% | — | — | 60 | 83/ | 98 |
| | 2*01 | | | | | | | |
| | IGHV1- | 62.2% | 82.7% | — | — | 61 | 81/ | 98 |
| | 46*01 | | | | | | | |
| | IGHV4- | 50.5% | 83.7% | 84.7% | 83.7% | 49 | 82/83/82 | 98 |
| | 34*01 | | | | | | | |
| | IGHV4- | 52.6% | 84.7% | 83.7% | — | 51 | 83/82 | 98 |
| | 34*09 | | | | | | | |
| VL | IGKV2- | 81.0% | 89.0% | — | — | 81 | 89/ | 100 |
| | 30*02 | | | | | | | |
| | IGKV2- | 80.0% | 88.0% | — | — | 80 | 88/ | 100 |
| | 18*01 | | | | | | | |

—: not applicable

The humanized versions (named versions A, B, C herein) for both VH and VL are conservative versions that explicitly minimize/avoid alteration of CDR residues. These versions give a similar to or better binding/potency activity than the chimeric antibody (15A4-B2-E5 mouse VH and VL fused to human constant regions) which is used as a reference molecule. In the first instance, 8 different humanized versions of mouse monoclonal antibody 15A4-B2-E5 together with the chimeric version (see details of the humanized versions in Table B below) were screened using yeast surface display technology (at Deeptope, France).

TABLE B
Humanized versions of antibodies.

| | ANTIBODY | HEAVY CHAIN | LIGHT CHAIN |
|---|---|---|---|
| 1 | 15A4-12-VHA-230-VLA | 15A4-12-VHA | 15A4-230-VLA |
| | | (SEQ ID NO: 151) | (SEQ ID NO: 34) |
| 2 | 15A4-4341-VHA-230- | 15A4-4341-VHA | |
| | VLA | (SEQ ID NO: 153) | |
| 3 | 15A4-4341-VHB-230- | 15A4-4341-VHB | |
| | VLA | (SEQ ID NO: 154) | |
| 4 | 15A4-4349-VHA-230- | 15A4-4349-VHA | |
| | VLA | (SEQ ID NO: 156) | |
| 5 | 15A4-12-VHA-218-VLA | 15A4-12-VHA | 15A4-218-VLA |
| | | (SEQ ID NO: 151) | (SEQ ID NO: 159) |
| 6 | 15A4-4341-VHA-218- | 15A4-4341-VHA | |
| | VLA | (SEQ ID NO: 153) | |
| 7 | 15A4-4341-VHB-218- | 15A4-4341-VHB | |
| | VLA | (SEQ ID NO: 154) | |
| 8 | 15A4-4349-VHA-218- | 15A4-4349-VHA | |
| | VLA | (SEQ ID NO: 156) | |
| 9 | 15A4-chimeric | 15A4-VH | 15A4-VL |
| | | (SEQ ID NO: 23) | (SEQ ID NO: 53) |

The 8 humanized versions and the chimeric version were expressed as Fab fragments at the surface of the yeast cells and their level of expression was monitored. None of the 8 humanized versions were able to be expressed at the surface of the yeast cells, whereas the chimeric version showed good expression. VH and VL mixed experiments showed that the low expression was due to the heavy chain (indeed, the VH chimeric paired with the humanized light chain version 15A4-230-VLA (SEQ ID NO: 34) exhibited good expression at the surface of the yeast cells). This result was confirmed by expressing the same 8 humanized versions as human IgG1 (at Icosagen, Estonia). Very low expression in CHO cells after transient transfection was observed, except for the chimeric version which expressed well.

In an attempt to solve the expression issue, 3 new humanized VH versions were designed. One was based on IGHV1-2*01, where it was hypothesized that Kabat residue H88 Ala (A) which was mutated to Val (V) in the human germline could be responsible for a misfolding of the humanized VH. Rather than using the human germline IGHV1-2*01, a close parent, IGHV1-46*01 was used to design the humanized version 15A4-146-VHA (SEQ ID NO: 7). The other 2 humanized versions were derived from human germline IGHV4-34; version C for IGHV4-34*01 (15A4-4341-VHC) and version B for IGHV4-34*09 (15A4-4349-VHB). These 3 new humanized versions were paired with humanized version 15A4-230-VLA (SEQ ID NO: 34) and expression at the surface of yeast cells was monitored using yeast surface display technology (at Deeptope, France). Remarkably, only the humanized 15A4-146-VHA/15A4-230-VLA (SEQ ID NO: 7/34) combination (also named as “A01”) was found to express as well, if not better than, the 15A4-chimeric version. The good expression level of 15A4-146-VHA/15A4-230-VLA as full length IgG was confirmed after transient transfection in CHO cells by Icosagen, Estonia.

### A. Heavy Chain Design

Selection of human framework acceptor VH regions. The selection of human framework acceptor VH regions into which the 15A4-B2-E5 mouse CDR regions were grafted was accomplished by searching the IMGT human VH gene database using IgBLAST (https://www.ncbi.nlm.nih.gov/igblast/igblast.cgi) with the mouse 15A4-B2-E5 VH amino acid sequence as input. Based on the sequence alignment of the Parental antibody to the human germlines, the closest matching entries were identified. The identification of the optimal human germline as acceptor was based on the following ordered criteria: sequence identity across the framework as defined by Kabat, and identity and/or compatibility of inter-chain interface residues and support loops with the canonical conformations of the Parental CDRs.

One of the important criteria in selecting a suitable human germline candidate is to be able to reach close to 85% sequence identity between this particular germline and the designed humanized version. We analysed a large number of human germline sequences and selected four human germlines as best candidates: two belonging to the human family 1, IGHV1-2*01 and IGHV1-46*01, and two belonging to the human family 4, IGHV4-34*01 and IGHV4-34*09. Thus, for the design of CDR-grafted versions of the 15A4-B2-E5 mouse VH, four human germlines, IGHV1-2*01, IGHV1-46*01, IGHV4-34*01 and IGHV4-34*09 were selected (see Table A). Human germline IGHV1-2*01 and IGHV1-46*01 have the highest sequence identity of 61.2% and 62.2% with mouse 15A4-B2-E5 VH, whereas human germline IGHV4-34*01 and IGHV4-34*09 have much lower sequence identity percentages of 50.5% and 52.6%, respectively.

In framework 4, the gene segment of the mouse 15A4-B2-E5 VH corresponding to the J gene was identified as most homologous to the mouse germline IGHJ4 gene. The mouse IGHJ4 segment gene was compared to the human J-segment genes over CDR3 and FR4, and found to be most homologous to human J4*01-segment.

### Design Using IGHV1-2*01 Human Germlines as Framework Acceptor Regions.

There is 61.2% sequence identity (60 identical residues out of a total of 98 residues in the V gene) between the 15A4-B2-E5 mouse heavy-chain variable (VH) region and the human germline immunoglobulin VH 1-2*01 (IGHV1-2*01).

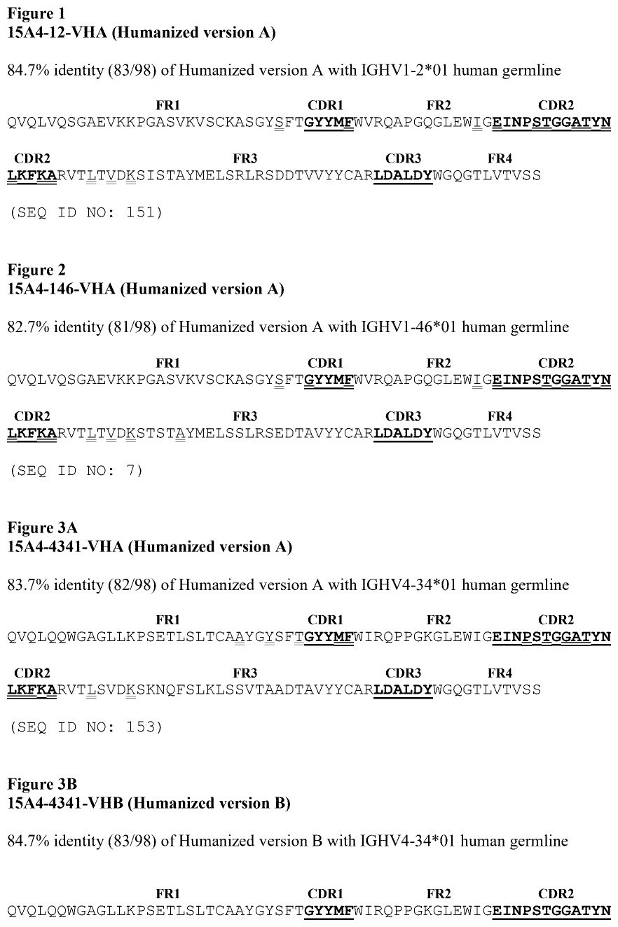

For Humanized version A, mouse CDRs as defined by the Kabat nomenclature were grafted into IGHV1-2*01. Certain framework mouse residues (non-CDR residues) were conserved from the Parental mouse 15A4-B2-E5 VH sequence because they might be structurally important for maintaining the full activity of the antibody; these residues are shown double underlined in FIG. 1. Humanized version A (15A4-B2-E5-12-VHA; also referred to as “15A4-12-VHA (Humanized version A)” (SEQ ID NO: 151)) has 84.7% identity (83 identical residues out of a total of 98 residues in the V gene) with IGHV1-2*01 human germline. The full-length amino acid sequence is shown in FIG. 1. In the framework regions and in CDRs, residues that differ between Humanized version A and human germline IGHV1-2*01 are double underlined. The CDRs are shown in bold and underlined.

### Design Using IGHV1-46*01 Human Germline as Framework Acceptor Regions.

There is 62.2% sequence identity (61 identical residues out of a total of 98 residues in the V gene) between the 15A4-B2-E5 mouse heavy-chain variable (VH) region and the human germline immunoglobulin VH-1-46*01 (IGHV1-46*01).

For Humanized version A, mouse CDRs as defined by the Kabat nomenclature were grafted into IGHV1-46*01. Certain framework mouse residues (non-CDR residues) were conserved from the Parental mouse 15A4-B2-E5 VH sequence because they might be structurally important for maintaining the full activity of the antibody; these residues are shown double underlined in FIG. 2. Humanized version A (15A4-146-VHA; SEQ ID NO: 7) has 82.7% identity (81 identical residues out of a total of 98 residues in the V gene) with IGHV1-46*01 human germline. The full-length amino acid sequence is shown in FIG. 2. In the framework regions and in CDRs, residues that differ between Humanized version A and human germline IGHV1-46*01 are double underlined. The CDRs are shown in bold and underlined.

Design using IGHV4-34*01 human germline as framework acceptor regions.

There is 50.5% identity (49 identical residues out of a total of 98 residues in the V gene) between the 15A4-B2-E5 mouse heavy-chain variable (VH) region and the human germline immunoglobulin VH 4-34*01 (IGHV4-34*01).

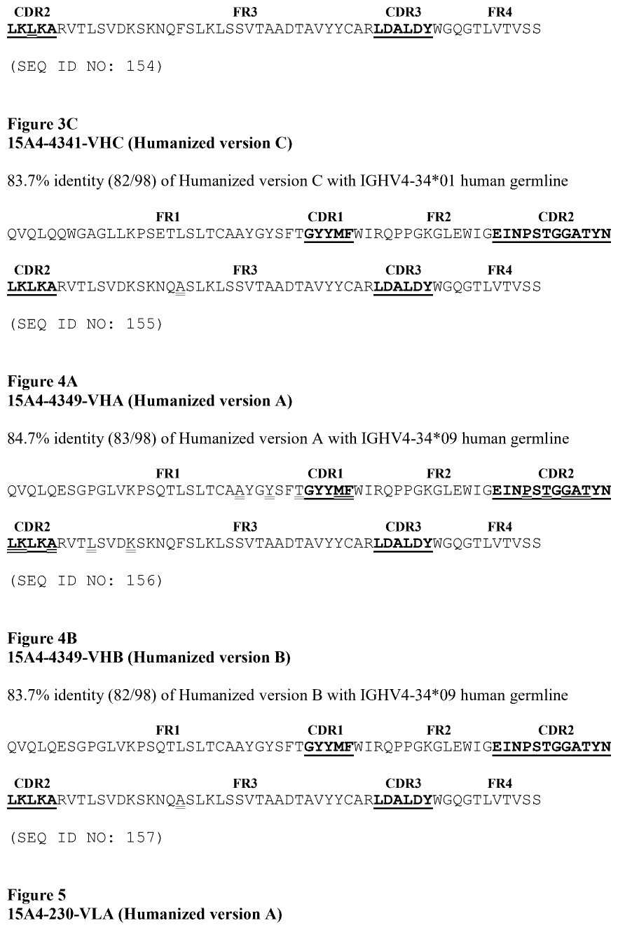

For Humanized version A, mouse CDRs as defined by the Kabat nomenclature were grafted into IGHV4-34*01. Certain framework mouse residues (non-CDR residues) were conserved from the Parental mouse 15A4-B2-E5 VH sequence because they might be structurally important for maintaining the full activity of the antibody; these residues are shown double underlined in FIG. 3A. Humanized version A (15A4-4341-VHA; SEQ ID NO: 153) has 83.7% identity (82 identical residues out of a total of 98 residues in the V gene) with IGHV4-34*01 human germline. The full-length amino acid sequence is shown in FIG. 3A. In the framework regions and in CDRs, residues that differ between Humanized version A and human germline IGHV4-34*01 are double underlined. The CDRs are shown in bold and underlined.

For Humanized version B, as compared to version A, one amino acid residue was germlined (i.e., the murine residue was substituted by the corresponding IGHV4-34*01 human germline residue). In CDR2, Kabat Phe (F) H63 was substituted with Leu (L) in order to evaluate the impact of this position on the biophysical properties of the resulting antibody. Humanized version B (15A4-4341-VHB) has 84.7% identity (83 identical residues out of a total of 98 residues in the V gene) with IGHV4-34*01 human germline. The full-length amino acid sequence is shown in FIG. 3B. The residue that differs between Humanized versions A and B is shown enlarged and in bold. The CDRs are shown in bold and underlined.

For Humanized version C, as compared to version B, one amino acid residue was germlined (i.e., the murine residue was substituted by the corresponding IGHV4-34*01 human germline residue). In framework 3 (FR3), Kabat Phe (F) H78 was back mutated to Ala (A) as found in the_Parental mouse sequence at this position in order to evaluate the impact of this position on the conformation of the CDRs. Humanized version C (15A4-4341-VHC) has 83.7% identity (82 identical residues out of a total of 98 residues in the V gene) with IGHV4-34*01 human germline. The full-length amino acid sequence is shown in FIG. 3C. The residue that differs between Humanized versions B and C is shown enlarged and in bold. The CDRs are shown in bold and underlined.

### Design Using IGHV4-34*09 Human Germline as Framework Acceptor Regions.

There is 52.6% identity (51 identical residues out of a total of 98 residues in the V gene) between the 15A4-B2-E5 mouse heavy-chain variable (VH) region and the human germline immunoglobulin VH 4-34*09 (IGHV4-34*09).

For Humanized version A, mouse CDRs as defined by the Kabat nomenclature were grafted into IGHV4-34*09. Certain framework mouse residues (non-CDR residues) were conserved from the Parental mouse 15A4-B2-E5 VH sequence because they might be structurally important for maintaining the full activity of the antibody; these residues are shown double underlined in FIG. 4A. Humanized version A (15A4-4349-VHA; SEQ ID NO: 156) has 84.7% identity (83 identical residues out of a total of 98 residues in the V gene) with IGHV4-34*09 human germline. The full-length amino acid sequence is shown in FIG. 4A. In the framework regions and in CDRs, residues that differ between Humanized version A and human germline IGHV4-34*09 are double underlined. The CDRs are shown in bold and underlined.

For Humanized version B, as compared to version A, one amino acid residue was germlined (i.e., the murine residue was substituted by the corresponding IGHV4-34*09 human germline residue). In framework 3 (FR3), Kabat Phe (F) H78 was back mutated to Ala (A) as found in the parental mouse sequence at this position, in order to evaluate the impact of this position on the conformation of the CDRs. Humanized version B (15A4-4349-VHB) has 83.7% identity (82 identical residues out of a total of 98 residues in the V gene) with IGHV4-34*09 human germline. The full-length amino acid sequence is shown in FIG. 4B. The residue that differs between Humanized versions A and B is shown enlarged and in bold. The CDRs are shown in bold and underlined.

### B. Light Chain Design

Selection of human framework acceptor VL regions. The selection of human framework acceptor VL regions into which the 15A4-B2-E5 mouse CDR regions were grafted was accomplished by searching the IMGT human VL gene database using IgBLAST with the mouse VL region amino acid sequence as input. Based on the sequence alignment of the Parental antibody to the human germlines, the closest matching entries were identified. The identification of the optimal human germline as acceptor was based on the following ordered criteria: sequence identity across the framework as defined by Kabat, and identity and/or compatibility of inter-chain interface residues and support loops with the canonical conformations of the Parental CDRs. One of the important criteria in selecting a suitable human germline candidate is to be able to reach close to 85% sequence identity between this particular germline and the designed humanized version. We analyzed a large number of human germlines and selected two human germlines, IGKV2-30*02 and IGKV2-18*01 for the design of CDR-grafted humanized versions. Both human germlines IGKV2-30*02 and IGKV2-18*01 have a very high sequence identity with mouse 15A4-B2-E5 VL with 81.0% and 80.0% (81 and 80 identical residues out of a total of 100, respectively), respectively.

The gene segment of the mouse 15A4-B2-E5 VL corresponding to the J gene is 100% identical in sequence to the mouse JK gene, IGKJ2*01. The mouse IGKJ2*01 gene segment was compared to the human JK-segment genes over CDR3 and FR4, and the human JK-segment IGKJ4*01 was found to have the highest overall sequence identity.

### Design Using IGKV2-30*02 Human Germline as Framework Acceptor Regions.

There is 81.0% identity (81 identical residues out of a total of 100 residues in the V gene) between the 15A4-B2-E5 mouse light-chain variable (VL) region and the human germline immunoglobulin VL 2-30*02 (IGKV2-30*02).

For Humanized version A, mouse CDRs as defined by the Kabat nomenclature were grafted into IGKV2-30*02. Certain mouse framework residues (non-CDR residues) were conserved from the Parental mouse 15A4-B2-E5 VL sequence because they might be structurally important for maintaining the full activity of the antibody; these residues are shown double underlined in FIG. 5. Humanized version A (15A4-230-VLA; SEQ ID NO: 34) has 89.0% identity (89 identical residues out of a total of 100 residues in the V gene) with IGKV2-30*02. The full-length amino acid sequence is shown in FIG. 5. In the framework regions and the CDRs, residues that differ between Humanized version A and human germline IGKV2-30*02 are double underlined. The CDRs are shown in bold and underlined.

### Design Using IGKV2-18*01 Human Germline as Framework Acceptor Regions.

There is 80.0% identity (80 identical residues out of a total of 100 residues in the V gene) between the 15A4-B2-E5 mouse light chain variable (VL) region and the human germline immunoglobulin VL 2-18*01 (IGKV2-18*01).

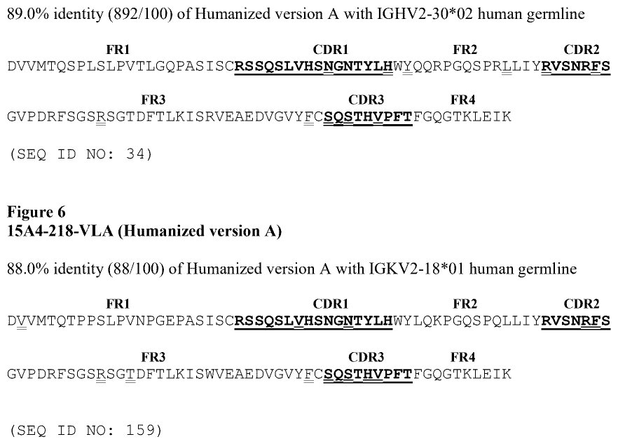

For Humanized version A, mouse CDRs as defined by the Kabat nomenclature were grafted into IGKV2-18*01. Certain mouse framework residues (non-CDR residues) were conserved from the parental mouse 15A4-B2-E5 VL sequence because they might be structurally important for maintaining the full activity of the antibody; these residues are shown double underlined in FIG. 6. Humanized version A (15A4-218-VLA) has 88.0% identity (88 identical residues out of a total of 100 residues in the V gene) with IGKV2-18*01. The full-length amino acid sequence is shown in FIG. 6. In the framework regions and the CDRs, residues that differ between Humanized version A and human germline IGKV2-18*01 are double underlined. The CDRs are shown in bold and underlined.

### Example 2: Phage Display and First-Step Screening of Antibody Variants

Affinity maturation on epitope peptide. The humanized 15A4-146-VHA/15A4-230-VLA combination (also named “A01”), which expressed well in both yeast and mammalian cells, was selected for further mutagenesis of the CDRs and affinity maturation.

### (1) Experimental Design.

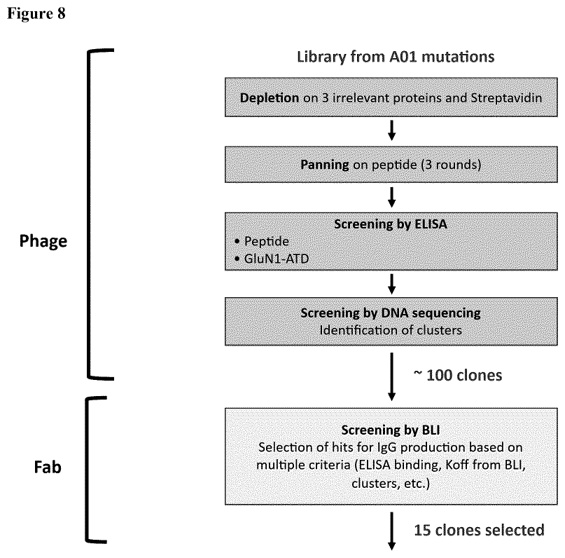

A phage library of 15A4-146-VHA/15A4-230-VLA mutations of approximately 10¹⁰ diversity was built and screened by assaying for binding to the epitope peptide, by ELISA and by DNA sequencing, to identify about 100 clones. The 100 clones were then screened by Biolayer Interferometry (BLI) and hits were selected for IgG production based on multiple criteria, including ELISA binding, k_{off} determined by BLI assay, clusters of similar sequences (mainly based on sequence identity of VH-CDR3), and the like. Specifically: phage display panning under stringent conditions designed to retain only high affinity binders was conducted, followed by initial screening of the positive clones by ELISA, sequencing of the positive clones, and estimation of affinity and kinetic binding constants by BLI for ranking of variants, using glunomab Fab as reference. A schematic diagram of the affinity maturation program by phage display is shown in FIG. 8. The antigen used for this affinity maturation program was the C-term biotinylated 15-amino-acid long peptide (172-186), which contains the glunomab epitope from the GluN1 subunit of the NMDAR. The sequence of this peptide has also been reversed to generate an irrelevant peptide (scrambled peptide) used as a negative control of binding specificity. The top ranked Fabs based on affinities were produced and purified in full human IgG1 format for further screening and sequence optimization.

### (2) Results.

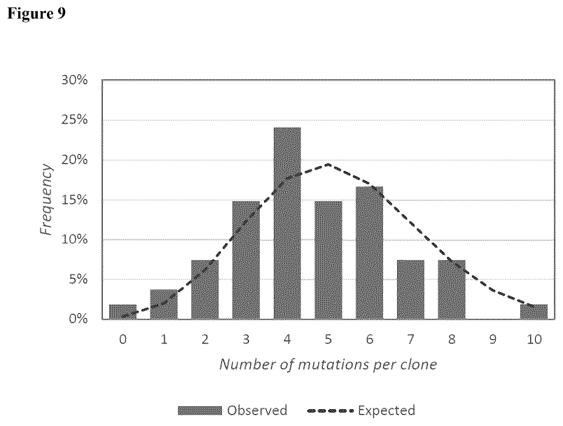

The extent of the phage library was estimated by titration to be around 7×10¹² cfu/mL. Final quality check of the library included a colony Polymerase Chain Reaction (PCR) screening and sequencing of 100 randomly picked colonies. The colony PCR results met the performance test criteria (>85% clones recombined both in frame VH and VL chains). The sequencing confirmed the proper combination diversity of VH/VL fragments, the absence of DNA contamination, the observed amino acid distributions consistent with the design of the library and the expected number of mutations per clone (FIG. 9).

The phage display selection strategy consisted of 3 rounds of panning performed in stringent conditions to promote the selection of A01 variants with increased affinity and specificity to GluN1, substituted for the screening by the synthetic epitope peptide.

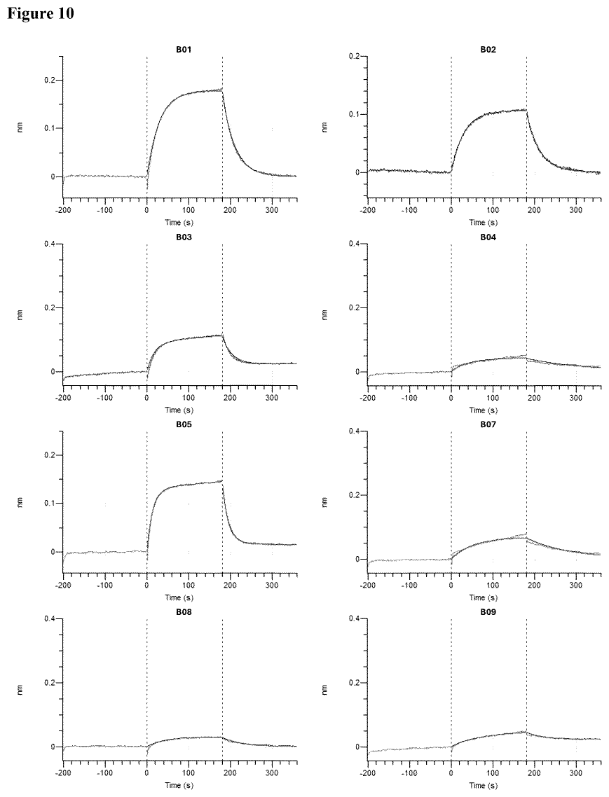

Screening of approximately 200 Fabs from the third round of panning was performed by phage-ELISA and resulted in the selection of 96 positive Fabs on the epitope peptide, among which 46 were also positive on recombinant human GluN1-ATD. These positive Fabs were then sequenced, analyzed by BLI, and ranked according to their estimated k_{off}. Among them, several Fabs displayed a k_{off} with approximately 10-fold decrease compared to A01 IgG, thus satisfying the success criteria previously established (FIG. 10).

Taken together, on the basis of ELISA, sequencing and BLI results, 15 affinity matured Fab clones (B01 to B15) derived from A01 were selected for further characterization.

### Example 3: Antibody Variant Design and Final Screening

### (1) Experimental Design.

Various assays and studies were performed to test the 15 A01 variants (B01 to B15) selected in Example 2. The different results generated, together with a fine analysis of the sequences of the variants allowed the identification of a number of amino acid mutations having a positive impact on antibody affinity, expression, biophysical properties or potency. These mutations of interest were then used to design and generate a new set of variants. This cycle of sequence design and antibody testing was repeated several times to produce new variants.

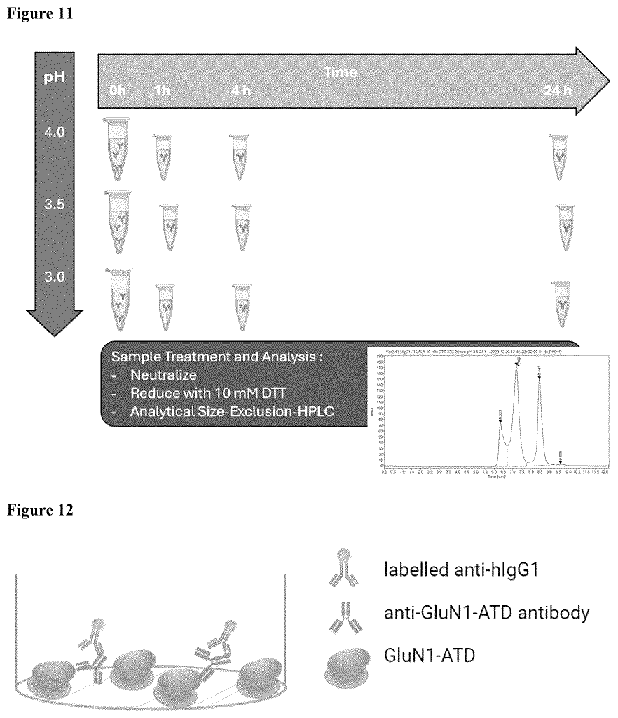

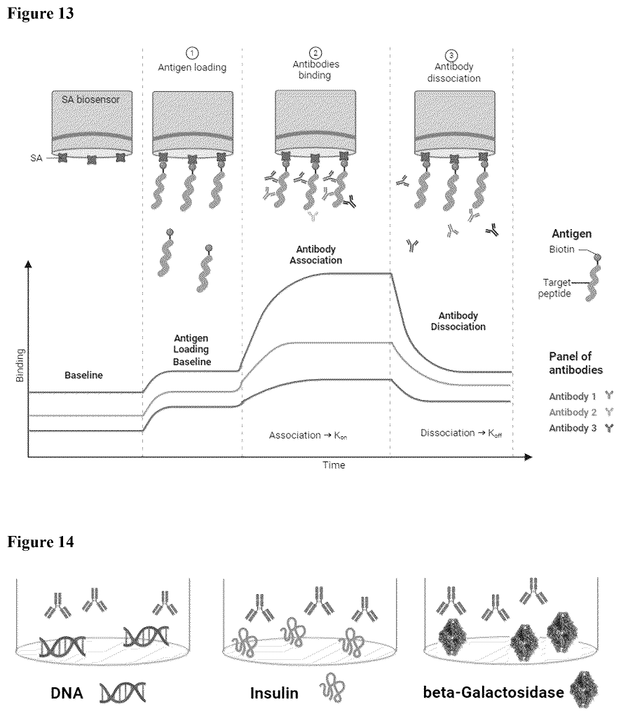

The following testing methods and criteria were used for this screening program: Manufacturability (Yield, Aggregation); Physico-chemical features (Tagg (aggregation temperature), to evaluate aggregability; Tm (melting temperature), to evaluate thermostability; Fragmentation in acid conditions, to evaluate acidity stability (FIG. 11); In vitro assays, including EC50 determination by ELISA on recombinant human GluN1-ATD to evaluate binding affinity (FIG. 12), BLI on epitope peptide to evaluate binding affinity and kinetics (FIG. 13), and ELISA on insulin, DNA and β-galactosidase, to evaluate polyreactivity (FIG. 14).

Aggregability and thermostability evaluation by Tagg and Tm determination. The melting temperature (Tm) and aggregation temperature (Tagg) of the variant antibodies were determined in duplicate by differential scanning fluorimetry (DSF) on a NanoTemper™ Prometheus NT.48. Samples were diluted in PBS at 1 mg/mL.

Acidic sensitivity evaluation by antibody fragmentation upon acid conditions. The experimental design of antibody fragmentation study upon acid conditions is shown in FIG. 11. Each antibody was incubated at 25° C., at pH3.0, pH3.5 and pH4.0 during 1 h, 4 h and 24 h. After incubation, samples were neutralized and reduced with 10 mM DTT before analysis by analytical HPLC-size exclusion chromatography.

EC50 determination by ELISA assay on GluN1-ATD proteins. The experimental design of ELISA assay on ATD protein is shown in FIG. 12. The antigen, human-ATD23-371TwinStrepTag (batch #6), was directly adsorbed on the polystyrene ELISA plates. Then, binding of the antibodies to the antigen was detected using a secondary HRP-labelled anti-human IgG. The EC50 values of each anti-GluN1-antibody were determined by testing a set of antibody dilutions (0.003 nM to 500 nM) that allowed calculation of the concentration at which only half of the ATD molecules were detected. This value reflects the relative affinity of each antibody.

BLI assay on epitope peptide. The experimental design of the BLI assay on epitope peptide is shown in FIG. 13. BioLayer Interferometry (BLI) allows the analysis of biomolecular interaction at the surface of a sensor by measuring the interferometry signal induced by this interaction. The epitope peptide (GPS_22474) was immobilized on the sensor (ligand) and the antibodies were in solution (analyte)(FIG. 13). Affinity matured anti-GluN1 variants in IgG format were tested, as well as the parental A01, on an 8-point range of concentrations from 1 nM to 1000 nM, to determine their affinity and kinetic parameters.

Polyreactivity assay by ELISA on insulin, DNA, and β-galactosidase. Experimental design of the ELISA on DNA, insulin and β-galactosidase is shown in FIG. 14. Insulin and DNA are model molecules for charge-based non-specific interaction assessment. The binding of anti-GluN1 IgG variants to β-Galactosidase, insulin, and DNA respectively, were tested by ELISA to check the levels of possible non-specific reactivity. In this assay, the DNA, insulin and beta-galactosidase were passively coated directly on separate polystyrene microplates. The remaining of the protocol was similar to the EC50 ELISA described above.

Antibody variants were tested at a concentration of 67 nM (FIG. 14). This concentration was chosen because it is above most EC50 values of the panel of antibodies to be analyzed, ensuring detection of non-specific binding while allowing a proper comparison of the hit candidate antibodies.

### (2) Results

Round 1 design and testing. In addition to the 15 clones (B01 to B15) selected from the phage display affinity maturation library described in Example 2, 7 additional variants (B16 to B22) were designed using the above-described approach, making a total of 22 A01 variants which were produced as human IgG1κ for the continuation of the screening process.

19 out of the 22 tested A01 variants had an EC50 on ATD<30 nM (at least 10 times lower than that of A01). Of these 19, 8 presented improved specificity and stability properties compared to the parental A01 (Table 8).

Round 2 design and testing. Based on the results from Round 1, a new series of 29 variants was designed (C01 to C29) and produced as human IgG1κ for the continuation of the screening process. 8 out of the new 29 tested A01 variants had an EC50 on ATD<30 nM (at least 10 times lower than that of A01). Of these, 4 presented improved specificity properties compared to the parental A01, but worse thermostability properties (Table 8).

Round 3 design and testing. Based on the results from Round 2, a new series of 26 variants was designed (D01 to D26) and produced as human IgG1κ for the continuation of the screening process.

16 out of the 26 tested A01 variants had an EC50 on ATD<30 nM (at least 10 times lower than that of A01). Of these, 5 presented an EC50 significantly lower (<10 nM), demonstrating a further improvement in binding affinity. All the clones showed improved specificity properties when compared to the parental A01 and none of them showed significantly worse stability properties (Table 8).

In total, 77 variants of A01 were generated as IgG and tested in the screening program. Results obtained with these variant antibodies are presented in Table 8 below.

TABLE 8
In vitro testing of A01 variants (human IgG1 kappa isotype).

| | | POLYREACTIVITY | | | | | | |
|---|---|---|---|---|---|---|---|---|
| | POTENCY | ELISA ON ANTIGENS | | | | | STABILITY | |
| | ELISA ON ATD | Insulin | DNA | β-gal | EXPRESSION^{a} | | | Tm |
| Compound | EC50 | (OD | (OD | (OD | Yield^{b} | Conc^{b} | Tagg | onset |
| Ref | (nM) | 450 nm) | 450 nm) | 450 nm) | (mg/mL) | (mg/mL) | (° C.) | (° C.) |
| A01 | 424.2^{d} | 0.059^{d} | 0.129^{d} | 0.300^{d} | 0.086 | 4.48 | 70.6^{d} | 62.9^{d} |
| A02 | 1223.4^{c} | 0.082 | 0.257 | 0.660 | 0.137 | 3.43 | 68.7 | 61.5 |
| A03 | 664.3 | 0.070 | 0.222 | 0.326 | 0.302 | 3.77 | 78.0 | 65.4 |
| B01 | 2.4 | 0.103 | 0.644 | 1.075 | 0.084 | 4.18 | 71.7 | 63.9 |
| B02 | 0.6 | 0.250 | 0.707 | 2.547 | 0.008 | 0.27 | Art. | 62.3 |
| B03 | 18.3 | 0.094 | 0.129 | 0.329 | 0.056 | 2.54 | 72.1 | 64.0 |
| B04 | 10.1^{c} | 0.070 | 0.102 | 0.114 | 0.056 | 3.34 | 72.8 | 64.4 |
| B05 | 5.0 | 0.604 | 0.414 | 0.923 | 0.073 | 2.62 | 69.0 | 63.1 |
| B06 | 17.7^{c} | 0.062 | 0.117 | 0.118 | 0.087 | 4.97 | 75.1 | 64.2 |
| B07 | 23.8 | 0.115 | 0.086 | 0.093 | 0.085 | 5.12 | 74.9 | 64.6 |
| B08 | 87.6^{c} | 0.075 | 0.072 | 0.093 | 0.087 | 4.74 | 71.6 | 64.7 |
| B09 | 11.7 | 0.070 | 0.079 | 0.082 | 0.084 | 5.04 | 69.7 | 64.6 |
| B10 | 14.6^{c} | 0.066 | 0.102 | 0.088 | 0.086 | 4.92 | 78.7 | 64.1 |
| B11 | 16.4 | 0.137 | 0.251 | 0.448 | 0.056 | 5.96 | 77.0 | 63.7 |
| B12 | 14.0 | 0.105 | 0.158 | 0.119 | 0.087 | 4.97 | 75.2 | 64.1 |
| B13 | 17.8 | 0.148 | 0.331 | 0.531 | 0.057 | 2.68 | 72.4 | 64.7 |
| B14 | 27.0 | 0.061 | 0.110 | 0.076 | 0.084 | 4.65 | 71.4 | 65.1 |
| B15 | 16.5 | 0.080 | 0.173 | 0.204 | 0.084 | 4.53 | 67.4 | 61.2 |
| B16 | 21.0 | 0.106 | 0.308 | 0.541 | 0.064 | 2.12 | 70.1 | 64.4 |
| B17 | 23.6 | 0.164 | 0.290 | 0.894 | 0.085 | 4.44 | 76.5 | 61.4 |
| B18 | 594.6 | 0.261 | 0.312 | 1.160 | 0.085 | 4.65 | 74.6 | 65.0 |
| B19 | 11.3 | 1.009 | 1.375 | 2.200 | 0.084 | 4.20 | 62.0 | 58.7 |
| B20 | 48.6 | 1.985 | 2.284 | 2.554 | 0.072 | 2.73 | 59.6 | 55.3 |
| B21 | 12.7 | 0.381 | 0.575 | 0.924 | 0.073 | 2.90 | 61.9 | 58.9 |
| B22 | 286.7 | 0.380 | 0.515 | 1.125 | 0.025 | 1.01 | 58.0 | 55.6 |
| C01 | 588.4 | 0.063 | 0.203 | 0.137 | 0.295 | 3.97 | 79.0 | 64.8 |
| C02 | 95.8^{c} | 0.067^{c} | 0.136^{c} | 0.260^{c} | 0.194 | 4.30 | 71.1^{c} | 64.9^{c} |
| C03 | 382.1 | 0.110 | 0.207 | 0.521 | 0.168 | 3.74 | 68.3 | 64.0 |
| C04 | 20.5^{c} | 0.058 | 0.158 | 0.093 | 0.192 | 4.81 | 71.0 | 63.9 |
| C05 | 740.8 | 0.069 | 0.207 | 0.126 | 0.154 | 3.86 | 68.3 | 63.3 |
| C06 | 33.0^{c} | 0.060 | 0.122 | 0.089 | 0.199 | 4.43 | 75.5 | 64.0 |
| C07 | 39.3^{c} | 0.062 | 0.123 | 0.171 | 0.223 | 4.95 | 71.3 | 64.8 |
| C08 | 55.2^{c} | 0.052 | 0.127 | 0.099 | 0.166 | 3.69 | 71.0 | 65.2 |
| C09 | 44.3 | 0.056 | 0.136 | 0.119 | 0.182 | 4.05 | 71.1 | 65.0 |
| C10 | 89.7 | 0.059 | 0.131 | 0.170 | 0.202 | 4.49 | 71.5 | 65.0 |
| C11 | 174.8 | 0.050 | 0.102 | 0.080 | 0.194 | 4.30 | 71.3 | 65.3 |
| C12 | 98.6 | 0.056 | 0.091 | 0.108 | 0.198 | 4.39 | 72.0 | 64.9 |
| C13 | 182.5 | 0.053 | 0.101 | 0.248 | 0.225 | 4.99 | 71.1 | 65.4 |
| C14 | 37.2 | 0.402 | 0.179 | 1.089 | 0.253 | 3.16 | 64.5 | 61.2 |
| C15 | 4.1 | 0.411 | 0.403 | 2.546 | 0.085 | 1.06 | 59.6 | 55.8 |
| C16 | 1983.0^{c} | 0.048 | 0.124 | 0.100 | 0.364 | 4.55 | 68.5 | 63.6 |
| C17 | 128.7 | 0.073 | 0.314 | 0.311 | 0.190 | 2.38 | 63.1 | 60.8 |
| C18 | 5.4^{c} | 0.056 | 0.137 | 0.088 | 0.306 | 3.83 | 66.2 | 62.9 |
| C19 | 463.2 | 0.064 | 0.254 | 0.191 | 0.170 | 2.13 | 61.4 | 59.7 |
| C20 | 163.7^{c} | 0.053 | 0.090 | 0.107 | 0.372 | 4.65 | 62.3 | 59.7 |
| C21 | 21.4 | 0.073 | 0.313 | 0.747 | 0.102 | 1.28 | 57.4 | 54.2 |
| C22 | 1897.0^{c} | 0.058 | 0.144 | 0.157 | 0.355 | 4.44 | 72.3 | 65.2 |
| C23 | 640.3 | 0.070 | 0.302 | 0.205 | 0.266 | 3.32 | 69.2 | 64.7 |
| C24 | 111.0^{c} | 0.056 | 0.109 | 0.167 | 0.278 | 3.48 | 66.6 | 63.5 |
| C25 | 24.7 | 0.066 | 0.196 | 0.445 | 0.166 | 2.08 | 62.0 | 59.6 |
| C26 | 68.2 | 0.147 | 0.158 | 0.729 | 0.314 | 3.93 | 69.3 | 64.3 |
| C27 | 16.2 | 0.252 | 0.299 | 2.007 | 0.167 | 2.09 | 63.7 | 61.0 |
| C28 | 13.8^{c} | 0.050^{c} | 0.122^{c} | 0.146^{c} | 0.325^{c} | 4.20^{c} | 69.9 | 64.4 |
| C29 | 462.3 | 0.066 | 0.176 | 0.199 | 0.295 | 3.81 | 66.7 | 63.1 |
| D01 | 5.2 | 0.050 | 0.270 | 0.197 | 0.253 | 3.39 | 69.0 | 64.9 |
| D02 | 23.6 | 0.063 | 0.201 | 0.241 | 0.150 | 1.86 | 65.2 | 62.2 |
| D03 | 3.2 | 0.051 | 0.172 | 0.187 | 0.152 | 2.08 | 61.8 | 60.3 |
| D04 | 36.0 | 0.071 | 0.383 | 0.281 | 0.101 | 1.28 | 62.0 | 61.0 |
| D05 | 191.6 | 0.065 | 0.215 | 0.283 | 0.151 | 1.87 | 60.8 | 58.9 |
| D06 | 16.3 | 0.053 | 0.148 | 0.160 | 0.151 | 1.84 | 66.9 | 64.5 |
| D07 | 236.9 | 0.068 | 0.222 | 0.313 | 0.151 | 1.84 | 63.8 | 63.4 |
| D08 | 22.8 | 0.056 | 0.170 | 0.172 | 0.203 | 2.37 | 65.0 | 63.6 |
| D09 | 18.9 | 0.058 | 0.214 | 0.213 | 0.152 | 1.88 | 63.0 | 62.6 |
| D10 | 48.5 | 0.052 | 0.136 | 0.170 | 0.151 | 1.95 | 69.1 | 64.7 |
| D11 | 3.5 | 0.050 | 0.108 | 0.168 | 0.250 | 3.36 | 66.1 | 62.8 |
| D12 | 47.4 | 0.058 | 0.201 | 0.252 | 0.201 | 2.51 | 67.7 | 65.2 |
| D13 | 20.2 | 0.053 | 0.160 | 0.231 | 0.254 | 2.91 | 73.9 | 65.0 |
| D14 | 16.3 | 0.047 | 0.105 | 0.196 | 0.304 | 3.50 | 71.1 | 65.4 |
| D15 | 11.8 | 0.049 | 0.122 | 0.196 | 0.252 | 3.16 | 71.8 | 64.7 |
| D16 | 26.2 | 0.052 | 0.096 | 0.178 | 0.250 | 3.56 | 70.2 | 65.1 |
| D17 | 3.4 | 0.055 | 0.150 | 0.114 | 0.253 | 3.38 | 66.5 | 63.9 |
| D18 | 1.2 | 0.055 | 0.145 | 0.137 | 0.250 | 3.23 | 66.1 | 63.2 |
| D19 | 33.4 | 0.057 | 0.137 | 0.130 | 0.254 | 3.00 | 70.9 | 64.9 |
| D20 | 21.2 | 0.055 | 0.174 | 0.125 | 0.200 | 2.64 | 68.4 | 64.6 |
| D21 | 30.9 | 0.058 | 0.145 | 0.189 | 0.204 | 2.38 | 66.1 | 62.5 |
| D22 | 35.9 | 0.055 | 0.161 | 0.214 | 0.203 | 2.43 | 64.8 | 61.6 |
| D23 | 245.0 | 0.066 | 0.135 | 0.205 | 0.203 | 2.37 | 71.9 | 64.4 |
| D24 | 390.4 | 0.060 | 0.105 | 0.202 | 0.203 | 2.48 | 71.8 | 64.1 |
| D25 | 46.6 | 0.055 | 0.095 | 0.201 | 0.201 | 2.80 | 73.4 | 64.6 |
| D26 | 25.6 | 0.053 | 0.125 | 0.274 | 0.201 | 2.52 | 72.0 | 64.4 |

^{a}transient expression in CHO;
^{b}purified product;
^{c}mean value resulting from testing in different runs.

Final selection. From the 77 variants generated and tested, a selection of 5 variants was made: (1) C28 presented a double digit EC50 of 13.8 nM, a good polyreactivity profile and a similar Tagg and improved Tm compared to A01. (2) D11 presented a low single digit EC50 of 3.5 nM, a very good polyreactivity profile, and similar Tagg and Tm compared to A01. It has the same sequence as D01 except for VH position H58 which has an E rather than D for D01. Both variants had similar properties. The E at H58 avoids creating a potential isomerisation DY motif (3) D14 presented a low double digit EC50 of 16.3 nM, a very good polyreactivity profile and a higher Tagg and Tm compared to A01. This variant has the parental T at H58 not creating a potential deamidation motif (4) D15 presented a low double digit EC50 of 11.8 nM, a very good polyreactivity profile and a higher Tagg and Tm compared to A01. This variant has the parental T at H58 not creating a potential deamidation motif. This variant has the same sequence as D14 except for VL position L96 which is a Y whereas D14 has the parental F. (5) D17 presented a low single digit EC50 of 3.4 nM, a good polyreactivity profile, and a similar Tagg and Tm compared to A01.

All 5 variants displayed biological efficacy in the in vivo NMDA lesion mouse model of excitotoxicity (see Example 4 below).

The 5 variants were expressed as human IgG1κ with the LALA mutation (Leucine 234 and 235 mutated to Alanine; standard Fc silencing mutations). C28 was also expressed as human IgG4κ isotype. The 5 hIgG1κ-LALA variants were tested in a final round of in vitro assays (results are shown in Table 9, Table 10 and Table 11). Values obtained for each variant were similar to what had previously been observed, showing that the isotype switch (human IgG1 to IgG1-LALA) did not affect thermostability.

For C28, a lower Tm Onset was observed with the hIgG4 isotype in comparison to the hIgG1 isotype. Overall, in the test conditions (1 mg/mL in PBS pH7,4), no thermostability issues were detected (Table 9).

TABLE 9
5 selected A01 variants testing for thermostability.

| Sample | Tm Onset | Tm1 (~CH2) | Tm3 (~CH3) | Tagg |
|---|---|---|---|---|
| C28 hIgG4κ | 61.7 | 68.5 | 82.4 | 69.7 |
| C28 hIgG1κ-LALA | 64.6 | 70.0 | 81.9 | 69.9 |
| D11 hIgG1κ-LALA | 63.0 | 67.4 | 81.6 | 66.1 |
| D14 hIgG1κ-LALA | 65.1 | 71.2 | 81.8 | 71.3 |
| D15 hIgG1κ-LALA | 63.9 | 71.7 | 81.8 | 72.7 |
| D17 hIgG1κ-LALA | 63.2 | 67.8 | 81.6 | 66.8 |

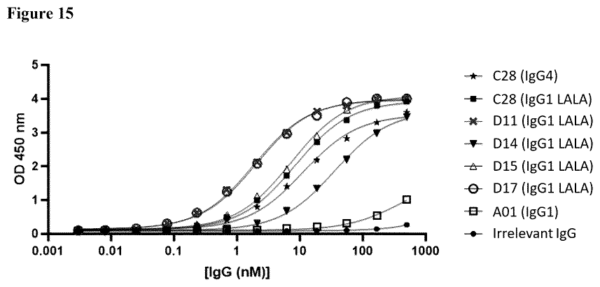

The A01 EC50 on ATD was estimated at 416.6 nM (estimate, as no top plateau was reached in the tested range of concentrations), which was consistent with previous measurements. The EC50 values of the 6 newly produced variant IgGs were consistent with the ones previously determined for the respective hIgG1 variants. The isotype switch did not affect antibody binding on ATD and affinity ranking was confirmed (FIG. 15; Table 10).

TABLE 10
EC50 values for binding to ATD for selected A01 variants.

| | | C28 | D11 | D14 | D15 | D17 | A01 |
|---|---|---|---|---|---|---|---|
| | C28 | hIgG1- | hIgG1- | hIgG1- | hIgG1- | hIgG1- | hIgG1- |
| | hIgG4 | LALA | LALA | LALA | LALA | LALA | LALA |
| EC50 | 11.2 | 8.4 | 1.8 | 35.9 | 7.2 | 2.0 | >400 |
| (nM) | | | | | | | (no |
| | | | | | | | fitting) |

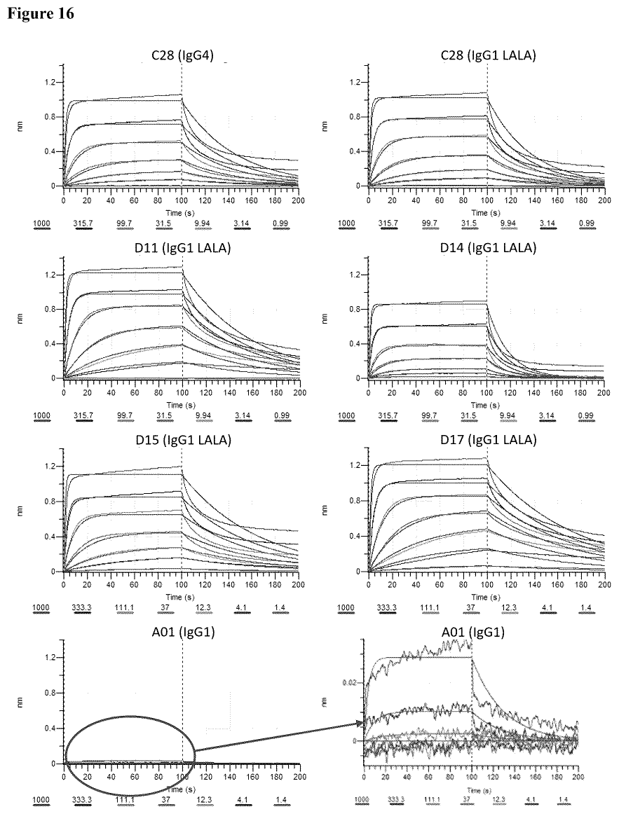

Overall, a clear improvement in affinity was observed for the selected variants in comparison with the parental A01. The tested variants presented a high constant of association (kₒₙ), but also a quite high constant of dissociation (k_{of}). The 3 clones_presenting the lowest K_{D} were D11, D15 and D17. Overall, the tested variants displayed similar binding properties to ATD (FIG. 16; Table 11).

TABLE 11
Binding properties for binding to ATD for selected A01 variants.

| Sample | K_{D} (nM) | kₒₙ (1/Ms) | k_{off} (1/s) | R² |
|---|---|---|---|---|
| C28 hIgG4κ | 32.5 | 6.6E05 | 2.1E−02 | 0.9791 |
| C28 hIgG1κ-LALA | 36.4 | 7.5E05 | 2.7E−02 | 0.9824 |
| D11 hIgG1κ-LALA | 24.9 | 7.0E05 | 1.7E−02 | 0.9822 |
| D14 hIgG1κ-LALA | 70.6 | 7.9E05 | 5.6E−02 | 0.9780 |
| D15 hIgG1κ-LALA | 16.0 | 9.6E05 | 1.5E−02 | 0.9744 |
| D17 hIgG1κ-LALA | 19.8 | 6.9E05 | 1.4E−02 | 0.9823 |
| A01 hIgG1κ | * | * | * | * |

* not determined due to low affinity

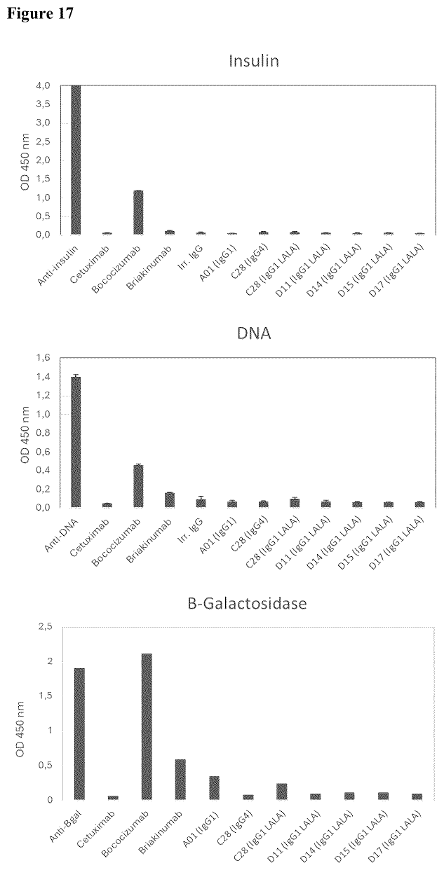

None of the tested variants showed signs of binding to insulin and DNA (ELISA assay). On β-galactosidase, all variants showed a lower signal than the parental A01 (considered a weak unspecific binding). Overall, no polyreactivity signal was observed with the tested variants (FIG. 17).

### Example 4: Efficacy of Humanized mAbs in an Animal NMDA Lesion Model

In vivo efficacy of humanized mAbs to reduce NMDA-dependent brain lesion volume in an animal model. Affinity-matured antibody variants were tested for their ability to reduce NMDA-dependent brain lesion volume in a mouse model.

In the pathophysiology of neurological disorders including multiple sclerosis, Parkinson's disease and ischemic stroke, overexpressed tissue plasminogen activator (t-PA) binding to the GluN1 subunit of NMDAR induces an increased permeability of the blood-brain barrier via NMDA receptor-mediated signals. The interaction between t-PA and the GluN1 subunit of NMDAR triggers NMDAR overactivation, leading to degradation of tight junction proteins which elicits BBB disruption, favors hemorrhagic transformations, and allows immune cells and toxic molecules to infiltrate the brain parenchyma. These mechanisms of neuroinflammation coupled with a t-PA-dependent neuronal excitotoxicity lead to neurodegeneration. NMDAR-dependent overactivation appears to be one of the primary causes of neuronal death and is mimicked by the NMDA lesion model (NMDA injection in the brain) used in the present Example.

Without wishing to be limited by theory, it is believed that anti-GluN1 antibodies of the present disclosure can act, at least in part, by preventing the interaction of t-PA with NMDAR, thereby preserving physiological function(s) of NMDAR while halting downstream detrimental pathways (Lesept, F. et al., 2016, Cell Death Dis. 2016, 7(11):e2466; Macrez, R. et al., 2016, Brain, 139(Pt 9):2406-19; Mehra, A. et al., 2020, J Neurosci., 40(8):1778-87). This effect can protect the brain from further excitotoxicity and neuroinflammatory responses, and subsequent neurodegenerative cascades.

In the present Example, we studied and compared the efficacy of anti-GluN1 antibody variants generated during the maturation program described in Examples 2 and 3 above. Efficacy was evaluated by the ability to prevent NMDA-dependent brain lesion amplified by a co-injection of recombinant t-PA (rt-PA). The test proteins were A01, the original humanized version of the antibody, and the variants B04, B06, B08, B10, C08, C18, C28, D11, D14, D15, and D17, all as human IgG1 isotype.

### (1) Experimental Design.

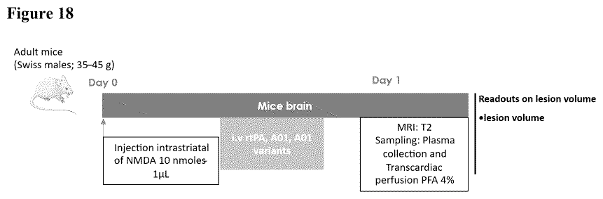

Male Swiss mice were anesthetized during surgery and animal health and well-being were monitored with extra care in the 24 hours post-surgery (FIG. 18). Striatal injection of NMDA was done using a micropipette and followed by an intravenous injection of the test proteins (5 mg/kg) and rt-PA (10 mg/kg). After 24 hours, lesion volume was determined by T2-weighted magnetic resonance imaging (MRI). In this model, rt-PA increases lesion volume, and this effect is prevented by anti-GluN1 antibodies (Lesept et al, 2016).

### (2) Results.

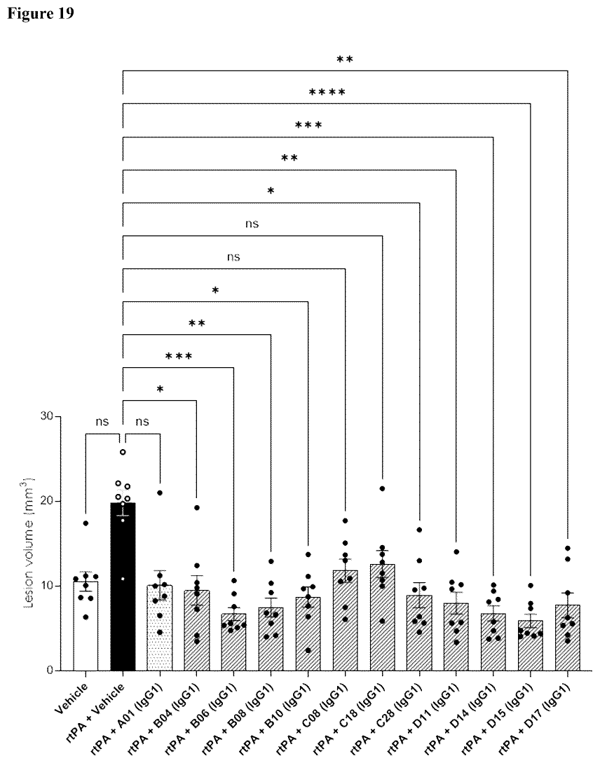

Results are shown in FIG. 19. MRI analyses show a significant decrease of the lesion volume in groups rt-PA+B04 (52% of decrease; 9.46 mm³); rt-PA+B06 (66% of decrease; 6.67 mm³); rt-PA+B08 (62% of decrease; 7.44 mm³); rt-PA+B10 (56% of decrease; 8.69 mm³); rt-PA+C28 (55% of decrease; 8.90 mm³); rt-PA+D11 (60% of decrease; 7.94 mm³); rt-PA+D14 (66% of decrease; 6.75 mm³); rt-PA+D15 (70% of decrease; 5.87 mm³); rt-PA+D17 (61% of decrease; 7.70 mm³); when compared with rt-PA alone (19.83 mm³).

Overall, all anti-GluN1 variants except C08 and C18 demonstrated a significant decrease of the brain lesion in this mice model in vivo.

### Example 5: Efficacy of Humanized mAbs in an Animal NMDA Lesion Model

### (1) Experimental Design

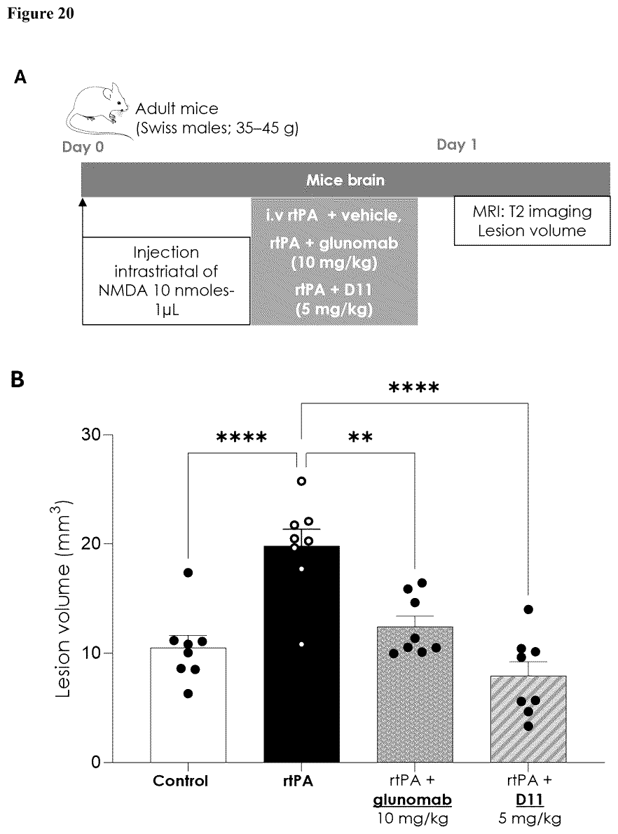

Male Swiss mice were anesthetized during the surgery and the animal health and well-being are monitored with extra care in the following 24 hours post-surgery. The procedure consisted in the striatal injection of NMDA using a micropipette followed by an intravenous injection of the antibodies (D11 or glunomab) and rtPA (10 mg/kg). After 24 hours, a lesion volume was determined by T2-weighted magnetic resonance imaging (MRI) (see FIG. 20A). In this model, rtPA increases the lesion volume and this effect is expected to be prevented by each antibody (Lesept et al, 2016).

### (2) Results

Results are shown in FIG. 20B. MRI analyses show a significant decrease of the lesion volume in, both, the rt-PA+glunomab treated (37% of decrease; 12.44 mm³) and the rt-PA+D11 treated groups (60% of decrease; 7.95 mm³), when compared to the rt-PA group group (19.83 mm³). Overall, the D11 group exhibits a more significant decrease in lesion volume, in spite of a lower dose (5 mg/kg for D11 vs. 10 mg/kg for glunomab).

### Example 6: Assessment of the Conformation Change of the NMDA Receptor (NMDAR) in a Fluorescence-Based Assay (G.Validation®)

### (1) Experimental Design

G.Validation® assay is a G.CLIPS biotech (https://gclips-biotech.com/) proprietary fluorescence-based assay that assesses conformational change of the receptor upon interaction with various ligands. The same assay was validated in previous academic studies, e.g. in Montemagno et al. (J. Exp Clin Cancer Res (2024) 43:86). Briefly, recombinant GluN1 and GluN2B subunits are produced and purified from HEK cells using G.CLIPS proprietary mixes. The NMDAR is then reconstituted in vitro and stabilized in detergent buffers containing lipids mimicking the lipidic composition of the brain. The receptor is then labelled with a non-modifying probe allowing the detection of a conformational change of the receptor upon addition of a ligand/antibody. Indeed, the probe's emission maximum wavelength (λmax) shifts according to conformational changes of the receptor. Thus, kinetics of these conformational changes of the receptor can be followed by monitoring λmax shift after addition of a ligand/antibody over time. We can thus determine the on-target effect of a ligand/antibody and determine its pharmacological profile. Kinetics of 300 nM labelled NMDAR were monitored in presence of its endogenous agonists glycine and glutamate at saturating concentration of 1 mM, different concentrations of glunomab (500 μg/mL to 0.5 μg/mL) or D11 antibody (50 μg/mL to 0.5 μg/mL) and human Tissue-type plasminogen activator (tPA) at 300 nM. Kinetics were also followed using PBS as a negative control. All emission spectra were registered for 30 min after addition of each ligand/antibody.

### (2) Results

As a first control experiment, the addition of NMDAR co-agonists glycine and glutamate (1 mM) induces a conformational change of NMDAR. PBS serves as a negative control, with no observable conformational change. Human tPA (300 nM) alone triggers a conformational change of NMDAR, indicating binding to NMDAR. As a second control experiment, a control isotype antibody (anti-HEL IgG1 LALA) is added which does not bind to NMDAR and does not prevent t-PA binding to NMDAR. Accordingly, no modulation of the λmax shift of fluorescence is observed, indicating no binding to NMDAR. In the presence of control antibody, the addition of human t-PA induces a conformational change of NMDAR, indicating binding to NMDAR, as expected.

The effect of glunomab is then investigated. The addition of glunomab does not induce a strong conformational change at the receptor, which does not imply absence of binding, but rather reflects a potentially low affinity for NMDAR—lower than that of the D11 antibody. At 100 μg/mL (molar ratio 2:1 glunomab:tPA), a conformational change is observed upon tPA addition, indicating that glunomab does not prevent tPA binding at this ratio. At 250 μg/mL (5:1 ratio), only a slight conformational change is seen, suggesting partial inhibition of tPA binding. At 500 μg/mL (10:1 ratio), no conformational change is observed upon tPA addition, indicating that glunomab fully prevents tPA binding to NMDAR at this ratio. At higher doses of glunomab—i.e., at increased glunomab:tPA ratios (20:1, 50:1, and 100:1)—no conformational change is observed upon tPA addition, indicating that glunomab fully prevents tPA binding only when present in large excess (≥20-fold). On the contrary, at higher dose of tPA—i.e., at decreased glunomab:tPA ratios (1:1, 1:5, 1:10, 1:20, 1:50, and 1:100), it would appear that the antibody has no effect on preventing tPA binding to NMDAR. The addition of tPA, in each case, induces a conformational change, indicating binding to NMDAR despite glunomab presence.

The effect of the D11 antibody is then investigated. When the D11 antibody is added, a conformational change is observed confirming a binding of D11 to NMDAR. A smaller conformational change can be observed at lower concentrations of D11, as expected. When tPA is subsequently added, the conformational change—and therefore tPA binding to NMDAR is prevented when the D11 concentration is up to 10 times lower than tPA (D11:tPA ratios of 1:1, 1:5, or 1:10). At higher ratios (1:20, 1:50 and 1:100) a conformational change is observed suggesting tPA binding.

Based on the above results, it can be concluded that D11 has superior affinity and efficacy when compared to glunomab, for preventing t-PA binding to NMDAR. Also, while glunomab only achieves full inhibition when present at a ≥10:1 molar excess over tPA, D11 remains effective even when tPA is in 10-fold excess (inversed 1:10 ratio). This corresponds to a 100-fold difference in the dose required to reach comparable functional efficacy—a two-log improvement in potency.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and amino acid sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the present invention described herein. Such equivalents are intended to be encompassed by the following claims.

### What is Disclosed is:

Item 1. A humanized anti-GluN1 antibody or a fragment or derivative thereof, wherein said antibody, fragment or derivative is specific for the GluN1 subunit of the N-methyl-D-aspartate receptor (NMDAR), wherein said antibody, fragment or derivative inhibits interaction between the NMDAR and t-PA and/or inhibits NMDAR activation by t-PA;
optionally, wherein said antibody, fragment or derivative is capable of specifically binding to an epitope represented by the amino acid sequence EGRAAQKRLETLLEE (SEQ ID NO: 182) or AQKRL (SEQ ID NO: 183);
optionally, wherein said antibody, fragment or derivative competes for binding with an antibody produced by a deposited hybridoma which is 15A4-B2-E5, 15A4-B2-F3, 15A4-B2, 6C9-A3, 6C9-A3-F4, or 6C9-A3-F6.
   Item 2. The humanized anti-GluN1 antibody, fragment or derivative of item 1, wherein said antibody, fragment or derivative comprises:
(a) a heavy chain variable region comprising: a CDR-H1 comprising or consisting of the amino acid sequence shown in SEQ ID NO: 79 or 82, a CDR-H2 comprising or consisting of the amino acid sequence shown in SEQ ID NO: 80, 92, 94 or 95, a CDR-H3 comprising or consisting of the amino acid sequence shown in SEQ ID NO: 81; or one or more CDR(s) having an amino acid sequence selected from the group consisting of SEQ ID NOs: 79-82, 92, 94 and 95 which is substituted at one or two amino acid positions or which contains a substitution of one or more amino acids, and which is functionally equivalent to the corresponding unaltered CDR(s);
(b) a light chain variable region comprising: a CDR-L1 comprising or consisting of the amino acid sequence shown in SEQ ID NO: 101, 128 or 132, a CDR-L2 comprising or consisting of the amino acid sequence shown in SEQ ID NO: 102 or 105, and a CDR-L3 comprising or consisting of the amino acid sequence shown in SEQ ID NO: 103, 106 or 117; or one or more CDR(s) having an amino acid sequence selected from the group consisting of SEQ ID NOs: 101-103, 105, 106, 117, 128, and 132 which is substituted at one or two amino acid positions or which contains a substitution of one or more amino acids, and which is functionally equivalent to the corresponding unaltered CDR(s);
(c) a heavy chain framework region comprising: a heavy chain FR1 comprising or consisting of the amino acid sequence shown in SEQ ID NO: 163, a heavy chain FR2 comprising or consisting of the amino acid sequence shown in SEQ ID NO: 164, a heavy chain FR3 comprising or consisting of the amino acid sequence shown in SEQ ID NO: 165, a heavy chain FR4 comprising or consisting of the amino acid sequence shown in SEQ ID NO: 166; or one or more heavy chain FR(s) having an amino acid sequence selected from the group consisting of SEQ ID NOs: 163-166 which is substituted at one or two amino acid positions or which contains a substitution of one or more amino acids or which is at least 80% identical to the amino acid sequence set forth in any one of SEQ ID NOs: 163-166, and which is functionally equivalent to the corresponding unaltered heavy chain FR(s);
and
(d) a light chain framework region comprising: a light chain FR1 comprising or consisting of the amino acid sequence shown in SEQ ID NO: 144, a light chain FR2 comprising or consisting of the amino acid sequence shown in SEQ ID NO: 145, a light chain FR3 comprising or consisting of the amino acid sequence shown in SEQ ID NO: 146, a light chain FR4 comprising or consisting of the amino acid sequence shown in SEQ ID NO: 147; or one or more light chain FR(s) having an amino acid sequence selected from the group consisting of SEQ ID NOs: 144-147 which is substituted at one or two amino acid positions or which contains a substitution of one or more amino acids or which is at least 80% identical to the amino acid sequence set forth in any one of SEQ ID NOs: 144-147, and which is functionally equivalent to the corresponding unaltered light chain FR(s).
   Item 3. The humanized antibody, fragment or derivative of any one of items 1 to 2, wherein said antibody, fragment or derivative comprises:
(a) CDR-H1 comprising or consisting of the amino acid sequence shown in SEQ ID NO: 79, CDR-H2 comprising or consisting of the amino acid sequence shown in SEQ ID NO: 80, CDR-H3 comprising or consisting of the amino acid sequence shown in SEQ ID NO: 81, CDR-L1 comprising or consisting of the amino acid sequence shown in SEQ ID NO: 101, CDR-L2 comprising or consisting of the amino acid sequence shown in SEQ ID NO: 102, and CDR-L3 comprising or consisting of the amino acid sequence shown in SEQ ID NO: 103;
(b) CDR-H1 comprising or consisting of the amino acid sequence shown in SEQ ID NO: 82, CDR-H2 comprising or consisting of the amino acid sequence shown in SEQ ID NO: 80, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106;
(c) CDR-H1 comprising or consisting of the amino acid sequence shown in SEQ ID NO: 82, CDR-H2 comprising or consisting of the amino acid sequence shown in SEQ ID NO: 92, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106;
(d) CDR-H1 comprising or consisting of the amino acid sequence shown in SEQ ID NO: 82, CDR-H2 comprising or consisting of the amino acid sequence shown in SEQ ID NO: 94, CDR-H3 comprising or consisting of the amino acid sequence shown in SEQ ID NO: 81, CDR-L1 comprising or consisting of the amino acid sequence shown in SEQ ID NO: 128, CDR-L2 comprising or consisting of the amino acid sequence shown in SEQ ID NO: 105, and CDR-L3 comprising or consisting of the amino acid sequence shown in SEQ ID NO: 106;
(e) CDR-H1 comprising or consisting of the amino acid sequence shown in SEQ ID NO: 82, CDR-H2 comprising or consisting of the amino acid sequence shown in SEQ ID NO: 94, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 117; or
(f) CDR-H1 comprising or consisting of the amino acid sequence shown in SEQ ID NO: 82, CDR-H2 comprising or consisting of the amino acid sequence shown in SEQ ID NO: 95, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106.
   Item 4. The humanized antibody, fragment or derivative of any one of items 1 to 3, wherein said antibody, fragment or derivative comprises or consists of any one of A01, B16, B01, B02, B03, B04, B05, B06, B07, B08, B09, B10, B11, B12, B13, B14, B15, B17, B18, B19, B20, B21, B22, C01, C02, C03, C04, C05, C06, C07, C08, C09, C10, C11, C12, C13, C14, C15, C16, C17, C18, C19, C20, C21, C22, C23, C24, C25, C26, C27, C28, C29, D01, D02, D03, D04, D05, D06, D07, D08, D09, D10, D11, D12, D13, D14, D15, D16, D17, D18, D19, D20, D21, D22, D23, D24, D25, or D26; or a variant sequence thereof which differs by only one, two or three amino acids, or which has at least or about 85% sequence identity thereto.
   Item 5. The humanized antibody, fragment or derivative of any one of items 1 to 4, wherein said antibody, fragment or derivative is capable of binding to at least a portion of a region of an NMDA receptor that interacts with tissue-type plasminogen activator (t-PA) and wherein said binding inhibits the interaction between the NMDA receptor and t-PA, such that at least one function of the NMDA receptor activated by t-PA is selectively inhibited.
   Item 6. The humanized antibody, fragment or derivative of any one of items 1 to 5, wherein said antibody, fragment or derivative is a monoclonal antibody, or a fragment or derivative thereof.
   Item 7. The humanized antibody, fragment or derivative of any one of items 4 to 6, wherein said antibody, fragment or derivative comprises:
(i) a heavy chain variable region comprising the amino acid sequence of any one of SEQ ID NOs: 7-22 or 25-33; and/or
(ii) a light chain variable region comprising the amino acid sequence of any one of SEQ ID NOs: 34-52 or 54-78.
   Item 8. The humanized antibody, fragment or derivative of any one of items 1 to 7, wherein said antibody, fragment or derivative comprises:
a heavy chain variable region comprising or consisting of the amino acid sequence of SEQ ID NO: 7, and a light chain variable region comprising or consisting of the amino acid sequence of SEQ ID NO: 34;
a heavy chain variable region comprising or consisting of the amino acid sequence of SEQ ID NO: 12, and a light chain variable region comprising or consisting of the amino acid sequence of SEQ ID NO: 65;
a heavy chain variable region comprising or consisting of the amino acid sequence of SEQ ID NO: 14, and a light chain variable region comprising or consisting of the amino acid sequence of SEQ ID NO: 65;
a heavy chain variable region comprising or consisting of the amino acid sequence of SEQ ID NO: 17, and a light chain variable region comprising or consisting of the amino acid sequence of SEQ ID NO: 74;
a heavy chain variable region comprising or consisting of the amino acid sequence of SEQ ID NO: 17, and a light chain variable region comprising or consisting of the amino acid sequence of SEQ ID NO: 75;
a heavy chain variable region comprising or consisting of the amino acid sequence of SEQ ID NO: 32, and a light chain variable region comprising or consisting of the amino acid sequence of SEQ ID NO: 65.
   Item 9. The antibody, fragment or derivative of any one of items 1 to 7, wherein said antibody, fragment or derivative comprises:
(i) a heavy chain comprising or consisting of the amino acid sequence of SEQ ID NO: 7; and/or
(ii) a light chain comprising or consisting of the amino acid sequence of SEQ ID NO: 34.
   Item 10. A nucleic acid encoding the antibody, fragment or derivative of any one of items 1 to 9.
   Item 11. An expression cassette or vector comprising the nucleic acid according to item 10, optionally comprising a promoter operatively connected with said nucleic acid.
   Item 12. A host cell comprising the nucleic acid of item 10 or the expression cassette or vector of item 11.
   Item 13. A pharmaceutical composition comprising the antibody, fragment or derivative of any one of items 1 to 9, the nucleic acid of item 10, the expression cassette or vector of item 11, or the host cell of item 12, and a pharmaceutically acceptable carrier, diluent or excipient, optionally wherein the pharmaceutical composition is suitable for administration by injection, such as subcutaneously, intramuscularly, intravenously, or intraperitoneally, or orally.
   Item 14. The antibody, fragment or derivative of any one of items 1 to 9, the nucleic acid of item 10, the expression cassette or vector of item 11, the host cell of item 12, or the pharmaceutical composition of item 13, for use in medicine.
   Item 15. The antibody, fragment or derivative of any one of items 1 to 9, the nucleic acid of item 10, the expression cassette or vector of item 11, the host cell of item 12, or the pharmaceutical composition of item 13, for use in the treatment, prognosis, diagnosis and/or monitoring of a neurological, neurovascular or neurodegenerative disorder, or as a neuroprotectant; optionally wherein the neurological, neurovascular or neurodegenerative disorder is age-related macular degeneration (AMD), Alzheimer's disease, amyotrophic lateral sclerosis, aneurysm, anti-MOG spectrum disorder, anti-aquaporin 4 spectrum disorder, brain tumor, brain oedema, CNS complication resulting from parasitic, bacterial, fungal or viral infection, demyelinating attack, encephalitis, intracranial lesion, intravertebral lesion, ischemic disorder, intracerebral hemorrhage, intracerebral bleeding TIA, epilepsy, temporal lobe epilepsy, Huntington's disease, multiple sclerosis, neuromyelitis optica spectrum disorder, myelin oligodendrocyte glycoprotein antibody-associated disease, stroke such as ischemic stroke and hemorrhagic stroke, thrombotic disorder, Parkinson's disease, multiple system atrophy, meningitis, traumatic brain injury, spinal cord injury, or damage of the optic nerve after acute vascular occlusion or in glaucoma.

What is claimed is:

1. A humanized anti-GluN1 antibody or an antigen-binding fragment or derivative thereof, wherein said antibody, antigen-binding fragment or derivative is specific for the glutamate ionotropic receptor NMDA type subunit 1 (GluN1) of the N-methyl-D-aspartate receptor (NMDAR), wherein said antibody, antigen-binding fragment or derivative inhibits interaction between the NMDAR and tissue-type plasminogen activator (t-PA) and/or inhibits NMDAR activation by t-PA;
wherein said antibody, fragment or derivative is capable of specifically binding to an epitope represented by the amino acid sequence EGRAAQKRLETLLEE (SEQ ID NO: 182) or AQKRL (SEQ ID NO: 183); or
wherein said antibody, fragment or derivative competes for binding with an antibody produced by a deposited hybridoma which is 15A4-B2-E5, 15A4-B2-F3, 15A4-B2, 6C9-A3, 6C9-A3-F4, or 6C9-A3-F6.

2. The humanized anti-GluN1 antibody, or antigen-binding fragment of claim 1, wherein said antibody, or antigen-binding fragment thereof, comprises:
(i) a heavy chain framework region comprising: a heavy chain FR1 comprising or consisting of the amino acid sequence shown in SEQ ID NO: 163, a heavy chain FR2 comprising or consisting of the amino acid sequence shown in SEQ ID NO: 164, a heavy chain FR3 comprising or consisting of the amino acid sequence shown in SEQ ID NO: 165, a heavy chain FR4 comprising or consisting of the amino acid sequence shown in SEQ ID NO: 166; or one or more heavy chain FR(s) having an amino acid sequence selected from the group consisting of SEQ ID NOs: 163-166 which is substituted at one or two amino acid positions or which contains a substitution of one or more amino acids or which is at least 80% identical to the amino acid sequence set forth in any one of SEQ ID NOs: 163-166, and which is functionally equivalent to the corresponding unaltered heavy chain FR(s);
and
(ii) a light chain framework region comprising: a light chain FR1 comprising or consisting of the amino acid sequence shown in SEQ ID NO: 144, a light chain FR2 comprising or consisting of the amino acid sequence shown in SEQ ID NO: 145, a light chain FR3 comprising or consisting of the amino acid sequence shown in SEQ ID NO: 146, a light chain FR4 comprising or consisting of the amino acid sequence shown in SEQ ID NO: 147; or one or more light chain FR(s) having an amino acid sequence selected from the group consisting of SEQ ID NOs: 144-147 which is substituted at one or two amino acid positions or which contains a substitution of one or more amino acids or which is at least 80% identical to the amino acid sequence set forth in any one of SEQ ID NOs: 144-147, and which is functionally equivalent to the corresponding unaltered light chain FR(s).

3. The humanized anti-GluN1 antibody, or antigen-binding fragment of claim 1, wherein said antibody, or antigen-binding fragment thereof, comprises:
(a) a heavy chain variable region comprising: a CDR-H1 comprising or consisting of the amino acid sequence shown in SEQ ID NO: 79 or 82, a CDR-H2 comprising or consisting of the amino acid sequence shown in SEQ ID NO: 80, 92, 94 or 95, a CDR-H3 comprising or consisting of the amino acid sequence shown in SEQ ID NO: 81; or
one or more CDR(s) having an amino acid sequence selected from the group consisting of SEQ ID NOs: 79-82, 92, 94 and 95 which is substituted at one or two amino acid positions or which contains a substitution of one or more amino acids, and which is functionally equivalent to the corresponding unaltered CDR(s);
(b) a light chain variable region comprising: a CDR-L1 comprising or consisting of the amino acid sequence shown in SEQ ID NO: 101, 128 or 132, a CDR-L2 comprising or consisting of the amino acid sequence shown in SEQ ID NO: 102 or 105, and a CDR-L3 comprising or consisting of the amino acid sequence shown in SEQ ID NO: 103, 106 or 117; or
one or more CDR(s) having an amino acid sequence selected from the group consisting of SEQ ID NOs: 101-103, 105, 106, 117, 128, and 132 which is substituted at one or two amino acid positions or which contains a substitution of one or more amino acids, and which is functionally equivalent to the corresponding unaltered CDR(s);
(c) a heavy chain framework region comprising: a heavy chain FR1 comprising or consisting of the amino acid sequence shown in SEQ ID NO: 163, a heavy chain FR2 comprising or consisting of the amino acid sequence shown in SEQ ID NO: 164, a heavy chain FR3 comprising or consisting of the amino acid sequence shown in SEQ ID NO: 165, a heavy chain FR4 comprising or consisting of the amino acid sequence shown in SEQ ID NO: 166; or
one or more heavy chain FR(s) having an amino acid sequence selected from the group consisting of SEQ ID NOs: 163-166 which is substituted at one or two amino acid positions or which contains a substitution of one or more amino acids or which is at least 80% identical to the amino acid sequence set forth in any one of SEQ ID NOs: 163-166, and which is functionally equivalent to the corresponding unaltered heavy chain FR(s);
and
(d) a light chain framework region comprising: a light chain FR1 comprising or consisting of the amino acid sequence shown in SEQ ID NO: 144, a light chain FR2 comprising or consisting of the amino acid sequence shown in SEQ ID NO: 145, a light chain FR3 comprising or consisting of the amino acid sequence shown in SEQ ID NO: 146, a light chain FR4 comprising or consisting of the amino acid sequence shown in SEQ ID NO: 147; or
one or more light chain FR(s) having an amino acid sequence selected from the group consisting of SEQ ID NOs: 144-147 which is substituted at one or two amino acid positions or which contains a substitution of one or more amino acids or which is at least 80% identical to the amino acid sequence set forth in any one of SEQ ID NOs: 144-147, and which is functionally equivalent to the corresponding unaltered light chain FR(s).

4. The humanized antibody, or antigen-binding fragment thereof, of claim 1, wherein said antibody, or antigen-binding fragment thereof, comprises:
(a) CDR-H1 comprising or consisting of the amino acid sequence shown in SEQ ID NO: 79, CDR-H2 comprising or consisting of the amino acid sequence shown in SEQ ID NO: 80, CDR-H3 comprising or consisting of the amino acid sequence shown in SEQ ID NO: 81, CDR-L1 comprising or consisting of the amino acid sequence shown in SEQ ID NO: 101, CDR-L2 comprising or consisting of the amino acid sequence shown in SEQ ID NO: 102, and CDR-L3 comprising or consisting of the amino acid sequence shown in SEQ ID NO: 103;
(b) CDR-H1 comprising or consisting of the amino acid sequence shown in SEQ ID NO: 82, CDR-H2 comprising or consisting of the amino acid sequence shown in SEQ ID NO: 80, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106;
(c) CDR-H1 comprising or consisting of the amino acid sequence shown in SEQ ID NO: 82, CDR-H2 comprising or consisting of the amino acid sequence shown in SEQ ID NO: 92, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106;
(d) CDR-H1 comprising or consisting of the amino acid sequence shown in SEQ ID NO: 82, CDR-H2 comprising or consisting of the amino acid sequence shown in SEQ ID NO: 94, CDR-H3 comprising or consisting of the amino acid sequence shown in SEQ ID NO: 81, CDR-L1 comprising or consisting of the amino acid sequence shown in SEQ ID NO: 128, CDR-L2 comprising or consisting of the amino acid sequence shown in SEQ ID NO: 105, and CDR-L3 comprising or consisting of the amino acid sequence shown in SEQ ID NO: 106;
(e) CDR-H1 comprising or consisting of the amino acid sequence shown in SEQ ID NO: 82, CDR-H2 comprising or consisting of the amino acid sequence shown in SEQ ID NO: 94, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 117; or
(f) CDR-H1 comprising or consisting of the amino acid sequence shown in SEQ ID NO: 82, CDR-H2 comprising or consisting of the amino acid sequence shown in SEQ ID NO: 95, CDR-H3 comprising or consisting of the amino acid sequence shown in SEQ ID NO: 81, CDR-L1 comprising or consisting of the amino acid sequence shown in SEQ ID NO: 132, CDR-L2 comprising or consisting of the amino acid sequence shown in SEQ ID NO: 105, and CDR-L3 comprising or consisting of the amino acid sequence shown in SEQ ID NO: 106.

5. The humanized antibody, or antigen-binding fragment thereof, of claim 1 wherein said antibody, or antigen-binding fragment comprises or consists of any one of heavy chain variable regions (VH) selected from the group consisting of: SEQ ID NO: 7, 8, 9, 10, 11, 12, 13, 14, 15, 16, 17, 18, 19, 20, 21, 22, 25, 26, 27, 28, 29, 30, 31, 32, 33; and/or wherein said antibody, or antigen-binding fragment comprises or consists of any one of light chain variable regions (VL) selected from the group consisting of: SEQ ID NO: 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 71, 72, 73, 74, 75, 76, 77, 78; or a variant sequence thereof which differs by only one, two or three amino acids, or which has at least or about 85% sequence identity thereto.

6. The humanized antibody, or antigen-binding fragment thereof, of claim 1, wherein said antibody, or antigen-binding fragment is capable of binding to at least a portion of a region of an NMDA receptor that interacts with tissue-type plasminogen activator (t-PA) and wherein said binding inhibits the interaction between the NMDA receptor and t-PA, such that at least one function of the NMDA receptor activated by t-PA is selectively inhibited.

7. The humanized antibody, or antigen-binding fragment thereof, of claim 1, wherein said antibody, antigen-binding fragment is a monoclonal antibody, or an antigen-binding fragment thereof.

8. The humanized antibody, or antigen-binding fragment thereof, of claim 5, wherein said antibody, or antigen-binding fragment, comprises:
(i) a heavy chain variable region comprising the amino acid sequence of any one of SEQ ID NOs: 7-22 or 25-33; and/or
(ii) a light chain variable region comprising the amino acid sequence of any one of SEQ ID NOs: 34-52 or 54-78.

9. The humanized antibody, fragment or derivative of claim 1, wherein said antibody, or antigen-binding fragment thereof, comprises:
a heavy chain variable region comprising or consisting of the amino acid sequence of SEQ ID NO: 7, and a light chain variable region comprising or consisting of the amino acid sequence of SEQ ID NO: 34;
a heavy chain variable region comprising or consisting of the amino acid sequence of SEQ ID NO: 12, and a light chain variable region comprising or consisting of the amino acid sequence of SEQ ID NO: 65;
a heavy chain variable region comprising or consisting of the amino acid sequence of SEQ ID NO: 14, and a light chain variable region comprising or consisting of the amino acid sequence of SEQ ID NO: 65;
a heavy chain variable region comprising or consisting of the amino acid sequence of SEQ ID NO: 17, and a light chain variable region comprising or consisting of the amino acid sequence of SEQ ID NO: 74;
a heavy chain variable region comprising or consisting of the amino acid sequence of SEQ ID NO: 17, and a light chain variable region comprising or consisting of the amino acid sequence of SEQ ID NO: 75; or
a heavy chain variable region comprising or consisting of the amino acid sequence of SEQ ID NO: 32, and a light chain variable region comprising or consisting of the amino acid sequence of SEQ ID NO: 65.

10. The antibody, or antigen-binding fragment thereof, of claim 1, wherein said antibody, or antigen-binding fragment thereof, comprises:
(i) a heavy chain comprising or consisting of the amino acid sequence of SEQ ID NO: 14; and
(ii) a light chain comprising or consisting of the amino acid sequence of SEQ ID NO: 65

11. The antibody, or antigen-binding fragment thereof, of claim 1, wherein said antibody, or antigen-binding fragment thereof, comprises:
(i) a heavy chain comprising or consisting of the amino acid sequence of SEQ ID NO: 7; and/or
(ii) a light chain comprising or consisting of the amino acid sequence of SEQ ID NO: 34.

12. A nucleic acid encoding the antibody, antigen-binding fragment or derivative of claim 1.

13. An expression cassette or vector comprising the nucleic acid according to claim 12, optionally comprising a promoter operatively connected with said nucleic acid.

14. A host cell comprising the nucleic acid of claim 12.

15. A pharmaceutical composition comprising the antibody, antigen-binding fragment or derivative of claim 1, and a pharmaceutically acceptable carrier, diluent or excipient, optionally wherein the pharmaceutical composition is suitable for administration subcutaneously, intramuscularly, intravenously, intraperitoneally, or orally.

16.-18. (canceled)

19. A host cell comprising the expression cassette or vector of claim 13.

20. A method for the treatment or prevention of a neurological, neurovascular or neurodegenerative disorder in a subject, or for providing neuroprotection to the subject, comprising administering the antibody, antigen-binding fragment or derivative of claim 1 to the subject, such that the neurological, neurovascular or neurodegenerative disorder is treated or prevented in the subject or neuroprotection is provided to the subject.

21. The method of claim XX, wherein the neurological, neurovascular or neurodegenerative disorder is age-related macular degeneration (AMD), Alzheimer's disease, amyotrophic lateral sclerosis, aneurysm, anti-MOG spectrum disorder, anti-aquaporin 4 spectrum disorder, brain tumor, brain oedema, CNS complication resulting from parasitic, bacterial, fungal or viral infection, demyelinating attack, encephalitis, intracranial lesion, intravertebral lesion, ischemic disorder, intracerebral hemorrhage, intracerebral bleeding TIA, epilepsy, temporal lobe epilepsy, Huntington's disease, multiple sclerosis, neuromyelitis optica spectrum disorder, myelin oligodendrocyte glycoprotein antibody-associated disease, stroke such as ischemic stroke and hemorrhagic stroke, thrombotic disorder, Parkinson's disease, multiple system atrophy, meningitis, traumatic brain injury, spinal cord injury, or damage of the optic nerve after acute vascular occlusion or in glaucoma.

22. A method of in vivo diagnosis of a neurological, neurovascular or neurodegenerative disorder in a subject comprising administering the antibody, antigen-binding fragment or derivative of claim 1 to the subject.
